SEQUENCE LISTING (34 sequences)

SEQ ID NO: 1 - protein, 238 aa, synthetic construct
MSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 2 - protein, 237 aa, synthetic construct
MRGSHHHHHHGSAHGLTDDMTMHFRMEGCVDGHKFVIEGNGNGNPFKGKQFINLCVIEGGPLPFSEDILSAAFXNRLFTEYPEGIVDYFKNSCPAGYTWHRSFRFEDGAVCICSADITVNVRENCIYHESTFYGVNFPADGPVMKKMTTNWEPSCEKIIPINSQKILKGDVSMYLLLKDGGRYRCQFDTIYKAKTEPKEMPDWHFIQHKLNREDRSDAKNQKWQLIEHAIASRSALP

SEQ ID NO: 3 - protein, 225 aa, synthetic construct
KGEELFTGVVPILVELDGDVNGHKFSVSGEGEGDATYGKLTLKFICTTGKLPVPWPTLVTTFXLQCFARYPDHMKRHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGI

SEQ ID NO: 4 - protein, 236 aa, synthetic construct
MSKGEELFTGVVPILVELDGDVNGHKFSVSGEGEGDATYGKLTLKFICTTGKLPVPWPTLVTTFXVQCFSRYPDHMKRHDFFKSAMPEGYVQERTIFFKDDGNYKTRAEVKFEGDTLVNRIELKGIDFKEDGNILGHKLEYNFNSHNVYIMADKQKNGIKVNFKIRHNIEDGSVQLADHYQQNTPIGDGPVLLPDNHYLSTQSALSKDPNEKRDHMVLLEFVTAAGITHGMDELYK

SEQ ID NO: 5 - protein, 237 aa, synthetic construct
MVSKGEELFTGVVPILVELDGDVNGHKFSVSGEGEGDATYGKLTLKFICTTGKLPVPWPTLVTTLXVQCFARYPDHMKQHDFFKSAMPEGYVQERTIFFKDDGNYKTRAEVKFEGDTLVNRIELKGIDFKEDGNILGHKLEYNAISDNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 6 - protein, 237 aa, synthetic construct
MVSKGEELFTGVVPILVELDGDVNGHKFSVSGEGEGDATYGKLTLKFICTTGKLPVPWPTLVTTLXVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 7 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 8 - protein, 311 aa, synthetic construct
MTSKVYDPELRKRMITGPQWWARCKQMNVLDSFINYYDSEKHAENAVIFLHGNAASSYLWRHVVPHVEPVARCIIPDLIGMGKSGKSGNGSYRLLDHYKYLTEWFKHLNLPKKIIFVGHDWGACLAFHYCYEHQDRIKAVVHAESVVDVIESWDEWPDIEEDIALIKSEEGEKMVLENNFFVETMLPSKIMRKLEPEEFAAYLEPFKEKGEVRRPTLSWPREIPLVKGGKPDVVEIVRNYNAYLRASHDLPKMFIESDPGFFSNAIVEGAKKFPNTEFVKVKGLHFSQEDAPDEMGNYIKSFVERVLKNEQ

SEQ ID NO: 9 - protein, 550 aa, synthetic construct
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGSPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNDPEATNALIDKDGWLHSGDIAYWDEDEHFFIVDRLKSLIKYKGCQVAPAELESILLQHPNIFDAGVAGLPGDDAGELPAAVVVLEHGKTMTEKEIVDYVASQVTTAKKLRGGVVFVDEVPKGLTGKLDARKIREILIKAKKGGKSKL

SEQ ID NO: 10 - protein, 805 aa, Homo sapiens [sequence omitted]

SEQ ID NO: 11 - protein, 550 aa, Homo sapiens; identical to SEQ ID NO: 9

SEQ ID NO: 12 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 13 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 14 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 15 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 16 - protein, 492 aa, Homo sapiens
MALNSGSPPAIGPYYENHGYQPENPYPAQPTVVPTVYEVHPAQYYPSPVPQYAPRVLTQASNPVVCTQPKSPSGTVCTSKTKKALCITLTLGTFLVGAALAAGLLWKFMGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWIVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 17 - protein, 25 aa, synthetic construct
MSKRGASNEMALLLLLLGLLQQAMA

SEQ ID NO: 18 - DNA, 75 bp, synthetic construct
  source 1..75: mol_type = other DNA
atgagcaagaggggcgccagcaacgagatggccctgctgctgctgctgctgggcctgctgcagcaggccatggcg

SEQ ID NO: 19 - protein, 18 aa, synthetic construct
MKWVTFISLLFLFSSAMA

SEQ ID NO: 20 - DNA, 54 bp, synthetic construct
  source 1..54: mol_type = other DNA
atgaagtgggtgaccttcatcagcctgctgttcctgttcagcagcgccatggcg

SEQ ID NO: 21 - protein, 25 aa, synthetic construct
MSKRGASNEMALLLLLLGLLASVLA

SEQ ID NO: 22 - DNA, 74 bp, synthetic construct
  source 1..74: mol_type = other DNA
atgagcaagaggggcgccagcaacgagatggccctgctgctgctgctgctgggcctgctggctacgttttagca

SEQ ID NO: 23 - protein, 18 aa, synthetic construct
MKWVTFISLLFLFASVLA

SEQ ID NO: 24 - DNA, 53 bp, synthetic construct
  source 1..53: mol_type = other DNA
atgaagtgggtgaccttcatctccctgcttgcctgttcgctagcgttttagca

SEQ ID NO: 25 - DNA, 20 bp, synthetic construct
  source 1..20: mol_type = other DNA
tcctgagtccggaccacttt

SEQ ID NO: 26 - DNA, 22 bp, synthetic construct
  source 1..22: mol_type = other DNA
caccgcatgttagaagacttcc

SEQ ID NO: 27 - DNA, 23 bp, synthetic construct
  source 1..23: mol_type = other DNA
acctccagttaggaaaggggact

SEQ ID NO: 28 - DNA, 25 bp, synthetic construct
  source 1..25: mol_type = other DNA
aagtttttcttgaaaacccatggaa

SEQ ID NO: 29 - DNA, 21 bp, synthetic construct
  source 1..21: mol_type = other DNA
ccttgtagatgaactcgccgt

SEQ ID NO: 30 - DNA, 22 bp, synthetic construct
  source 1..22: mol_type = other DNA
ggtgtcgtgattattcgaaggg

SEQ ID NO: 31 - DNA, 21 bp, synthetic construct
  source 1..21: mol_type = other DNA
ggtgggaggtctatataagca

SEQ ID NO: 32 - DNA, 18 bp, synthetic construct
  source 1..18: mol_type = other DNA
ccagaggtgctcttggag

SEQ ID NO: 33 - protein, 22 aa, synthetic construct
MKYLLPTAAAGLLLLAAQPAMA

SEQ ID NO: 34 - DNA, 66 bp, synthetic construct
  source 1..66: mol_type = other DNA
atgaagtatttgttgccgacggcggcggcggggttgttgttgttggcggcgcagccggccatggcg

(19) United States
(12) Patent Application Publication
(10) Pub. No.: US 2023/0221313 A1
(43) Pub. Date: Jul. 13, 2023

(54) ANALYSIS, SCREENING, AND SELECTION FOR SOLUBLE PROTEIN FUNCTION IN SECRETED PROTEIN CELL LIBRARIES

(71) Applicants: Massachusetts Institute of Technology, Cambridge, MA (US); The General Hospital Corporation, Boston, MA (US); University of Kansas, Lawrence, KS (US)

(72) Inventors: Brandon DeKosky, Cambridge, MA (US); Matias F. Gutierrez, Cambridge, MA (US); Chengyu Chung, Lawrence, KS (US); Ahmed Fahad, Lawrence, KS (US); Nicoleen Boyle, Lawrence, KS (US); Shuyan Jin, Lawrence, KS (US)

(21) Appl. No.: 18/054,823

(22) Filed: Nov. 11, 2022

Related U.S. Application Data
(60) Provisional application No. 63/398,085, filed on Aug. 15, 2022.
(60) Provisional application No. 63/299,315, filed on Jan. 13, 2022.

(51) Int. Cl. G01N 33/566; G01N 33/569

(52) U.S. Cl. CPC G01N 33/566; G01N 33/56983; G01N 2500/02

(57) ABSTRACT

Disclosed herein are methods, compositions, systems, and kits related to functional testing of soluble polypeptides in a single-cell format.

### CROSS-REFERENCE TO RELATED APPLICATIONS

This application claims priority to U.S. Provisional Application 63/299,315 filed on Jan. 13, 2022, and U.S. Provisional Application 63/398,085 filed on Aug. 15, 2022. The entire content of both applications is incorporated herein by reference.

### STATEMENT REGARDING FEDERALLY SPONSORED RESEARCH OR DEVELOPMENT

This invention was made with government support under DP50D23118 awarded by the United States National Institutes of Health. The government has certain rights in the invention.

### REFERENCE TO AN ELECTRONIC SEQUENCE LISTING

The contents of the electronic sequence listing (631020.00153.xml; Size: 36,358 bytes; and Date of Creation: Nov. 8, 2022) is herein incorporated by reference in its entirety.

### FIELD

The present technology relates generally to methods and compositions useful for the analysis and screening of soluble peptides, for example, as applied to the field of drug discovery. The methods, systems, kits, and compositions disclosed herein provide tools for rapidly, efficiently, and accurately screening and selecting active antibodies, proteins, or peptides from large libraries of antibodies, proteins, or peptides.

### BACKGROUND

Many assays for drug discovery that analyze soluble protein function require substantial quantities of purified proteins and use low- or medium-throughput (<10,000) assays to test protein function in well plates. Examples include cell-based assays, viral neutralization assays, or cellular activity-based protein functional activation assays. Most importantly for biotechnology discovery purposes, the process of expressing, purifying, and analyzing protein is not readily compatible with direct selection of functional protein or peptide variants from variant libraries. Important examples of drug classes that often require soluble screening or cell activity-based assays to test for function include antibody, protein, or peptides that neutralize viruses, antibody, protein, or peptides that activate surface cellular receptors, and antibody, protein, or peptides that block the activation of surface cellular receptors. Therefore, a need exists for improved and rapid assays for soluble protein or peptide function.

### SUMMARY

Disclosed herein are methods, compositions, systems, and kits related to functional testing of soluble polypeptides in a single-cell format.

In some aspects, a screening method is provided.

In some embodiments, the methods comprises: (a) detecting the presence and/or level of expression of a reporter molecule in a single, isolated, genetically engineered cell, wherein the cell presents a cell surface protein; and wherein the cell is engineered to: (i) secrete a heterologous test polypeptide; and (ii) express a reporter molecule if the test polypeptide activates the cell surface protein.

In some embodiments, the method comprises: (a) detecting the presence and/or level of expression of a reporter molecule in a single, isolated, genetically engineered cell, wherein the cell presents a cell surface protein; and wherein the cell is engineered to: (i) secrete a heterologous test polypeptide; and (ii) express a reporter molecule if the test polypeptide does not activate the cell surface protein.

In some embodiments, the method comprises: (a) contacting a single, isolated, genetically engineered cell with a test reagent, wherein the cell presents a cell surface protein, and wherein the cell is engineered to: (i) secrete a heterologous test polypeptide; and; (ii) express a reporter molecule if one of the test polypeptide or the test reagent activates the cell surface protein; (b) detecting the presence and/or level of expression of the reporter molecule.

In some embodiments, the method comprises: (a) contacting a single, isolated, genetically engineered cell with a test reagent comprising a reporter molecule, wherein the cell presents a cell surface protein; wherein the test reagent is capable of binding the cell surface protein presented by the cell, forming a reagent-receptor complex, and wherein the test reagent gains entry into the cell when the reagent-receptor complex is formed; wherein the cell is engineered to: (i) secrete a heterologous test polypeptide; (b) detecting the presence and/or level of expression of the reporter molecule in the cells.

In some embodiments of the previously described methods, the cell comprises a mammalian cell, an insect cell, an avian cell, a yeast cell, a fungal cell, a plant cell, or a bacterial cell.

In some embodiments, the cell surface protein comprises an endogenous protein. In some embodiments, the cell is engineered to express a cell surface protein. In some embodiments, the cell surface protein comprises a heterologous protein.

In some embodiments, secretion of the test polypeptide is constitutive. In some embodiments, the secretion of the test polypeptide is inducible.

In some embodiments, the single, isolated, genetically engineered cell is in a well of a multi-well plate, in a chamber of a microchip in a microfluid droplet, such as an emulsion droplet, or in a Nanopen™.

In some embodiments of the previously described methods, the reporter molecule comprises a fluorescent marker, an enzyme, a tagged protein, or a nucleic acid sequence.

In some embodiments, the cell comprises a human cell.

In some embodiments, the reporter molecule comprises a nucleic acid sequence, optionally a barcode sequence, and detecting the presence and/or level of expression of the reporter molecule comprises one or more of an amplification reaction and a sequencing reaction, optionally a single cell sequencing reaction.

In some embodiments, the reporter molecule comprises a fluorescent moiety, and detecting the presence and/or level of expression of the reporter molecule comprises fluorescence activated cell sorting.

In some embodiments, the method further comprises sequencing the nucleic acids encoding the heterologous test polypeptide.

In some embodiments, the heterologous test peptide comprises a variant of the receptor ligand.

In some embodiments, the variant is derived from a library of ligand variants.

In some embodiments, the test polypeptide comprises a variant of a cell surface protein ligand, and the test reagent comprises an agonist or an antagonist of protein activation by the wild-type ligand.

In some embodiments, the test reagent comprises a cell surface protein ligand, and the test polypeptide is derived from a library of potential agonists or antagonists of receptor activation by the ligand. In some embodiments, the test polypeptide comprises an antibody or antigen binding fragment thereof. In some embodiments, the antibody or antigen binding fragment is derived from a library of antibodies, or antigen binding fragments.

In some embodiments, the test reagent comprises one or more of a virus, virus-like particle, pseudoviruses, and recombinant viral particle, and wherein the cell surface protein comprises a component of viral entry into the cell. In some embodiments, the virus is selected from Coronavirus A, B, C, or D, Flavivirus, Lentivirus, Influenza A, B, or C. In some embodiments, the virus selected from HIV, SARS-CoV-2, Epstein-Barr virus, herpes simplex virus, cytomegalovirus, respiratory syncytial virus, Ebola virus, Marburg virus, Dengue virus, and Yellow Fever Virus. In some embodiments, the virus comprises a SARS-CoV-2 virus, and wherein the cell surface protein comprises a human angiotensin-converting enzyme 2 (hACE2), and in some embodiment, the cell is engineered to express Transmembrane Serine Protease 2 (TMPRSS2).

In some aspects, a composition, kit, or system, comprising the genetically engineered cell of any of the previous embodiments is provided.

In some aspects, a kit is provided.

In some embodiments, the kit comprises: (a) a vector encoding a heterologous test polypeptide; (b) a vector encoding a reporter molecule, expression of which is activated if the heterologous test polypeptide activates a cell surface protein, optionally, wherein one or more of the vectors are expression vectors, or, optionally, wherein one or more of the vectors are integration vectors.

In some embodiments, the kit comprise (a) a vector encoding a heterologous test polypeptide; (b) a vector encoding a reporter molecule, expression of which is activated if the heterologous test polypeptide does not activate a cell surface protein, optionally, wherein one or more of the vectors are expression vectors, or, optionally, wherein one or more of the vectors are integration vectors.

In some embodiments, a kit comprises: (1) a test reagent and (2) (a) a vector encoding a heterologous test polypeptide; (b) a vector encoding a reporter molecule, expression of which is activated if either the heterologous test polypeptide or test reagent activates a cell surface protein, optionally, wherein one or more of the vectors are expression vectors, or, optionally, wherein one or more of the vectors are integration vectors.

In some embodiments, a kit comprises: (1) a test reagent comprising a reporter molecule and (2) (a) a vector encoding a heterologous test polypeptide, optionally, wherein one or more of the vectors are expression vectors, or, optionally, wherein one or more of the vectors are integration vectors.

In some embodiments of any of the aforementioned kits, one or more nucleic acids further encode (c) a cell surface protein. In some embodiments, the heterologous test polypeptide is operably linked to a promoter. In some embodiments, the promoter is a constitutive promoter. In some embodiments, the promoter is an inducible promoter.

In some embodiments, the reporter molecule comprises a fluorescent marker, an enzyme, a tagged protein, or a nucleic acid sequence.

In some embodiments, the test reagent comprises a virus, virus-like particle, pseudoviruses, and recombinant viral particle. In some embodiments, the virus is selected from a Coronavirus A, B, C, or D, Flavivirus, Lentivirus, and Influenza A, B, or C. In some embodiments, the virus is selected from HIV, SARS-CoV-2, Epstein-Barr virus, herpes simplex virus, cytomegalovirus, respiratory syncytial virus, Ebola virus, Marburg virus, Dengue virus, and Yellow Fever Virus. In some embodiments, the pseudovirus comprises a peptide, polypeptide, or protein derived from a Coronavirus A, B, C, or D, Flavivirus, Lentivirus, or Influenza A, B, or C. In some embodiments, the pseudovirus comprises a peptide, polypeptide, or protein derived from HIV, SARS-CoV-2, Epstein-Barr virus, herpes simplex virus, cytomegalovirus, respiratory syncytial virus, Ebola virus, Marburg virus, Dengue virus, or Yellow Fever Virus.

In some embodiments, the heterologous test peptide comprises an antibody, or a portion thereof. In some embodiments, the heterologous test peptide is a single chain variable fragment (scFv) or a nanobody.

In some embodiments, a kit comprises: (1) a vector for expressing a heterologous test polypeptide, (2) a genetically engineered cell comprising: (a) a nucleic acid encoding a reporter, expression of which is activated if the heterologous test polypeptide activates a cell surface protein.

In some embodiments, a kit comprises: (1) a vector for expressing a heterologous test polypeptide, (2) a genetically engineered cell comprising: (a) a nucleic acid encoding a reporter, expression of which is activated if the heterologous test polypeptide does not activate a cell surface protein.

In some embodiments, a kit comprises: (1) a vector for expressing a heterologous test polypeptide, (2) a genetically engineered cell comprising: (a) a nucleic acid encoding a reporter, expression of which is activated if the heterologous test polypeptide activates a cell surface protein, and optionally, (3) a test reagent.

In some embodiments, a kit comprises: (1) a vector for expressing a heterologous test polypeptide, (2) a genetically engineered cell; (3) a test reagent comprising a reporter molecule, wherein the test reagent is capable of binding a cell surface protein presented by the cell, forming a reagent-receptor complex, and wherein the test reagent gains entry into the cell when the reagent-receptor complex is formed.

In some embodiments of any of the aforementioned kits, the genetically engineered cell further comprises: (c) a nucleic acid encoding a heterologous cell surface protein.

### BRIEF DESCRIPTION OF THE DRAWINGS

FIG. 1a-b. (a) SARS-CoV-2 and (b) HIV receptors expression plasmids used to modify cell lines to make them permissible to virus or pseudovirus entry.

FIG. 2. The generation of cells expressing ACE2 and TMPRSS2 for SARS-CoV-2 infection, which are also capable of antibody secretion, to enable large-scale compartment-based library screening for antibody SARS-CoV-2.

FIG. 3. Generating a cell line include ACE2, TMPRSS2 and IgG gene allowing neutralization assay to be performed in a single cell basis by linking protein secretion (in this case, an IgG) to viral infection along with a functional readout for infection.

FIG. 4a-b. Cell line development for single-cell SARS-CoV-2 neutralization assays. (a) Incorporation of ACE2 and TMPRSS2 into the site-specific TARGATT cell line for transgene insertion. (b) Paired VH:VL library cloning in TARGATT cells.

FIG. 5. Evaluation of SARS-CoV-2 Pseudovirus infectivity using different amount of virus through flow cytometry analysis.

FIG. 6a-b. Vector maps of (a) pCMV-EF1a vector and (b) pBI vector, two examples of vectors that can enable protein or peptide secretion. In this case, the secreted protein is an IgG.

FIG. 7. ELISA quantification comparison of IgG yield for transient IgG expression. Different leader peptide sequence combinations can provide different levels of secreted protein expression.

FIG. 8. The FRT/FLP based site-directed integration system for IgG expression.

FIG. 9. Integrase-based site-directed integration system for IgG expression.

FIG. 10. CRISPR/Cas9 homologous-directed repair system for IgG expression into cell lines for analysis of soluble protein function.

FIG. 11. Overview of several possible secreted protein expression platforms for library cloning into mammalian cells for secreted protein assays.

FIG. 12a-b. Expression of IgG in a single-directional format. (a) Expression of IgG as single-chain variable fragment. (b) Expression of full IgG in a bi-cistronic format with a p2A cleavage peptide.

FIG. 13. Neutralizing activity of VRC01 and 910-30 via flow cytometry of HEKACE2 cells.

FIG. 14a-b. Linked antibody secretion and SARS-CoV-2 infection for neutralization assays. (a) ELISA standard curve for IgG secreted by HEK293-ACE2. (b) 96-well neutralization assays for HEK293-ACE2 cells expressing neutralizing mAbs (first, second, fifth, and sixth group) or non-neutralizing mAbs (third and fourth group), with two different leader peptides (LP4 or LP5). Secreted mAb concentration is reported above each bar. IgG-secreting cells prevented pseudovirus infection. 910-30 SARS-CoV-2 IC₅₀ is approximately 0.2 μg/mL.

FIG. 15a-b. Single-cell isolation and antibody secretion inside emulsion droplets. (a) Single cells were encapsulated in 80 μm droplets and analyzed by light microscopy. (b) Cells were incubated and secreted antibody, either in bulk cell culture or inside droplets. Supernatants were recovered and analyzed by ELISA to determine antibody concentrations (avg. +/−st. dev.). The concentration in droplets quickly exceeded 0.5 μg/mL by Day 2. *Extrapolation slightly above the standard curve.

FIG. 16a-c. Example of high-throughput single-cell neutralization assay for mapping natively paired human antibodies against diverse SARS-CoV-2 variants. (a) Single TARGATT-HEK293-ACE2 cells secreting antibodies are captured inside emulsion droplets. (b) After around 24 h of antibody secretion, single cell droplets are merged with SARS-CoV-2 pseudovirus droplets. Cells secreting neutralizing antibody at sufficient concentration are protected from infection. (c) Cells are sorted into GFP− and GFP+ populations. Non-infected GFP− cells can be passaged for multiple screening rounds. DNA amplicons of sorted libraries are recovered for quantitative analysis and subsequent antibody expression. The renewable libraries can be screened repeatedly against diverse SARS-CoV-2 pseudoviruses separately, or against pseudovirus panels, to select for broad vs. strain-specific antibodies.

FIG. 17. Yellow fever virus (YFV) neutralization detection in cells secretion anti-YFV monoclonal antibodies. Cells secreting mAb-17 were protected from YFV RVP infection; cells not expressing mAb-17 were infected by RVPs as demonstrated by expressed GFP after RVP exposure.

FIG. 18a-d. ELISA quantification of antibody expression using different leader peptide and promoter combinations. (a) Table of Leader Peptide Amino Acid Sequences and Leader Peptide Pair Names. (b) Plasmid illustration of minimal human cytomegalovirus (miniCMV) bi-directional promoter to drive expression of heavy and light chains of antibody and dual promoters consisting of CMV to express the heavy chain of the antibody and human elongation factor-1 alpha (Ef1α) driving expression of the light chain. (c) Sandwich ELISA quantification of VRC01 transient expression levels with different leader peptide combinations in each vector. (d) Sandwich ELISA quantification of CR3022 transient expression levels with different leader peptide combinations in each vector.

FIG. 19. A CRISPR-Cas9 integration system for antibody secretion in mammalian cells.

FIG. 20. Neutralization was demonstrated using CRISPR-Cas9 integration system for antibody secretion.

FIG. 21. Quantification of cell-secreted antibodies demonstrated the use of CRISPR-Cas9 to achieve antibody secretion.

FIG. 22. Verification of CRISPR-Cas9-based genomic insertion of antibody genes into mammalian cells.

FIG. 23. TARGATT gene integration of the mAb 2-15 sequence.

FIG. 24. Neutralization activity of an anti-SARS-CoV2 antibody, 2-15, secreted from the TARGATT12-15 cells.

FIG. 25. Quantification of antibody secretion from the TARGATT2-15 cells.

FIG. 26a-b. (a) Gel electrophoresis of the genomic PCR using a downstream primer set to validate the successful gene-integration of TARGATT2-15 cells. (b) A PCR reaction using a human control primer set as an internal PCR control (panel b).

FIG. 27. HIV-1 neutralization detection in cells secreting anti-HIV-1 monoclonal antibodies. Cells secreting VRC34 were protected from HIV-1 pseudovirus infection; cells not expressing VRC34 were infected by pseudovirus as demonstrated by expressed GFP after W6M.EnV.C2 HIV-1 pseudovirus exposure. n=2 replicates were performed at each condition. The initial cell density was 2,500 cells/well. Dilutions were made with pseudovirus particles; the number of cells and antibody concentrations were held constant across pseudovirus dilutions. WT-wild type, NC-negative control (no pseudovirus particles added).

FIG. 28. Droplet merging using electrocoalescence. Top: Droplet merger is off.

Droplets containing cells and droplets containing rhodamine are clearly separated, both in the bright field and when measuring rhodamine fluorescence. Bottom: Droplet merger is on using an electric field, with settings at 1.6 V. Droplet containing cells merge with rhodamine 110 dye for visibility using microscopy, as shown in rhodamine 110 channel. Arrows indicate the presence of cells inside droplets. No rhodamine is present in the cell-containing droplets when the droplet merger voltage is “off”, whereas rhodamine is present inside droplets containing cells when the droplet merger voltage is “on”, indicating successfully merged droplets.

FIG. 29. PCR amplification of variable heavy chain sequences from cell lines analyzed in high-throughput assays. Cell population libraries were sorted for GFP- or GFP+ expression prior to DNA recovery using a flow cytometer. These data demonstrate our ability to recover the DNA sequences of cells utilized in high-throughput droplet-based cell secretion protein functional assays.

FIG. 30. SARS-CoV-2 droplet neutralization assay implementation with synthetic libraries. HEK293/ACE2 cells expressing either VRC01, CR3022 910-30 or mAb 1-20 were pooled and single cells were captured and allowed to secrete antibody for 24 hours. Droplet-containing cells and antibody were merged with droplets containing SARS-CoV-2 D614G RVPs allowing infection for 24 hours. After infection, cells were recovered from the droplets and allowed. Two days later, GFP−/mCherry+ (not infected cells/mAb producing) and GFP+/mCherry+ (infected cells/mAb producing) cells were sorted. gDNA was extracted from both populations for sequencing, while 10% of the recovered GFP−/mCherry+ cells were expanded for a second round of droplet neutralization assay. Zero reads were observed in GFP+ populations for some clones, reflecting a total lack of infection events for those neutralizing antibody clones and providing the expected outcome with very high assay precision. Division calculations for clonal fraction of read fold-changes, defined as (GFP− read prevalence/GFP+ read prevalence), can result in a divide by zero error when zero reads are available (indicating complete neutralization inside droplets for certain antibody clones, for example). Mathematically the closest approximation for a divide by zero error would be infinity, however those fold changes were artificially estimated here at a value of 9,999 for the purposes of comparison to other clones.

FIG. 31. Droplet neutralization assay using HIV-1 pseudovirus with synthetic libraries. TZM/GFP cells expressing either 72A1, VRC01 or VRC34 were pooled. Next, single cells were captured and allowed to secrete antibody for 24 hours. Droplet-containing cells and antibody were next merged with droplets containing HIV pseudoviruses (generated using the sequence BG505.W6M.Env.C2) allowing infection for 24 hours. After infection, cells were recovered from the droplets and allowed. Two days later, GFP−/mCherry+ (not infected cells/mAb producing) and GFP+/mCherry+ (infected cells/mAb producing) cells were sorted. gDNA was extracted from both populations. The non-neutralizing antibody (72A1) was greatly enriched in the GFP+ population, indicating low neutralization activity. This figure demonstrates the ability to successfully implement neutralization assays inside droplets for HIV-1 pseudovirus assays using NGS analysis of sorted cell libraries.

### DETAILED DESCRIPTION

The present invention is described herein using several definitions, as set forth below and throughout the application.

Unless otherwise specified or indicated by context, the terms “a”, “an”, and “the” mean “one or more.” For example, “an inhibitor of tumor cell aggregation” should be interpreted to mean “one or more inhibitors of tumor cell aggregation.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “protein,” “peptide,” and “polypeptide” are used interchangeably.

As used herein, the term “microwell” is defined as an enclosed or partially enclosed compartment with its diameter or width in at least one dimension between 0.1 microns and 4,999 microns. Either one, two, or zero of the other dimensions of the microwell may be open and connected to a broader reservoir.

Disclosed herein are methods, compositions, systems, and kits for the functional screening of soluble protein libraries in a rapid, high throughput, and cost-effective manner.

By way of example and as described herein, a cell line was generated that was permissible to viral infection and concurrent antibody secretion to analyze the viral neutralization features of the produced antibodies.

In some embodiments, a cell line is produced that is susceptible to SARS-CoV-2 infection, and that also secretes antibodies, or antigen binding fragments thereof. In some embodiments, the ability of the secreted antibodies to neutralize, prevent, or reduce viral infection (SARS-coV-2 infection) of the antibody secreting cell is analyzed.

In some embodiments, a cell line is produced that is susceptible to HIV infection, and that also secretes anti-HIV antibodies. In some embodiments, the ability of the secreted antibodies to neutralize, prevent, or reduce viral infection (HIV infection) of the antibody secreting cell is analyzed.

In some embodiments, a cell line that is already permissible to viral infection (e.g., Raji-DC-SIGN with yellow fever virus recombinant viral particles) is used.

In some embodiments, antibody expression may be engineered into a mammalian cell line that is natively capable of virus infection. In some embodiments, the cell line is engineered to express at least one component of viral entry (e.g., a heterologous cell surface molecule).

In some embodiments, a heterologous polypeptide (such as a potential ligand or a potential ligand-receptor antagonist or agonist) is expressed in a cell line that has been generated for the purpose of analysis of ligand-receptor agonism or antagonism (e.g., for the PD-1 surface receptor).

Methods of Screening

Alternative approaches for functional analysis of secreted polypeptide molecules currently known in the prior art (e.g., as shown by the microfluidics functional sorting services sold by the AbCheck company, as well by the example by Lin et al., (Lin, W. et al., (2022). Rapid microfluidic platform for screening and enrichment of cells secreting virus neutralizing antibodies. Lab on a Chip, 22(13), 2578-2589) require multi-cell droplet compartmentalization, along with sorting of a sensor cell and the polypeptide-secreting cell. These dual-cell approaches within a single droplet generally have much lower throughput compared to single-cell droplet systems. Additionally, these platforms present technical complexity to be able to sort and select for droplets containing multiple cells. In contrast, our approach enables the recovery of the polypeptide-secreting cell linked with a selection marker associated with the activity of the antibody, enabling facile selection of cells that show desired activity.

Some alternative published approaches may screen secreted polypeptides for the interruption of receptor binding as a proxy signal for polypeptide activity, including virus neutralization (e.g., blocking ACE2 binding to the SARS-CoV-2 fusion protein (see e.g., Shiakolas, A. R., Kramer, K. J., Johnson, N. V., Wall, S. C., Suryadevara, N., Wrapp, D., Periasamy, S., Pilewski, K. A., Raju, N., Nargi, R., Sutton, R. E., Walker, L. M., Setliff, I., Crowe, J. E., Bukreyev, A., Carnahan, R. H., McLellan, J. S. & Georgiev, I. S. Efficient discovery of SARS-CoV-2-neutralizing antibodies via B cell receptor sequencing and ligand blocking. Nat Biotechnol (2022). doi:10.1038/s41587-022-01232-2).

However, a screen for receptor binding inhibition does not screen for neutralization directly, and there are many antibodies that will be missed from a selection round when screening for the interruption of receptor binding. Furthermore, screening for ligand blocking is unable to efficiently select for agonist antibodies. In contrast, herein we demonstrate the ability to screen for neutralization and for agonist antibodies directly using the current technology.

As disclosed herein, it is highly advantageous to implement high-throughput assays using single cells, rather than multiple cells inside droplets, for enhanced throughput and simplicity for the assay. Additionally, it is advantageous to be able to sort single cells, rather than sorting droplets, because single cells can be sorted using a broader range of cellular equipment (e.g., many different types of FACS machines available from multiple different vendors), whereas droplet sorting often requires specialized and/or custom equipment to implement.

Some alternative approaches perform binding assay screens for polypeptide secreted cells inside droplets (see e.g., Gérard, A., Woolfe, A., Mottet, G., Reichen, M., Castrillon, C., Menrath, V., Ellouze, S., Poitou, A., Doineau, R., Briseno-Roa, L., Canales-Herrerias, P., Mary, P., Rose, G., Ortega, C., Delincé, M., Essono, S., Jia, B., Iannascoli, B., Goff, O. R.-L., Kumar, R., Stewart, S. N., Pousse, Y., Shen, B., Grosselin, K., Saudemont, B., Sautel-Caillé, A., Godina, A., McNamara, S., Eyer, K., Millot, G. A., Baudry, J., England, P., Nizak, C., Jensen, A., Griffiths, A. D., Bruhns, P. & Brenan, C. High-throughput single-cell activity-based screening and sequencing of antibodies using droplet microfluidics. Nature Biotechnology 1-7 (2020). doi:10.1038/s41587-020-0466-7), for example, plasma cells secreting antibody. However, these technologies require droplet-based sorting which is complicated and inefficient, and the use of plasma cells prevents most neutralization assays and selections for agonist or antagonist molecules against membrane proteins like GPCRs. In contrast, our approach allows sorting of cells directly using standard FACS equipment, and is flexible for a broad range of membrane protein-based selections and viral neutralization assays that show major advantages compared with the technologies described in the prior art.

The procedures described here could be implemented with sorting cell droplets on a microfluidic chip as one variant of the procedure (e.g., sorting droplets before breaking the emulsions and collecting the cells), if desired.

In one aspect of the current disclosure, screening methods are provided. In some embodiments, the screening methods comprise: (a) detecting the presence and/or level of expression of a reporter molecule in a single, isolated, genetically engineered cell, wherein the cell presents a cell surface protein; and wherein the cell is engineered to: (i) secrete a heterologous test polypeptide; and (ii) express a reporter molecule if the test polypeptide activates the cell surface protein.

As used herein, “presents a cell surface protein” refers to the cell of interest having the cell surface protein localized to the cell surface. Localization of the cell surface protein may depend on the unique molecular properties of the cell surface protein itself. In addition, the localization of the cell surface protein may be required for function of the protein. In some embodiments, the cell surface protein is an integral membrane protein. In some embodiments, the cell surface protein is localized to the cell surface by a glycosylphosphatidylinositol (GPI) moiety. In some embodiments, the cell surface protein is capable of transducing a signal across the cell membrane into the cell. In other embodiments, the cell surface protein is present to allow entry of a test reagent, which may, e.g., comprise a reporter molecule. In some embodiments, the cell surface protein is expressed by the cell and is then localized to the cell membrane. In other embodiments, the cell surface protein is delivered to the cell by means known in the art, e.g., exosomes, microvesicles, liposomes, etc.

As used herein, “cell surface protein” is any cell surface-associated protein or polypeptide. In some embodiments, the cell surface protein is a protein for a ligand that is capable of transducing a signal inside the cell upon receptor ligation. Thus, in some embodiments, a cell surface protein comprises a cell surface receptor.

As used herein, “detecting” refers to acquiring information provided by one or more reporters in the cell. Accordingly, in some embodiments, detecting may be performed by an automated apparatus, e.g., a flow cytometer, fluorometer, luminometer, microscope, digital camera, plate reader, etc. or by the human eye. In other embodiments, detecting is performed using a technique related to the sequencing of nucleic acids, e.g., sanger sequencing, next generation sequencing (NGS), single-cell RNA sequencing (scRNA-seq) etc.

As used herein, “reporter molecule” refers to a molecule that is expressed by a cell of interest that indicates a particular molecular state of the cell. For example, in some embodiments, cell lines are engineered to provide a signal (e.g., express a reporter molecule) in response to receptor agonism or antagonism. Therefore, the reporter molecule indicates the state of the cell, i.e., that the receptor of interested has been ligated or has been prevented from being ligated. Exemplary reporter molecules include, but are not limited to, fluorescent proteins, luminescent proteins, enzymes, tagged proteins, nucleic acid sequences.

Exemplary fluorescent proteins include, but are not limited to the molecules provided below, and functional variants thereof:

| Green fluorescent protein (GFP), which has the sequence: | |
|---|---|
| (SEQ ID NO: 1) | |
| MSKGEELFTG VVPILVELDG DVNGHKFSVS GEGEGDATYG KLTLKFICTT GKLPVPWPTL | 60 |
| VTTFSYGVQC FSRYPDHMKQ HDFFKSAMPE GYVQERTIFF KDDGNYKTRA EVKFEGDTLV | 120 |
| NRIELKGIDF KEDGNILGHK LEYNYNSHNV YIMADKQKNG IKVNFKIRHN IEDGSVQLAD | 180 |
| HYQQNTPIGD GPVLLPDNHY LSTQSALSKD PNEKRDHMVL LEFVTAAGIT HGMDELYK | |
| Red fluorescent protein (RFP), which has the sequence: | |
| (SEQ ID NO: 2) | |
| MRGSHHHHHH GSAHGLTDDM TMHFRMEGCV DGHKFVIEGN GNGNPFKGKQ FINLCVIEGG | 60 |
| PLPFSEDILS AAFXNRLFTE YPEGIVDYFK NSCPAGYTWH RSFRFEDGAV CICSADITVN | 120 |
| VRENCIYHES TFYGVNFPAD GPVMKKMTTN WEPSCEKIIP INSQKILKGD VSMYLLLKDG | 180 |
| GRYRCQFDTI YKAKTEPKEM PDWHFIQHKL NREDRSDAKN QKWQLIEHAI ASRSALP | |
| Yellow fluorescent protein (YFP), which has the sequence: | |
| (SEQ ID NO: 3) | |
| KGEELFTGVV PILVELDGDV NGHKFSVSGE GEGDATYGKL TLKFICTTGK LPVPWPTLVT | 60 |
| TFXLQCFARY PDHMKRHDFF KSAMPEGYVQ ERTIFFKDDG NYKTRAEVKF EGDTLVNRIE | 120 |
| LKGIDFKEDG NILGHKLEYN YNSHNVYIMA DKQKNGIKVN FKIRHNIEDG SVQLADHYQQ | 180 |
| NTPIGDGPVL LPDNHYLSYQ SALSKDPNEK RDHMVLLEFV TAAGI | |
| Blue fluorescent protein (BFP), which has the sequence: | |
| (SEQ ID NO: 4) | |
| MSKGEELFTG VVPILVELDG DVNGHKFSVS GEGEGDATYG KLTLKFICTT GKLPVPWPTL | 60 |
| VTTFXVQCFS RYPDHMKRHD FFKSAMPEGY VQERTIFFKD DGNYKTRAEV KFEGDTLVNR | 120 |
| IELKGIDFKE DGNILGHKLE YNFNSHNVYI MADKQKNGIK VNFKIRHNIE DGSVQLADHY | 180 |
| QQNTPIGDGP VLLPDNHYLS TQSALSKDPN EKRDHMVLLE FVTAAGITHG MDELYK | |
| Cyan fluorescent protein (CFP), which has the sequence: | |
| (SEQ ID NO: 5) | |
| MVSKGEELFT GWPILVELD GDVNGHKFSV SGEGEGDATY GKLTLKFICT TGKLPVPWPT | 60 |
| LVTTLXVQCF ARYPDHMKQH DFFKSAMPEG YVQERTIFFK DDGNYKTRAE VKFEGDTLVN | 120 |
| RIELKGIDFK EDGNILGHKL EYNAISDNVY ITADKQKNGI KANFKIRHNI EDGSVQLADH | 180 |
| YQQNTPIGDG PVLLPDNHYL STQSALSKDP NEKRDHMVLL EFVTAAGITL GMDELYK | |
| Or | |
| (SEQ ID NO: 6) | |
| MVSKGEELFT GVVPILVELD GDVNGHKFSV SGEGEGDATY GKLTLKFICT TGKLPVPWPT | 60 |
| LVTTLXVQCF SRYPDHMKQH DFFKSAMPEG YVQERTIFFK DDGNYKTRAE VKFEGDTLVN | 120 |
| RIELKGIDFK EDGNILGHKL EYNYISHNVY ITADKQKNGI KANFKIRHNI EDGSVQLADH | 180 |
| YQQNTPIGDG PVLLPDNHYL STQSALSKDP NEKRDHMVLL EFVTAAGITL GMDELYK | |
| mCherry, which has the sequence: | |
| (SEQ ID NO: 7) | |
| MVSKGEEDNM AIIKEFMRFK VHMEGSVNGH EFEIEGEGEG RPYEGTQTAK LKVTKGGPLP | 60 |
| FAWDILSPQF MYGSKAYVKH PADIPDYLKL SFPEGFKWER VMNFEDGGVV TVTQDSSLQD | 120 |
| GEFIYKVKLR GTNFPSDGPV MQKKTMGWEA SSERMYPEDG ALKGEIKQRL KLKDGGHYDA | 180 |
| EVKTTYKAKK PVQLPGAYNV NIKLDITSHN EDYTIVEQYE RAEGRHSTGG MDELYK | |

Exemplary luminescent proteins include, but are not limited to:

| Renilla luciferase, which has the sequence: | |
|---|---|
| (SEQ ID NO: 8) | |
| MTSKVYDPEL RKRMITGPOW WARCKQMNVL DSFINYYDSE KHAENAVIFL HGNAASSYLW | 60 |
| RHVVPHVEPV ARCIIPDLIG MGKSGKSGNG SYRLLDHYKY LTEWFKHLNL PKKIIFVGHD | 120 |
| WGACLAFHYC YEHQDRIKAV VHAESVVDVI ESWDEWPDIE EDIALIKSEE GEKMVLENNF | 180 |
| FVETMLPSKI MRKLEPEEFA AYLEPFKEKG EVRRPTLSWP REIPLVKGGK PDVVEIVRNY | 240 |
| NAYLRASHDL PKMFIESDPG FFSNAIVEGA KKFPNTEFVK VKGLHFSQED APDEMGNYIK | 300 |
| SFVERVLKNE Q | |
| Firefly (Photimus pyralis) luciferase, which has the sequence: | |
| (SEQ ID NO: 9) | |
| MEDAKNIKKG PAPFYPLEDG TAGEQLHKAM KRYALVPGTI AFTDAHIEVN ITYAEYFEMS | 60 |
| VRLAEAMKRY GLNTNHRIVV CSENSLQFFM PVLGALFIGV AVAPANDIYN ERELLNSMNI | 120 |
| SQPTVVFVSK KGLQKILNVQ KKLPIIQKII IMDSKTDYQG FQSMYTFVTS HLPPGFNEYD | 180 |
| FVPESFDRDK TIALIMNSSG STGSPKGVAL PHRTACVRFS HARDPIFGNQ IIPDTAILSV | 240 |
| VPFHHGFGMF TTLGYLICGF RVVLMYRFEE ELFLRSLQDY KIQSALLVPT LFSFFAKSTL | 300 |
| IDKYDLSNLH EIASGGAPLS KEVGEAVAKR FHLPGIRQGY GLTETTSAIL ITPEGDDKPG | 360 |
| AVGKVVPFFE AKVVDLDTGK TLGVNQRGEL CVRGPMIMSG YVNDPEATNA LIDKDGWLHS | 420 |
| GDIAYWDEDE HFFIVDRLKS LIKYKGCQVA PAELESILLQ HPNIFDAGVA GLPGDDAGEL | 480 |
| PAAVVVLEHG KTMTEKEIVD YVASQVTTAK KLRGGVVFVD EVPKGLTGKL DARKIREILI | 540 |
| KAKKGGKSKL | |

As used herein, “expression” refers to either the transcription of a nucleic acid comprising DNA into RNA or the translation of said RNA into a protein or polypeptide, or both the transcription of DNA into RNA and translation of said RNA into a protein or polypeptide.

The methods disclosed herein utilize an efficient single-cell platform that allows for rapid and high-throughput testing of candidate molecules. Thus, as used herein “single, isolated cell” refers to a cell that is physically separated from other cells in a reaction vessel, e.g., a multi-well plate, a microchip, a microfluidics chip, a Nanopen™, and the like.

The methods, compositions, systems, and kits of the instant disclosure utilize “genetically engineered cells”. As used herein, “genetically engineered”, or grammatical variations thereof, refers to the cell possessing one or more genetic modifications made by the hand of man. Such modifications comprise, for example, expression of an introduced or exogenous nucleic acid. Methods of introducing exogenous nucleic acids are known in the art including, but not limited to, transfection, lipofection, viral transduction, e.g., retroviral, lentiviral, or adenoviral transduction. In some embodiments, genetically engineered cells comprise nucleic acids that are integrated into the genome of the cell, while in other embodiments, genetically engineered cells comprise nucleic acids that are contained in episomes.

In some embodiments, genetically engineered cells comprise nucleic acids which encode genes of interest operably controlled by one or more promoters or one or more enhancer sequences. In some embodiments, the promoters may have constitutive activity, i.e., the promoters continuously direct transcription of the nucleic acid under its control. Exemplary constitutive promoters include but are not limited to the cytomegalovirus (CMV) promoter and elongation factor 1α (EF1a) promoter. In other embodiments, the one or more promoters are inducible, meaning that they respond to addition of another molecule. Exemplary inducible promoters include tetracycline inducible promoters, cumate inducible promoters, and estrogen receptor-based tamoxifen inducible promoters. In some embodiments, promoters are “strong” promoters, with relatively high levels of expression of the downstream sequence. In some embodiments, promoters are “weak” promoters, with relatively low levels of expression of the downstream sequence. By way of example but not by way of limitation, the mammalian CMV promoter is generally considered to be a strong promoter by those skilled in the art.

In some embodiments, the methods of the present disclosure use a single cell as source of expression of both a protein of interest “cell surface protein”, and a potential ligand of interest, referred to as a “heterologous test peptide”. In some embodiments, the genetically modified cells express a reporter in response to successful ligation, and in some examples, downstream signaling, of the receptor of interest by the heterologous test peptide. Each cell to be screened is engineered to express a different potential ligand for the receptor of interest, in addition to the receptor itself, and the reporter molecule that indicates ligation of the receptor. Thus, screening of many such cells reveals a plurality of ligands for the receptor.

In some embodiments, the heterologous test peptide is an antibody, or an antigen binding fragment thereof, e.g., a single-chain variable fragment (scFv), nanobody, or Fab fragment. As used herein, “single-chain variable fragment (scFv)” refers to single immunoglobulin heavy and single immunoglobulin light chain fused by a linker. As used herein, a “nanobody” refers to a protein comprising a single monomeric variable antibody domain. “Fab” fragment refers to the antigen-binding region of an antibody.

In some embodiments, a ligand for a cell surface protein is known and the heterologous test polypeptide has a structure or sequence based on that of the known ligand.

In some embodiments the screening methods comprise: (a) detecting the presence and/or level of expression of a reporter molecule in a single, isolated, genetically engineered cell, wherein the cell presents a cell surface protein; and wherein the cell is engineered to: (i) secrete a heterologous test polypeptide; and (ii) express a reporter molecule if the test polypeptide does not activate the cell surface protein. Thus, in such embodiments, prevention of activation of the cell surface protein is revealed by the expression of the reporter molecule.

In some embodiments of the screening methods, the screening methods comprise: (a) contacting a single, isolated, genetically engineered cell with a test reagent, wherein the cell presents a cell surface protein, and wherein the cell is engineered to: (i) secrete a heterologous test polypeptide; and (ii) express a reporter molecule if one of the test polypeptide or the test reagent activates the cell surface protein; (b) detecting the presence and/or level of expression of the reporter molecule.

In some embodiments, the test reagent is a ligand of a cell surface protein, and the heterologous test polypeptide is a potential agonist or antagonist of the cell surface protein. In other embodiments, the test reagent is an antagonist or an agonist of the cell surface protein, and the heterologous test polypeptide is a potential ligand of the cell surface protein.

In some embodiments, the screening methods comprise: (a) contacting a single, isolated, genetically engineered cell with a test reagent comprising a reporter molecule, wherein the cell presents a cell surface protein; wherein the test reagent is capable of binding the cell surface protein presented by the cell, forming a reagent-protein complex, and wherein the test reagent gains entry into the cell when the reagent-protein complex is formed; wherein the cell is engineered to: (i) secrete a heterologous test polypeptide; (b) detecting the presence and/or level of expression of the reporter molecule in the cells.

In some embodiments, the test reagent is an infectious agent, or is derived from an infectious agent. In some embodiments, the test reagent is a virus, or is derived from a virus. Exemplary, non-limiting viruses include, for example, Coronavirus A, B, C, D, flaviviruses, lentiviruses, influenza A, B, C, or D viruses, Epstein-Barr virus, herpes simplex virus, cytomegalovirus, respiratory syncytial virus, Ebola virus, Marburg virus, Dengue virus. In some embodiments, the test reagent is, or is derived from, human immunodeficiency virus (HIV), yellow fever virus, severe acute respiratory syndrome coronavirus-2 (SARS-CoV-2), Epstein-Barr virus, herpes simplex virus, cytomegalovirus, respiratory syncytial virus, Ebola virus, Marburg virus, or Dengue virus. In some embodiments, the test reagent is a pseudovirus. As used herein, “pseudovirus” refers to a replication incompetent virus, or viral-like particle, often based on retroviruses, lentiviruses, e.g., HIV, or vesicular stomatitis virus, which additionally comprise a key viral factor from another virus, e.g., SARS-CoV-2 surface glycoprotein (spike protein). Thus, the risk of infection to researchers using the pseudovirus is mitigated, compared to the use of wild type virus, while being useful as a tool for the discovery of novel neutralization agents against the wild type virus, as in the methods, compositions, and kits disclosed herein. In some embodiments, the virus is capable of infecting a mammal, a fish, an avian, a plant, an insect, a yeast, or a bacterium.

In some embodiments, the test reagent comprises a reporter molecule. Therefore, once the test agent comprising the reporter molecule binds the cell surface protein and gains entry into the cell, the cell comprises the reporter molecule.

In some embodiments, the cell surface protein is a receptor that is required for complexing with the test reagent and catalyzing entry of the test reagent into the cell. In some embodiments the cell surface protein is a receptor for a ligand that is capable of transducing a signal inside the cell upon receptor ligation

Exemplary cell surface proteins include, but are not limited to:

| Human angiotensin converting enzyme 2 (hACE-2), which has the amino acid | |
|---|---|
| sequence: | |
| (SEQ ID NO: 10) | |
| MSSSSWLLLS LVAVTAAQST IEEQAKTFLD KFNHEAEDLF YQSSLASWNY NTNITEENVQ | 60 |
| NMNNAGDKWS AFLKEQSTLA QMYPLQEIQN LTVKLQLQAL QQNGSSVLSE DKSKRLNTIL | 120 |
| NTMSTIYSTG KVCNPDNPQE CLLLEPGLNE IMANSLDYNE RLWAWESWRS EVGKQLRPLY | 180 |
| EEYVVLKNEM ARANHYEDYG DYWRGDYEVN GVDGYDYSRG QLIEDVEHTF EEIKPLYEHL | 240 |
| HAYVRAKLMN AYPSYISPIG CLPAHLLGDM WGRFWTNLYS LTVPFGQKPN IDVTDAMVDQ | 300 |
| AWDAQRIFKE AEKFFVSVGL PNMTQGFWEN SMLTDPGNVQ KAVCHPTAWD LGKGDFRILM | 360 |
| CTKVTMDDFL TAHHEMGHIO YDMAYAAQPF LLRNGANEGF HEAVGEIMSL SAATPKHLKS | 420 |
| IGLLSPDFQE DNETEINFLL KQALTIVGTL PFTYMLEKWR WMVFKGEIPK DQWMKKWWEM | 480 |
| KREIVGVVEP VPHDETYCDP ASLFHVSNDY SFIRYYTRTL YQFQFQEALC QAAKHEGPLH | 540 |
| KCDISNSTEA GQKLFNMLRL GKSEPWTLAL ENVVGAKNMN VRPLLNYFEP LFTWLKDQNK | 600 |
| NSFVGWSTDW SPYADQSIKV RISLKSALGD KAYEWNDNEM YLFRSSVAYA MRQYFLKVKN | 660 |
| QMILFGEEDV RVANLKPRIS FNFFVTAPKN VSDIIPRTEV EKAIRMSRSR INDAFRLNDN | 720 |
| SLEFLGIQPT LGPPNQPPVS IWLIVFGVVM GVIVVGIVIL IFTGIRDRKK KNKARSGENP | 780 |
| YASIDISKGE NNPGFQNTDD VQTSF | |
| Human programmed cell death 1 protein (PD-1), which has the sequence: | |
| (SEQ ID NO: 11) | |
| MEDAKNIKKG PAPFYPLEDG TAGEQLHKAM KRYALVPGTI AFTDAHIEVN ITYAEYFEMS | 60 |
| VRLAEAMKRY GLNTNHRIVV CSENSLQFFM PVLGALFIGV AVAPANDIYN ERELLNSMNI | 120 |
| SQPTVVFVSK KGLQKILNVQ KKLPIIQKII IMDSKTDYQG FQSMYTFVTS HLPPGFNEYD | 180 |
| FVPESFDRDK TIALIMNSSG STGSPKGVAL PHRTACVRFS HARDPIFGNQ IIPDTAILSV | 240 |
| VPFHHGFGMF TTLGYLICGF RVVLMYRFEE ELFLRSLQDY KIQSALLVPT LFSFFAKSTL | 300 |
| IDKYDLSNLH EIASGGAPLS KEVGEAVAKR FHLPGIRQGY GLTETTSAIL ITPEGDDKPG | 360 |
| AVGKVVPFFE AKVVDLDTGK TLGVNQRGEL CVRGPMIMSG YVNDPEATNA LIDKDGWLHS | 420 |
| GDIAYWDEDE HFFIVDRLKS LIKYKGCQVA PAELESILLQ HPNIFDAGVA GLPGDDAGEL | 480 |
| PAAVVVLEHG KTMTEKEIVD YVASQVTTAK KLRGGVVFVD EVPKGLTGKL DARKIREILI | 540 |
| KAKKGGKSKL | |
| Human cytotoxic T lymphocyte protein 4 (CTLA-4), which has the sequence: | |
| (SEQ ID NO: 12) | |
| MACLGFQRHK AQLNLATRTW PCTLLFFLLF IPVFCKAMHV AQPAVVLASS RGIASFVCEY | 60 |
| ASPGKATEVR VTVLRQADSQ VTEVCAATYM MGNELTFLDD SICTGTSSGN QVNLTIQGLR | 120 |
| AMDTGLYICK VELMYPPPYY LGIGNGTQIY VIDPEPCPDS DFLLWILAAV SSGLFFYSFL | 180 |
| LTAVSLSKML KKRSPLTTGV YVKMPPTEPE CEKQFQPYFI PIN | |
| Human 4-1BB, which has the sequence | |
| (SEQ ID NO: 13) | |
| MGNSCYNIVA TLLLVLNFER TRSLQDPCSN CPAGTFCDNN RNQICSPCPP NSFSSAGGQR | 60 |
| TCDICRQCKG VFRTRKECSS TSNAECDCTP GFHCLGAGCS MCEQDCKQGQ ELTKKGCKDC | 120 |
| CFGTFNDQKR GICRPWTNCS LDGKSVLVNG TKERDVVCGP SPADLSPGAS SVTPPAPARE | 180 |
| PGHSPQIISF FLALTSTALL FLLFFLTLRF SVVKRGRKKL LYIFKQPFMR PVQTTQEEDG | 240 |
| CSCRFPEEEE GGCEL | |
| Human hepatitis A virus cellular receptor 2 (TIM-3), which has the sequence: | |
| (SEQ ID NO: 14) | |
| MFSHLPFDCV LLLLLLLLTR SSEVEYRAEV GQNAYLPCFY TPAAPGNLVP VCWGKGACPV | 60 |
| FECGNVVLRT DERDVNYWTS RYWLNGDFRK GDVSLTIENV TLADSGIYCC RIQIPGIMND | 120 |
| EKFNLKLVIK PAKVTPAPTR QRDFTAAFPR MLTTRGHGPA ETQTLGSLPD INLTQISTLA | 180 |
| NELRDSRLAN DLRDSGATIR IGIYIGAGIC AGLALALIFG ALIFKWYSHS KEKIQNLSLI | 240 |
| SLANLPPSGL ANAVAEGIRS EENIYTIEEN VYEVEEPNEY YCYVSSRQQP SQPLGCRFAM | 300 |
| P | |
| Human lymphocyte activation gene 3 (LAG3), which has the sequence: | |
| (SEQ ID NO: 15) | |
| MWEAQFLGLL FLQPLWVAPV KPLQPGAEVP VVWAQEGAPA QLPCSPTIPL QDLSLLRRAG | 60 |
| VTWQHQPDSG PPAAAPGHPL APGPHPAAPS SWGPRPRRYT VLSVGPGGLR SGRLPLQPRV | 120 |
| QLDERGRQRG DFSLWLRPAR RADAGEYRAA VHLRDRALSC RLRLRLGQAS MTASPPGSLR | 180 |
| ASDWVILNCS FSRPDRPASV HWFRNRGQGR VPVRESPHHH LAESFLFLPQ VSPMDSGPWG | 240 |
| CILTYRDGFN VSIMYNLTVL GLEPPTPLTV YAGAGSRVGL PCRLPAGVGT RSFLTAKWTP | 300 |
| PGGGPDLLVT GDNGDFTLRL EDVSQAQAGT YTCHIHLQEQ QLNATVTLAI ITVTPKSFGS | 360 |
| PGSLGKLLCE VTPVSGQERF VWSSLDTPSQ RSFSGPWLEA QEAQLLSQPW QCQLYQGERL | 420 |
| LGAAVYFTEL SSPGAQRSGR APGALPAGHL LLFLILGVLS LLLLVTGAFG FHLWRRQWRP | 480 |
| RRFSALEOGI HPPOAQSKIE ELEQEPEPEP EPEPEPEPEP EPEQL | |

hACE-2 is required for SARS-CoV-2 entry into cells, while TMPRSS2 potentiates the entry of the virus into the cell. Thus, in some embodiments, cells of the instant disclosure may comprise both hACE-2 and TMPRSS2.

| Human transmembrane serine protease 2 (TMPRSS2), which has the sequence | |
|---|---|
| (SEQ ID NO: 16) | |
| MALNSGSPPA IGPYYENHGY QPENPYPAQP TVVPTVYEVH PAQYYPSPVP QYAPRVLTQA | 60 |
| SNPVVCTQPK SPSGTVCTSK TKKALCITLT LGTFLVGAAL AAGLLWKFMG SKCSNSGIEC | 120 |
| DSSGTCINPS NWCDGVSHCP GGEDENRCVR LYGPNFILQV YSSQRKSWHP VCQDDWNENY | 180 |
| GRAACRDMGY KNNFYSSQGI VDDSGSTSFM KLNTSAGNVD IYKKLYHSDA CSSKAVVSLR | 240 |
| CIACGVNLNS SRQSRIVGGE SALPGAWPWQ VSLHVQNVHV CGGSIITPEW IVTAAHCVEK | 300 |
| PLNNPWHWTA FAGILRQSFM FYGAGYQVEK VISHPNYDSK TKNNDIALMK LQKPLTFNDL | 360 |
| VKPVCLPNPG MMLQPEQLCW ISGWGATEEK GKTSEVLNAA KVLLIETQRC NSRYVYDNLI | 420 |
| TPAMICAGFL QGNVDSCQGD SGGPLVTSKN NIWWLIGDTS WGSGCAKAYR PGVYGNVMVF | 480 |
| TDWIYRQMRA DG | |

Programmed cell death protein 1 (PD-1) is a transmembrane protein which contains immunoreceptor tyrosine-based inhibitory motifs (TUNIS) and an immunoreceptor tyrosine-based switch motif, which suggests that PD-1 negatively regulates T-cell receptor TCR signals. Agents which block PD-1 signaling, therefore, increase T cell effector function and have been used successfully to treat cancer.

Human cytotoxic T lymphocyte protein 4 (CTLA-4) is a transmembrane protein that binds to the co-stimulatory molecules CD80 and CD86 on antigen presenting cells (APCs) and transduces co-inhibitory signals to the T cell. Agents which block CTLA-4 signaling, therefore, increase T cell effector function and have been used successfully to treat cancer.

4-1BB (CD137, or TNFRSF9) is a membrane protein that acts to stimulate the effector function of T cells. Therefore, agents that modulate 4-1BB signaling may be useful for the treatment of human disease. For example, agents that stimulate 4-1BB may be useful to activate tumor infiltrating lymphocytes to destroy cancer cells, while agents that antagonize 4-1BB signaling may be useful to prevent autoimmunity or treat transplant-related symptoms in humans.

Human hepatitis A virus cellular receptor 2 (TIM-3) is a transmembrane protein that acts as an inhibitory molecule in T cells. Therefore, agents that reduce or block TIM-3 signaling may be useful in cancer immunotherapy.

Human lymphocyte activation gene 3 (LAG3) is a is a transmembrane protein that acts as an inhibitory molecule in T cells. Therefore, agents that reduce or block LAG3 signaling may be useful in cancer immunotherapy.

In some embodiments, the heterologous test peptide secreted by cells comprises any protein that may neutralize the virus or alter cell function to prevent viral infection. Exemplary secreted proteins may include interferon variants, griffithsin, peptides, receptor traps (e.g., soluble ACE2 variants for SARS-CoV-2, or soluble CD4 variants for HIV-1).

In some embodiments of the screening methods, the methods further comprise amplifying and/or sequencing the nucleic acids encoding the heterologous test polypeptide. Thus, in some embodiments, cells that express the reporter molecule may be separated from those not expressing the reporter molecule by methods known in the art, e.g., fluorescence activated cell sorting (FACS), magnetic bead enrichment, and each group sequenced to produce libraries of sequences encoding heterologous test polypeptides associated with the expression, or lack of expression of the reporter in the given system.

In some embodiments, the reporter molecule comprises a nucleic acid sequence. In some embodiments, said nucleic acid sequence comprises a barcode sequence. As used herein, “barcode” or “barcode sequence” refers to a unique nucleotide sequence used to identify a particular condition, e.g., ligation of a cell surface protein. Barcode sequences suitably comprise sequences that are not found in the genome, transcriptome, exogenous expression vectors, etc. present in the cell in which the barcodes are expressed so as to be readily identifiable.

The present technology is not limited to a specific cell type or a specific cell line, and any suitable cell, including prokaryotic cells (e.g., bacterial), yeast, mammal, avian, fish, or plant cells may be used for both viral infection neutralization assays, and to test polypeptide-receptor activity (e.g., antibody, ligand, receptor, agonist, antagonist, etc.). Exemplary, non-limiting cell lines useful for the screening assays disclosed herein such as neutralization assays, include CHO, BHK, Cos-7 NS0, SP2/0, YB2/0, HEK293, HT-1080, Huh-7, PER.C6, and variants thereof, or others. In some embodiments, B cell lines may be used, for example Raji, ARH-77, MOPC-315, MOPC-21, or others.

By way of example, in some embodiments, insect cells may be used, along with a reporter compatible with insect cells. In some embodiments, the reporter may be induced by insect cell viruses. In some embodiments, bacterial cells may be used, along with a reporter compatible with bacterial cells. In some embodiments, the reporter may be induced by bacteriophage infection. In some embodiments, plant cells may be used, along with a reporter compatible with plant cells. In some embodiments, the reporter may be induced by plant cell viruses. In some embodiments, mammalian cells may be used with a reporter compatible with mammalian cells, e.g., expression of fluorescent markers, enzymes, tagged proteins, or nucleic acids. In some embodiments, the mammalian cells are human cells. In some embodiments, the reporter may be induced by mammalian cell viruses.

In some embodiments, the assay readout may be a fluorophore expression. In some embodiments, the assay readout may be based on a Next Generation Sequencing (“NGS”) NGS-based signal or integrated NGS barcode. In some embodiments, the assay readout may be cell growth or cell death.

In some examples, a selectable marker may be used to select for cells transformed with nucleic acids encoding antibody and/or viral entry receptors.

As used herein, “selectable marker” refers to any molecule which permits the selection of a cell expressing the desired nucleic acid comprising nucleic acids encoding the selectable marker and a nucleic acid of interest. For example, in one embodiment, a cell of the instant disclosure expresses a nucleic acid comprising a nucleic acid encoding an antibody and encoding a fluorescent molecule, e.g., a fluorescent protein, (the selectable marker). Therefore, in the previous example, cells that are expressing the desired nucleic acid may be separated from cells not expressing the nucleic acid by use of methods known in the art to separate cells expressing a fluorescent molecule, e.g., fluorescence activated cell sorting (FACS). Other methods of separating cells expressing a selectable marker are known in the art including, but not limited to, antibody and magnetic bead separation. In some embodiments, the selectable marker confers a survival advantage to the cells expressing the nucleic acid of interest. For example, in some embodiments, the selectable marker confers resistance to antibiotics, e.g., blasticidin, Hygromycin B, puromycin, zeocin, G418/Geneticin, or⁽¹⁾ others. Thus, treatment of cells with the antibiotic for which molecules conferring resistance are encoded on the nucleic acid of interest, selects cells expressing the nucleic acid of interest and, therefore, acts as a selectable marker.

Thus, in some embodiments, a reporter comprises a selectable marker. However, though a reporter may, in some embodiments, comprise a selectable marker, a reporter functions to indicate to one of skill in the art practicing the disclosed methods, using the disclosed compositions or kits, that there is a change in the status of the cell in which the reporter is expressed, e.g., infection with a virus, presence of a cellular signaling event, lack of a cellular signaling event, etc.

In some embodiments, a selectable marker expressed by the cells may be used that enables selection for optimal protein or peptide function from a library of protein or peptide variants. In some embodiments, the selectable marker of secreted protein function may be a fluorescent protein not normally expressed in the cell line, including but not limited to green fluorescent protein (GFP), red fluorescent protein (RFP), yellow fluorescent protein (RFP), mCherry, blue fluorescent protein (BFP), cyan fluorescent protein (CFP) and others. In some embodiments, the selectable marker may induce expression of a surface protein for affinity-based selection, some examples might include CD19, CD4, CD34, and other surface proteins. In other embodiments, the selectable marker may include an enzyme that enables cell survival, including but not limited to apoptosis pathway genes, glutathione S-transferase, antibiotic resistance markers, Bleomycin, Adenosine deaminase, Xanthine-guanine phosphoribosyltransferase, or⁽¹⁾ others. In some embodiments, the selectable marker may be read as a result of Cre-lox or CRISPR gene activation resulting in chromosomal changes. In some embodiments, an integrase may be used to insert genes into the cells for cloning libraries. In other embodiments, stable cell pools may be used to generate libraries from transfected plasmids. In other embodiments, secreted protein libraries may be generated using an integrase. In other embodiments, secreted protein libraries may be generated using a transposase. In some embodiments, the readout of the assay may be based on sequencing of cell populations after screening. In some embodiments, that assay readout may involve the identification of DNA barcodes encoded by the antibodies and/or the virus or viral infection model as a unique identifier of the antibody or viral infection variant, respectively. In other embodiments, the readout of the assay may be based on fluorescent markers and sorting via flow cytometry.

In some embodiments, the heterologous test polypeptide may be an antibody variant. In some embodiments, the antibody may be of one or more of the following formats: IgG, IgM, IgA, Fab, ScFv, Fab2′. In some embodiments, the antibody may be a bispecific antibody. In other embodiments, the antibody may be a trispecific antibody. In some embodiments, the secreted proteins may comprise antibody native heavy:light^{(2,3,4)} pairs.

In some embodiments, the heterologous test polypeptides may comprise randomly paired heavy and light chains. In some embodiments, the antibody heavy:light expression may be on the same mRNA transcript. In other embodiments, the antibody heavy and light chains may be expressed on separate mRNAs. In some embodiments, a bidirectional promoter may be used in between the heavy and light⁽³⁾ chain mRNAs.

In some embodiments, the heterologous test polypeptides comprise antibodies found in antibody gene libraries derived from human patients developed by screening of native human immune libraries. In other embodiments, the antibody gene libraries may be derived from animal sources, including mouse, transgenic mouse, camellid, shark, non-human primate, guinea pig, or other animals. In some embodiments, the antibody gene libraries may be synthetically generated. In certain embodiments, the libraries may comprise synthetically generated libraries with introduced diversity (for example, via targeted mutagenesis, site-saturation mutagenesis, DNA shuffling, error-prone PCR, somatic hypermutation, or other diversity introducing mechanisms). In some embodiments, the protein library may be based on antibody genes with known activity. In some embodiments, the disclosed screening methods may be used to select for improved potency, selectivity, or breadth of diversified libraries derived from antibodies with known baseline activity. In some embodiments, a heterologous test polypeptide may be selected for its ability to agonize or antagonize cellular receptors expressed by any species, including but not limited to mouse, non-human primate, guinea pig, ferret, pig, and human.

In some embodiments, the heterologous test polypeptide, or heterologous test polypeptide library variants may have some baseline activity, and the functional screen described is used to improve its potency, selectivity, or breadth of activity. In some embodiments, the starting protein or peptide library may have uncharacterized activity, and the functional assays described herein are used to characterize the functional activities of variants in the protein or peptide library and select for desired functional variants.

In some embodiments, the cell may be engineered to introduce genetic diversity to the secreted polypeptides (heterologous test polypeptides) between selection rounds. Several mechanisms for introducing genetic diversity are known to individuals skilled in the art, including the expression of activation-induced cytidine deamidase (AID), expression of an error-prone polymerase, or the use of an orthogonal plasmid replication system.

In some embodiments, the heterologous test polypeptide expression promoters may be varied to modulate the secreted protein concentrations, where stronger promoters influence the secreted concentration. Weaker promoters may be used to enable more potent secreted protein selection. In some embodiments, the amount of time of protein secretion may be varied to similarly adjust secreted protein concentrations. In some embodiments, by way of example, a shorter incubation time prior to the addition of virus can provide a lower soluble polypeptide concentration in supernatant, thereby selecting for more potently active or protective secreted molecules.

In some embodiments, the functional assay resulting in a reporter (e.g., GFP expression) may derive from a virus infection, and the assay comprises a virus neutralization assay, wherein the heterologous polypeptide expressed and secreted by the cell is an antibody, or antigen binding fragment.

In some embodiments, the functional assay may comprise the binding and activation or signal transduction via a cellular receptor (for example, a G-protein coupled receptor, a T cell receptor, a chimeric antigen receptor, an apoptosis marker, an immunomodulator such as PD-1, LAG-3, TIM, 4-1BB, or others). In such embodiments, the functional assay may comprise a screen for secreted proteins that can activate the cellular receptor and induce signal transduction. The signal transduction event could be linked to any reporter (e.g., fluorescent protein expression, apoptosis markers, cell surface marker expression, Cre-Lox or CRISPR expression, or mRNA-based markers) that would enable readout of the secreted protein's functional effect on the desired cellular receptor activation. In some embodiments, the secreted protein may block the surface receptor and prevent its activation in the presence of activating moieties (e.g., a ligand naturally produced by the cell, engineered to be produced by the cell, or added to contact the cell), resulting in a functional readout, e.g., a reporter. In other embodiments, the secreted protein may directly activate the surface receptor.

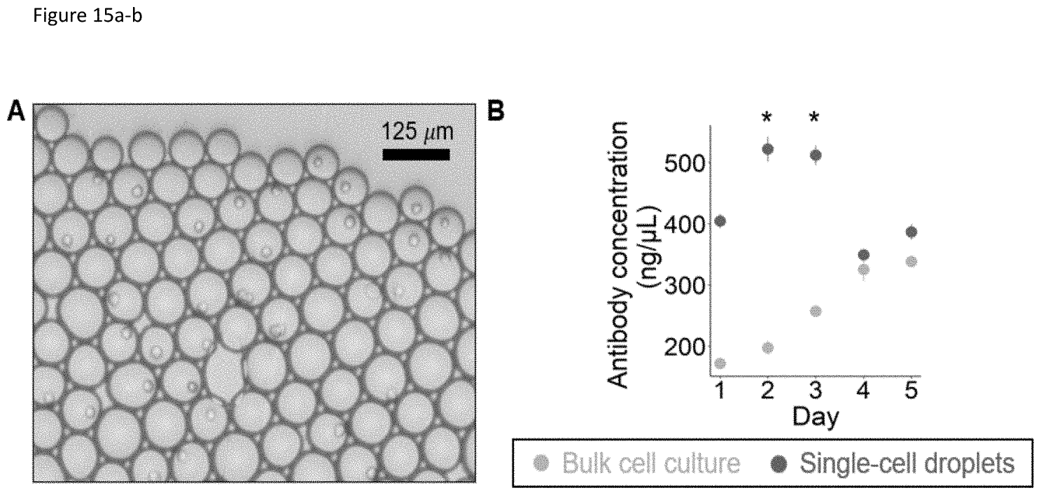

In some embodiments, single cells are isolated into compartments for functional screening of the secreted proteins. In some embodiments, the compartments may be 96- or 384 well plates. In some embodiments, the compartments may be printed^{(4,5)} microwells, open microchambers, or Nanopens™. Nanopens are cell-containing devices that include nanoliter-scale wells arranged in an arrays, and have been commercially available via the Berkeley Lights company. In other embodiments, the compartments may be emulsion⁽⁶⁾ droplets (See, for example, FIGS. 15, 16, 18, 28, and 29). In some embodiments, additional reagents may be added to the compartments after a certain amount of time has passed for the desired secreted test polypeptide to accumulate inside droplets. In well plates, reagent addition may occur by fluid addition. In printed^{(4,5)} microwells, open microchambers, or Nanopen™, reagent addition may be accomplished by washing or fluid flow near the unsealed compartment. In emulsion droplets, reagent addition may occur by droplet merger. In some embodiments, droplet merger may be accomplished by electrocoalescence (See Example 26, FIG. 29), printed pillar resistance, or other means of induced droplet fusion. In certain embodiments, the addition of reagents after the initial encapsulation of a library cell comprising a secreted protein variant may be unnecessary.

In some embodiments, the reagents added to the compartments may contain a virus or pseudovirus, in which case the assay may be a virus neutralization assay. In some embodiments, only a single virus or pseudovirus may be added. In other embodiments, multiple viruses or viral variants may be added. In some embodiments, the viruses or pseudoviruses may be barcoded with different selection markers to identify the infecting virus. In some embodiments, the viruses or pseudoviruses may be barcoded, tagged, or labeled with one or more different fluorescent markers, DNA barcodes, or cell surface proteins. In some embodiments, the virus or pseudovirus infection may cause cell death, and only cells encoding protective secreted proteins that neutralize the virus or pseudovirus can survive after the assay.

A longstanding challenge in antibody engineering and discovery is the need to identify agonistic or antagonistic antibodies against membrane proteins. Manipulating cellular behavior using membrane protein interactions is an important goal in modern medicine, including in cancer biology and in autoimmune disease treatments. Some examples of important membrane protein targets include the surface markers 4-1BB, OX40, PD-L1, PD-1, CTLA-4, LAG-3, G protein-coupled receptors (GPCR), and ion channels. Two of the biggest challenges to the discovery of antibodies targeting membrane proteins include: 1) the ability to express and purify soluble versions of the membrane-bound protein, because membrane proteins are non-native when expressed in a soluble format, and 2) it is technically complex to screen for the function of antibodies that bind to native, membrane-bound versions of the⁽⁷⁾ proteins, rather than simply screening for binding. The presently described approach for connecting together a secreted test protein expression in the same cell as membrane surface expression of target proteins elegantly addresses these two traditional challenges because there is no need to express and purify the membrane protein in a non-native solubilized format for screening, and also because the use of cell-based activation markers (such as fluorescence marker expression or luciferase expression) can provide a direct readout of the functional activity of the test protein that is secreted by a single cell. Thus, the approach described here for secreted protein analysis can be used for the important membrane targets, including surface proteins and receptors like 4-1BB, OX40, PD-L1, PD-1, CTLA-4, LAG-3, G protein coupled receptors (GPCR), and ion channels.

In some embodiments, the secreted protein activity may be an agonist or antagonist of receptor activity. In certain embodiments, a reagent, could be added to the compartments and may be e.g., a receptor agonist, for example PD-L1 for the PD-1 receptor. In other embodiments, the reagents added to the compartments may be receptor antagonists that prevent receptor activation upon binding. In some of these embodiments, receptor activation is linked to reporter expression, for example, fluorescent moiety expression to screen cells for their ability to secrete an antibody regulating protein receptor activity. Certain cell lines with receptor activation reporters comprising fluorescent signals, luciferase signals, or other signals to indicate receptor activity have been generated and may be used for this purpose, once they are suitably transformed with libraries of secreted proteins for analysis and selection.

The following are a set of assays that may utilize, or be utilized by, aspects of this application. The assays may be commercially available through various companies, such as the Promega company. An example of an assay used for the detection and/or characterization of membrane bound proteins and/or secreted proteins is the 4-1BB assay. 4-1BB (CD137/TNFRSF9), a member of the tumor necrosis factor receptor superfamily, is an inducible co-stimulatory receptor expressed on T cells, natural killer (NK) cells and innate immune cell populations. When present on the cell surface, 4-1BB interacts with 4-1BB ligand (4-1BBL) and induces subsequent cell proliferation and production of interferon gamma (IFNγ) and interleukin-2 (IL-2), particularly in T and NK cells. Another example of an assay used for the detection and/or characterization of membrane bound proteins and/or secreted proteins is the OX40 assay. The OX40 Bioassay is a bioluminescent cell-based assay that measures the potency and stability of ligands or agonist antibodies that can bind and activate OX40. OX40 (CD134/TNFRSF4), a member of the tumor necrosis factor (TNF) receptor superfamily, is a costimulatory receptor expressed primarily on activated T cells, and on neutrophils and natural killer (NK) cells to a lesser extent. When present on the cell surface, OX40 interacts with OX40 ligand (OX40L) and induces subsequent cell proliferation, survival and production of cytokines, particularly in T cells.

Another example of an assay used for the detection and/or characterization of membrane bound proteins and/or secreted proteins is the PD-1/PD-L1 assay. PD-1 is an immune inhibitory receptor expressed on activated T cells and B cells and plays a critical role in regulating immune responses to tumor antigens and autoantigens. Engagement of PD-1 by either of its ligands, PD-L1 or PD-L2, on an adjacent cell inhibits TCR signaling and TCR-mediated proliferation, transcriptional activation and cytokine production. Therapeutic antibodies and Fc fusion proteins designed to block the PD-1/PD-L1 interaction show promising results in clinical trials for the treatment of a variety of cancers. Another example of an assay used for the detection and/or characterization of membrane bound proteins and/or secreted proteins is the CTLA-4 assay. CTLA-4 (Cytotoxic T-lymphocyte-associated protein 4), also known as CD152, is an immune inhibitory receptor constitutively expressed on regulatory T cells (Tregs) and upregulated in activated T cells. CTLA-4 plays a critical role in regulating immune responses to tumor antigens and autoantigens. When CTLA-4 expression is upregulated on the surface of T cells, the T cells bind B7 with a higher avidity, and thus outcompete the positive co-stimulatory signal from CD28. In addition, engagement of CTLA-4 by either of its ligands, CD80 (B7-1) or CD86 (B7-2) on an adjacent antigen presenting cell (APC) inhibits CD28 co-stimulation of T cell activation, cell proliferation and cytokine production.

Another example of an assay used for the detection and/or characterization of membrane bound proteins and/or secreted proteins is the LAG-3/MHCII Blockade Bioassay assay. The LAG-3/MHCII Blockade Bioassay is a bioluminescent cell-based assay that measures potency and stability of antibodies and other biologics designed to block the interaction of LAG-3 with its best characterized ligand, major histocompatibility complex II (MHCII). LAG-3, also known as CD223, is an immune checkpoint receptor expressed on activated CD4+ and CD8+ T cells and natural killer (NK) cells. LAG-3 plays a critical role in regulating immune responses to tumor antigens and autoantigens. Engagement of LAG-3 by MHCII inhibits TCR signaling, cytokine production and proliferation of activated T cells. Therapeutic antibodies designed to block the LAG-3/MHCII interaction show promising results in clinical trials for the treatment of a variety of cancers.

Compositions

In one aspect of the current disclosure, compositions are provided. In some embodiments, the compositions comprise: a single, isolated, genetically engineered cell, wherein the cell presents a cell surface protein; and wherein the cell is engineered to: (i) secrete a heterologous test polypeptide; and (ii) express a reporter molecule if the test polypeptide activates the cell surface protein.

In some embodiments, the compositions comprise a single, isolated, genetically engineered cell, wherein the cell presents a cell surface protein; and wherein the cell is engineered to: (i) secrete a heterologous test polypeptide; and (ii) express a reporter molecule if the test polypeptide does not activate the cell surface protein.

In some embodiments, the compositions comprise a single, isolated, genetically engineered cell, and optionally a test reagent, wherein the cell presents a cell surface protein, and wherein the cell is engineered to: (i) secrete a heterologous test polypeptide; and (ii) express a reporter molecule if one of the test polypeptide or the test reagent activates the cell surface protein.

In some embodiments, the compositions comprise a single, isolated, genetically engineered cell, and optionally, a test reagent comprising a reporter molecule, wherein the cell presents a cell surface protein; wherein the test reagent is capable of binding the cell surface protein presented by the cell, forming a reagent-receptor complex, and wherein the test reagent gains entry into the cell when the reagent-receptor complex is formed; wherein the cell is engineered to: (i) secrete a heterologous test polypeptide.

In some embodiments, the cell comprises a mammalian cell, an insect cell, an avian cell, a yeast cell, a plant cell, or a bacterial cell. In some embodiments, the cell comprises a human cell.

In some embodiments, the cell surface protein comprises an endogenous receptor. In some embodiments, the cell is engineered to express the cell surface protein. In some embodiments, the cell surface protein comprises a heterologous protein.

In some embodiments, secretion of the test polypeptide is constitutive. In some embodiments, secretion of the test polypeptide is inducible.

In some embodiments, the single, isolated, genetically engineered cell is in a well of a multi-well plate. In some embodiments, the single, isolated, genetically engineered cell is in a chamber of a microchip. In some embodiments, the single, isolated, genetically engineered cell is in a microfluid droplet, such as an emulsion droplet. In some embodiments, the single, isolated, genetically engineered cell is in a Nanopen™.

In some embodiments, the reporter molecule comprises a fluorescent marker, an enzyme, a tagged protein, or a nucleic acid sequence. In some embodiments, the reporter molecule comprises a nucleic acid sequence, optionally a barcode sequence. In some embodiments, the reporter molecule comprises a fluorescent moiety.

In some embodiments, the heterologous test peptide comprises a variant of the receptor ligand. In some embodiments, the variant is derived from a library of ligand variants. In some embodiments, the heterologous test polypeptide comprises a potential receptor agonist or antagonist.

In some embodiments, the test reagent comprises an agonist or an antagonist of receptor activation. In some embodiments, the test reagent comprises the cell surface protein ligand, and the heterologous test polypeptide is derived from a library of potential agonists or antagonists of receptor activation.

In some embodiments, the heterologous test polypeptide comprises an antibody, a VHH (e.g., an antigen binding fragment of heavy chain only antibodies, as referred to as a nonobody) or antigen binding fragment thereof. In some embodiments, the antibody or antigen binding fragment is derived from a library of antibodies, or antigen binding fragments.

In some embodiments, the test reagent comprises a virus, and the cell surface protein comprises a component of viral entry into the cell. In some embodiments, the virus is on or more selected from Coronavirus A, B, C, or D, Flavivirus, Lentivirus, Influenza A, B, or C. In some embodiments, the virus selected from HIV, SARS-CoV-2, and Yellow Fever Virus. In some embodiments, the virus comprises a SARS-CoV-2 virus, and wherein the cell surface protein comprises a human angiotensin-converting enzyme 2 (hACE2). In some embodiments, the cell is engineered to express Transmembrane Serine Protease 2 (TMPRSS2).

In some embodiments, the cell is also engineered to introduce new gene diversity to the heterologous test polypeptide between selection rounds. Several mechanisms for introducing genetic diversity are known to individuals skilled in the art, including the expression of activation-induced cytidine deamidase (AID), expression of an error-prone polymerase, or the use of an orthogonal plasmid replication system.

Kits

In another aspect of the current disclosure, kits are provided. In some embodiments, the kits comprise: (a) a vector for the expression of a heterologous test polypeptide into a cell; (b) a vector encoding a reporter molecule, expression of which is activated if the heterologous test polypeptide activates a cell surface protein presented on the cell, optionally, wherein one or more of the vectors are expression vectors, or, optionally, wherein one or more of the vectors are integration vectors.

In some embodiments, the kits comprise: (a) a vector for the expression of a heterologous test polypeptide in a cell (b) a vector encoding a reporter molecule, expression of which is activated if the heterologous test polypeptide does not activate a cell surface protein, optionally, wherein one or more of the vectors are expression vectors, or, optionally, wherein one or more of the vectors are integration vectors.

In some embodiments, the kits comprise: (1) a test reagent; and (2) (a) a vector for the expression of a heterologous test polypeptide into a cell; (b) a vector encoding a reporter molecule, expression of which is activated if either the heterologous test polypeptide or test reagent activates a cell surface protein, optionally, wherein one or more of the vectors are expression vectors, or, optionally, wherein one or more of the vectors are integration vectors.

In some embodiments, the kits comprise: (1) a test reagent comprising a reporter molecule and (2) (a) a vector for expressing a test polypeptide in a cell, optionally, wherein the vector is an expression vector, or, optionally, wherein the vector is an integration vector.

In some embodiments, the kits additionally or alternatively comprise one or more vectors for the expression of (c) a cell surface protein. In some embodiments, the heterologous test polypeptide is operably linked to a promoter. In some embodiments, the promoter is a constitutive promoter. In some embodiments, the promoter is an inducible promoter. In some embodiments, the reporter molecule comprises one or more of a fluorescent marker and a barcode. In some embodiments, the reporter molecule is operably linked to an inducible promoter. In some embodiments, the test reagent comprises a virus or a pseudovirus. In some embodiments, the virus is selected from one or more of a Coronavirus A, B, C, or D, Flavivirus, Lentivirus, and Influenza A, B, or C. In some embodiments, the virus is selected from HIV, SARS-CoV-2, and Yellow Fever Virus. In some embodiments, the pseudovirus comprises a peptide, polypeptide, or protein derived from one or more of a Coronavirus A, B, C, or D, Flavivirus, Lentivirus, or Influenza A, B, or C. In some embodiments, the pseudovirus comprises a peptide, polypeptide, or protein derived from HIV, SARS-CoV-2, or Yellow Fever Virus. In some embodiments, the heterologous test peptide comprises an antibody, or a portion thereof. In some embodiments, the heterologous test peptide is a single chain variable fragment (scFv) or a nanobody.

In some embodiments, the kits comprise: (1) a vector for expressing a heterologous test polypeptide, (2) a genetically engineered cell comprising: (a) a nucleic acid encoding a reporter, expression of which is activated if the heterologous test polypeptide activates a cell surface protein.

In some embodiments, the kits comprise: (1) a vector for expressing a heterologous test polypeptide, (2) a genetically engineered cell comprising: (a) a nucleic acid encoding a reporter, expression of which is activated if the heterologous test polypeptide does not activate a cell surface protein.

In some embodiments, the kits comprise: (1) a vector for expressing a heterologous test polypeptide, (2) a genetically engineered cell comprising: (a) a nucleic acid encoding a reporter, expression of which is activated if the heterologous test polypeptide activates a cell surface protein, and optionally, (3) a test reagent.

In some embodiments, the kits comprise: (1) a vector for expressing a heterologous test polypeptide, (2) a genetically engineered cell (3) a test reagent comprising a reporter molecule, wherein the test reagent is capable of binding a cell surface protein presented by the cell, forming a reagent-receptor complex, and wherein the test reagent gains entry into the cell when the reagent-receptor complex is formed.

In some embodiments, the genetically engineered cell further comprises: (c) a nucleic acid encoding a heterologous cell surface protein.

As used herein, “expression vector” refers to a vector that is used to express a nucleic acid sequence of interest encoded on the vector. In some embodiments, the expression vector expresses the nucleic acid as an RNA product. In some embodiments, the RNA expression product is translated to a polypeptide or protein.

As used herein “integration vector” refers to a vector that is used to integrate a nucleotide sequence of interest into the genome of a target cell. Exemplary methods of integrating a nucleic acid into the genome of a cell are known in the art, e.g., CRISPR Cas9-based homologous recombination, retroviral or lentiviral transduction.

### Exemplary Embodiments

Disclosed herein are systems, kits, methods, and compositions useful for the functional screening of libraries of secreted proteins. In some embodiments, the systems, kits, methods, and/or compositions comprise one or more engineered cells expressing one or more test polypeptides and capable of conditionally expressing one or more reporter molecules. The embodiments described below are exemplary only and are not intended to be limiting.

1 An assay for protein or peptide discovery where
a. A library of cells is generated, each encoding for the secreted protein or peptide variant
b. Each cell is sequestered into a compartment
c. The functional activity is analyzed of each encoded variant peptide or protein in the library based on an analysis of the cell secreting the protein or peptide variant.
   2. The above, where >1,000 cells are screened at the same time.
   3. The above, where >10,000 cells are screened at the same time.
   4. Any of the above, where analysis of the protein or peptide secreting cell includes the detection of fluorescent protein expression, including but not limited to GFP, YFP, mCherry, and other fluorescent proteins.
   5. Any of the above, where analysis of the protein or peptide secreting cell includes the detection of enzyme expression, including but not limited to luciferase and horseradish peroxidase.
   6. Any of the above, where analysis of the protein or peptide secreting cell includes the detection of nucleic acids encoding for that protein.
   7. Any of the above, where analysis of the protein or peptide secreting cell includes the detection of nucleic acid sequences associated with the protein or peptide secreting cell.
   8. Any of the above, where nucleic acid sequences or barcodes indicate a secreted protein or peptide variant.
   9. Any of the above, where nucleic acid sequences or barcodes indicate a virus or pseudovirus infection event.
   10. Any of the above, where the protein function detected is virus neutralization.
   11. Any of the above, where the compartments comprise emulsion droplets, printed microwells, nanopens, 96-well plates, or 384-well plates.
   12. Any of the above, where the functional activity is the ability to block viral infection.
   13. Any of the above, where the functional activity is based on receptor activation in the same cell secreting the protein.
   14. Any of the above, where the functional activity is based on receptor deactivation in the same cell secreting the protein.
   15. The generation of a cell library capable of both protein secretion and functional assay of viral infection.
   16. The generation of a cell library capable of both protein secretion and functional analysis of engineered receptor activity.
   17. Any of the above, where the secreted protein is an antibody, Fab, IgG, IgM, IgA, ScFv, Fab′2, Fab2′, VHH, or other antibody or immunoglobulin expression format.
   18. Any of the above, where the sequence of the secreted protein can be tracked using a DNA barcode.
   19. Any of the above, where a virus or pseudovirus infection event can be tracked using a DNA barcode.
   20. Any of the above, where the readout for a virus neutralization assay is the insertion of DNA into cells by the virus, pseudovirus, virus-like particle, or recombinant viral particle.
   21. Any of the above, where the inserted DNA comprises a DNA barcode.
   22. Any of the above, where the readout for a virus neutralization assay is the expression of a fluorescent marker by the virus, pseudovirus, virus-like particle, or recombinant viral particle.
   23. Any of the above, where the readout for a virus neutralization assay is the expression of a surface protein reporter by the virus, pseudovirus, virus-like particle, or recombinant viral particle.
   24. Any of the above, where the readout for a virus neutralization assay is the expression of a growth selective marker by the virus, pseudovirus, virus-like particle, or recombinant viral particle.
   25. Any of the above, where the readout for engineered receptor agonism or antagonism is the expression of a fluorescent marker by the virus, pseudovirus, virus-like particle, or recombinant viral particle.
   26. Any of the above, where the readout for engineered receptor agonism or antagonism is the expression of a surface protein reporter by the virus, pseudovirus, virus-like particle, or recombinant viral particle.
   27. Any of the above, where the readout for engineered receptor agonism or antagonism is the expression of a growth selective marker by the virus, pseudovirus, virus-like particle, or recombinant viral particle.
   28. Any of the above, where the cell is a mammalian cell.
   29. Any of the above, where the cell is an insect cell.
   30. Any of the above, where the cell is a plant cell.
   31. Any of the above, where the cell is a bacterial cell.
   32. A screening method comprising: (a) detecting the presence and/or level of expression of a reporter molecule in a single, isolated, genetically engineered cell, wherein the cell presents a cell surface protein; and wherein the cell is engineered to: (i) secrete a heterologous test polypeptide; and (ii) express a reporter molecule if the test polypeptide activates the cell surface protein.
   33. A screening method comprising: (a) detecting the presence and/or level of expression of a reporter molecule in a single, isolated, genetically engineered cell, wherein the cell presents a cell surface protein; and wherein the cell is engineered to: (i) secrete a heterologous test polypeptide; and (ii) express a reporter molecule if the test polypeptide does not activate the cell surface protein.
   34. A screening method comprising: (a) contacting a single, isolated, genetically engineered cell with a test reagent, wherein the cell presents a cell surface protein, and wherein the cell is engineered to: (i) secrete a heterologous test polypeptide; and (ii) express a reporter molecule if one of the test polypeptide or the test reagent activates the cell surface protein; (b) detecting the presence and/or level of expression of the reporter molecule.
   35. A screening method comprising: (a) contacting a single, isolated, genetically engineered cell with a test reagent comprising a reporter molecule, herein the cell presents a cell surface protein; wherein the test reagent is capable of binding the cell surface protein presented by the cell, forming a reagent-protein complex, and wherein the test reagent gains entry into the cell when the reagent-receptor complex is formed; wherein the cell is engineered to: (i) secrete a heterologous test polypeptide; (b) detecting the presence and/or level of expression of the reporter molecule in the cells.
   36. The method of any of embodiments 32-35, wherein the cell comprises a mammalian cell, an insect cell, an avian cell, a yeast cell, a plant cell, or a bacterial cell.
   37. The method of any of any of the previous embodiments, wherein the cell surface protein comprises an endogenous receptor.
   38. The method of any of the previous embodiments, wherein the cell is engineered to express the cell surface protein.
   39. The method of embodiment 38, wherein the cell surface protein comprises a heterologous receptor.
   40. The method of any of any of the previous embodiments, wherein secretion of the test polypeptide is constitutive.
   41. The method of any of embodiments 32-39, wherein secretion of the test polypeptide is inducible.
   42. The method of any of the previous embodiments, wherein the single, isolated, genetically engineered cell is in a well of a multi-well plate.
   43. The method of any one of embodiments 32-42, wherein the single, isolated, genetically engineered cell is in a chamber of a microchip.
   44. The method of any one of embodiments 32-42, wherein the single, isolated, genetically engineered cell is in a microfluid droplet, such as an emulsion droplet.
   45. The method of any one of embodiments 32-42, wherein the single, isolated, genetically engineered cell is in a Nanopen™.
   46. The method of any one of the previous embodiments, wherein the reporter molecule comprises a fluorescent marker, an enzyme, a tagged protein, or a nucleic acid sequence.
   47. The method of any one of the previous embodiments, wherein the cell comprises a human cell.
   48. The method of any one of the previous embodiments wherein the reporter molecule comprises a nucleic acid sequence, optionally a barcode sequence, and detecting the presence and/or level of expression of the reporter molecule comprises one or more of an amplification reaction and a sequencing reaction, optionally a single cell sequencing reaction.
   49. The method of any one of embodiments 32-48, wherein the reporter molecule comprises a fluorescent moiety, and detecting the presence and/or level of expression of the reporter molecule comprises fluorescence activated cell sorting.
   50. The method of any one of the previous embodiments, wherein the method further comprises sequencing the nucleic acids encoding the heterologous test polypeptide.
   51. The method of any one of embodiments 1-3, or 5-19, wherein heterologous test peptide comprises a variant of the receptor ligand.
   52. The method of embodiment 51, wherein the variant is derived from a library of ligand variants.
   53. The method of any one of embodiments 3, or 5-19, wherein the test polypeptide comprises a variant of a cell surface protein ligand, and wherein the test reagent comprises an agonist or an antagonist of receptor activation by the wild-type ligand.
   54. The method of any one of embodiments 3 or 5-19, wherein the test reagent comprises a cell surface protein ligand, and wherein the test polypeptide is derived from a library of potential agonists or antagonists of receptor activation by the ligand.
   55. The method of any one of embodiments 1-19, wherein the test polypeptide comprises an antibody, antibody-derived format, a nanobody, VHH, or antigen binding fragment thereof.
   56. The method of embodiment 24, wherein the antibody or antibody-derived format, a nanobody, VHH, or antigen binding fragment is derived from a library of antibodies, or antigen binding fragments.
   57. The method of any of embodiments 4, 5-19, or 24-25, wherein the test reagent comprises one or more of a virus, virus-like particle, pseudoviruses, and recombinant viral particle, and wherein the cell surface protein comprises a component of viral entry into the cell.
   58. The method of embodiment 26, wherein the virus is selected from Coronavirus A, B, C, or D, Flavivirus, Lentivirus, Influenza A, B, or C.
   59. The method of embodiment 26, wherein the virus selected from HIV, SARS-CoV-2, Epstein-Barr virus, herpes simplex virus, cytomegalovirus, respiratory syncytial virus, Ebola virus, Marburg virus, Dengue virus, and Yellow Fever Virus.
   60. The method of embodiment 26, wherein the virus comprises a SARS-CoV-2 virus, and wherein the cell surface protein comprises a human angiotensin-converting enzyme 2 (hACE2).
   61. The method of embodiment 29, wherein the cell is engineered to express Transmembrane Serine Protease 2 (TMPRSS2).
   62. A composition, kit, or system, comprising the genetically engineered cell of any of the previous embodiments.
   63. A kit comprising: (a) a vector encoding a heterologous test polypeptide; (b) a vector encoding a reporter molecule, expression of which is activated if the heterologous test polypeptide activates a cell surface protein, optionally, wherein one or more of the vectors are expression vectors, or, optionally, wherein one or more of the vectors are integration vectors.
   64. A kit comprising: a) a vector encoding a heterologous test polypeptide; (b) vector encoding a reporter molecule, expression of which is activated if the heterologous test polypeptide does not activate a cell surface protein, optionally, wherein one or more of the vectors are expression vectors, or, optionally, wherein one or more of the vectors are integration vectors.
   65. A kit comprising: (1) a test reagent and (2) (a) a vector encoding a heterologous test polypeptide; (b) a vector encoding a reporter molecule, expression of which is activated if either the heterologous test polypeptide or test reagent activates a cell surface protein, optionally, wherein one or more of the vectors are expression vectors, or, optionally, wherein one or more of the vectors are integration vectors.
   66. A kit comprising: (1) a test reagent comprising a reporter molecule and (2) (a) a vector encoding a heterologous test polypeptide, optionally, wherein one or more of the vectors are expression vectors, or, optionally, wherein one or more of the vectors are integration vectors.
   67. The kit of any of any one of embodiments 36-35, wherein the one or more nucleic acids further encode (c) a cell surface protein.
   68. The kit of any one of embodiments 63-66, wherein the heterologous test polypeptide is operably linked to a promoter.
   69. The kit of embodiment 68, wherein the promoter is a constitutive promoter.
   70. The kit of embodiment 68, wherein the promoter is an inducible promoter.
   71. The kit of any of embodiments 63-70, wherein the reporter molecule comprises a fluorescent marker, an enzyme, a tagged protein, or a nucleic acid sequence.
   72. The kit of any of embodiments 66-71, wherein the test reagent comprises one or more of a virus, virus-like particle, pseudoviruses, and recombinant viral particle.
   73. The kit of embodiment 72, wherein the virus is selected from a Coronavirus A, B, C, or D, Flavivirus, Lentivirus, and Influenza A, B, or C.
   74. The kit of embodiment 72, wherein the virus is selected from HIV, SARS-CoV-2, Epstein-Barr virus, herpes simplex virus, cytomegalovirus, respiratory syncytial virus, Ebola virus, Marburg virus, Dengue virus, and Yellow Fever Virus.
   75. The kit of embodiment 72, wherein the pseudovirus comprises a peptide, polypeptide, or protein derived from a Coronavirus A, B, C, or D, Flavivirus, Lentivirus, Herpesvirus, or Influenza A, B, or C.
   76. The kit of embodiments 72, wherein the pseudovirus comprises a peptide, polypeptide, or protein derived from HIV, SARS-CoV-2, Epstein-Barr virus, herpes simplex virus, cytomegalovirus, respiratory syncytial virus, Ebola virus, Marburg virus, Dengue virus, or Yellow Fever Virus.
   77. The kit of any of embodiments 63-65 or 68-71, wherein the heterologous test peptide comprises an antibody, or a portion thereof.
   78. The kit of embodiment 77, wherein the heterologous test peptide is a single chain variable fragment (scFv) or a nanobody.
   79. A kit comprising: (1) a vector for expressing a heterologous test polypeptide, (2) a genetically engineered cell comprising: (a) a nucleic acid encoding a reporter, expression of which is activated if the heterologous test polypeptide activates a cell surface protein.
   80. A kit comprising: (1) a vector for expressing a heterologous test polypeptide, (2) a genetically engineered cell comprising: (a) a nucleic acid encoding a reporter, expression of which is activated if the heterologous test polypeptide does not activate a cell surface protein.
   81. A kit comprising: (1) a vector for expressing a heterologous test polypeptide, (2) a genetically engineered cell comprising: (a) a nucleic acid encoding a reporter, expression of which is activated if the heterologous test polypeptide activates a cell surface protein, and optionally, (3) a test reagent.
   82. A kit comprising: (1) a vector for expressing a heterologous test polypeptide, (2) a genetically engineered cell; (3) a test reagent comprising a reporter molecule, wherein the test reagent is capable of binding a cell surface protein presented by the cell, forming a reagent-surface protein complex, and wherein the test reagent gains entry into the cell when the reagent-protein complex is formed.
   83. The kit of any of embodiments 79-82, wherein the genetically engineered cell further comprises: (c) a nucleic acid encoding a heterologous cell surface protein.

### EXAMPLES

The following Examples are illustrative and should not be interpreted to limit the scope of the claimed subject matter.

### Example 1: Establishment of a Cell Line for Concurrent mAb Secretion and Viral Infection

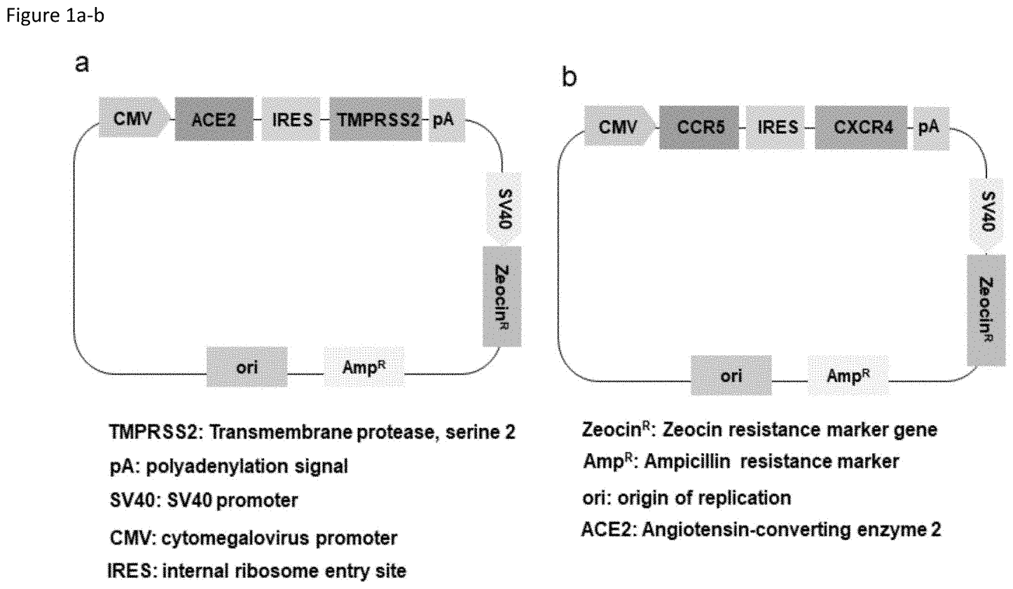

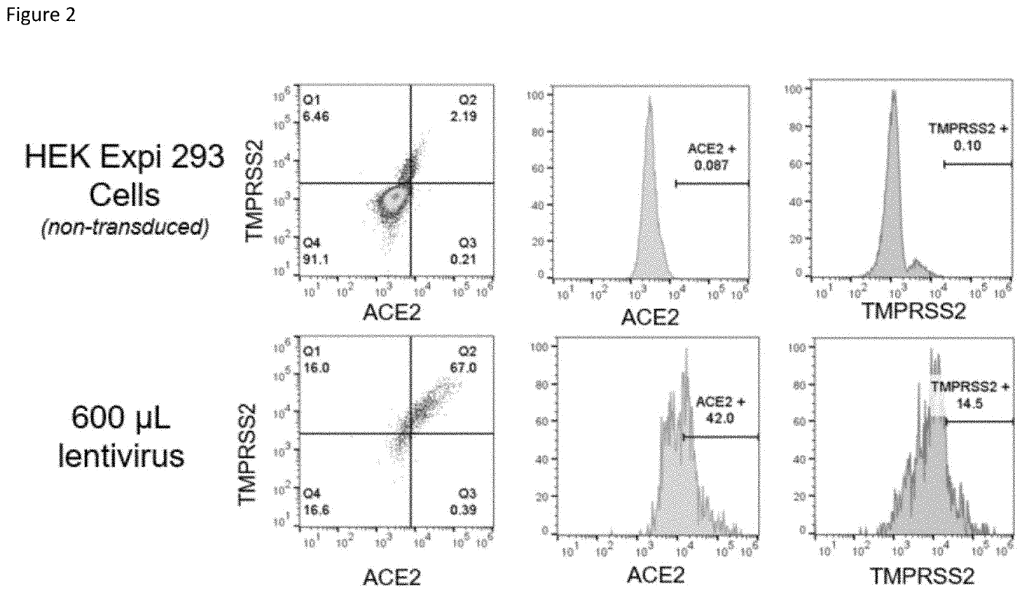

In this working example, SARS-CoV-2 receptor/co-receptors and anti-SARS-CoV-2 antibody were used as an example application of a neutralization assay performed with the same cell line for both protein secretion and viral infection concurrently. Aa mammalian cell line was developed, expressing anti-viral antibodies and their respective viral entry receptors or co-receptors to permit viral infection concurrently with antibody secretion. As an example, virus application, an anti-SARS-CoV-2 antibody and its receptor human Angiotensin-converting enzyme 2 (hACE2) and/or Transmembrane Serine Protease 2 (TMPRSS2) were expressed in a mammalian cell line (FIG. 1). A bicistronic vector was constructed containing a human cytomegalovirus promoter, the ACE2 surface receptor of SARS-CoV-2 infection, an internal ribosome entry site (IRES), and the TMPRSS2 gene, allowing co-expressing hACE2 and TMPRSS2 in a mammalian expression vector. (The TMPRSS2 gene is optional and is not required for SARS-CoV-2 infection but can enhance the ability of virus to (8) infect some cells). This expression cassette was cloned into a vector with a selectable marker for plasmid transfection, enabling transformed cell selection using the selectable marker. We transfected this plasmid HEK293 cells by mixing the plasmid with lipofectamine transfection reagents. Two days after, the cell culture media was replenished, and the selection reagent respective to the selectable marker was added to the culture media for initiating the selection process. After 7-14 days, we obtained a stable cell pool, which was resistant to the selectable reagent. Then this pool of cells was stained with both fluorescent-conjugated anti-ACE2 and fluorescent-conjugated anti-TMPRSS2 (FIG. 2). Cells were sorted for ACE2 and TMPRSS2 expression and the cells were rested for recovery. After the cell recovery with regular growth, limiting dilution cloning was performed to isolate a single clone. After 10-15 days, the single clone formed a cell colony which was transferred into 24 well plates allowing the cells to expand. After the cell expansion, we stained the clones with antiACE2 and anti-TMPRSS2 and selected the clone with highest ACE2 and TMPRSS2 expression (Named, HEKACE2/TMPRSS2).

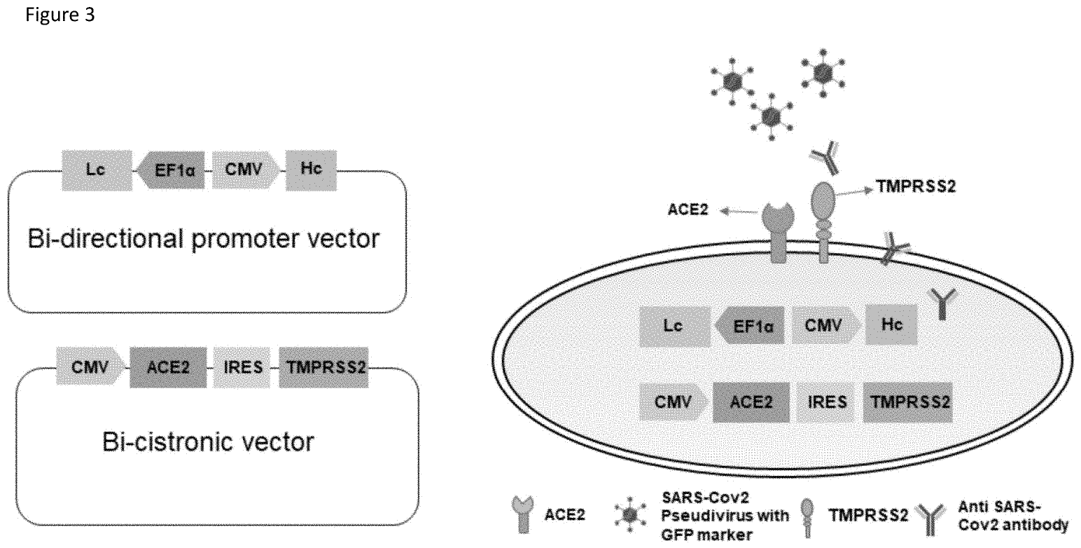

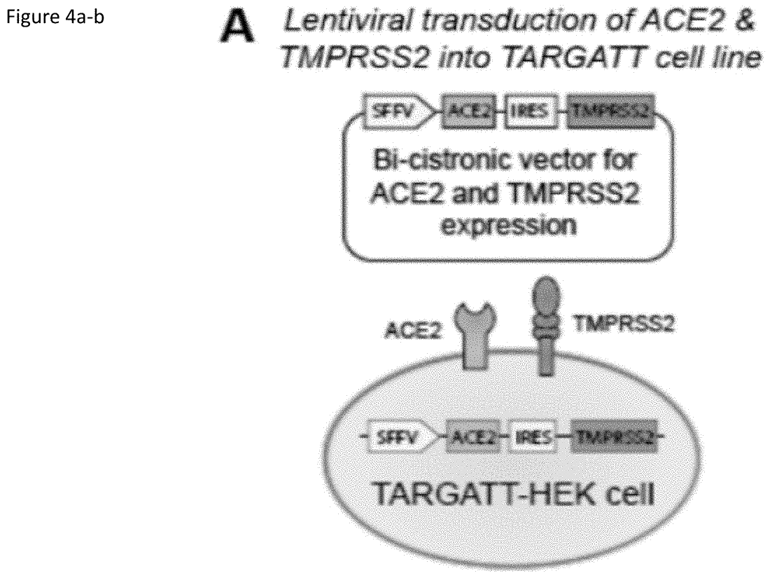

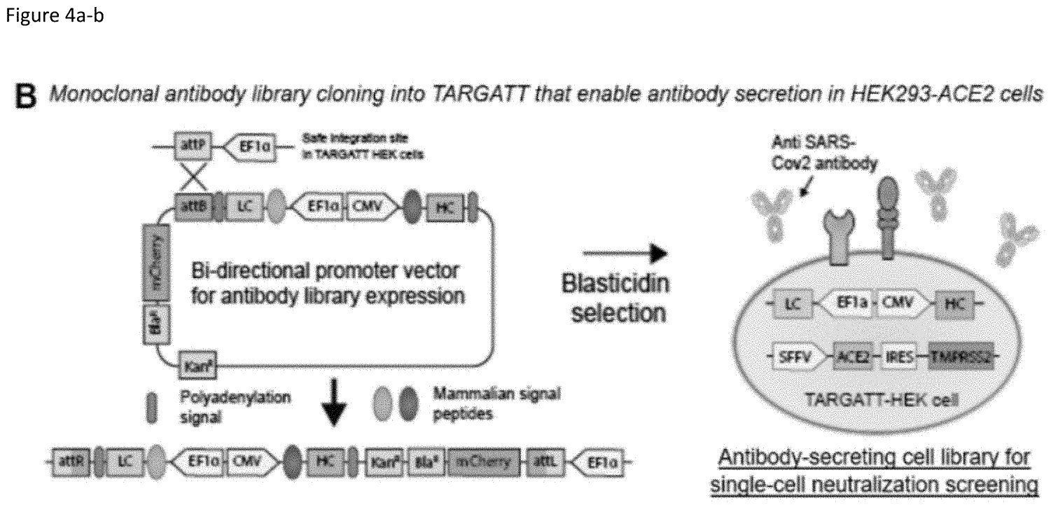

We then transformed these cells to express complete IgG of antibodies with known SARS-CoV-2 neutralization capacity. Alternatively, we could express other IgG fragments such as single-chain variable fragment (Scfv), antigen-binding fragment (Fab), or bi-specific antibody. We cloned the desired antibody or antibody fragment into a mammalian vector with a selectable marker. Followed by transfection and selection with selection marker reagent, the IgG expressing HEKACE2/TMPRSS2 stable pool was then subjected to limiting dilution cloning to isolate the individual secreted protein expressing clone, which in this case was an antibody IgG. After 10-15 days of cell expansion, we transferred 50 uL of cell culture media to evaluate the IgG expression by direct ELISA. The ELISA was performed by coating the IgG overnight in 96 well plates. The plates were washed with Phosphate-buffered saline with 0.05% Tween 20 (PBST) and were blocked with 5% BSA in PBST for 2 hours. We washed the plates three times with PBST and added the HRP-conjugated rabbit anti-human Fc antibodies onto the well and incubated for two hours. The plates were washed with PBST four times and the 3,3′,5,5′-Tetramethylbenzidine Liquid Substrate was added for HRP reaction and stopped with 2M H2504 for detection. We analyzed the plates using a plate reader at the absorbance wavelength of 450 nm. We then compared the absorbance of the IgG expressed by HEKACE2/TMPRSS2 stable clones with IgG standard to estimate the relative IgG expression yield. We selected the highest IgG expressing clone as our candidate clone. We generated a cell line include ACE2, TMPRSS2 and IgG allowing neutralization assay to be performed in a single cell (FIG. 3). Several strategies for cell line development can be used (FIG. 4).

### Example 2: Quantification of Infection Against Viruses or Pseudoviruses in a Modified Mammalian Cell Line Capable of Soluble Protein Secretion

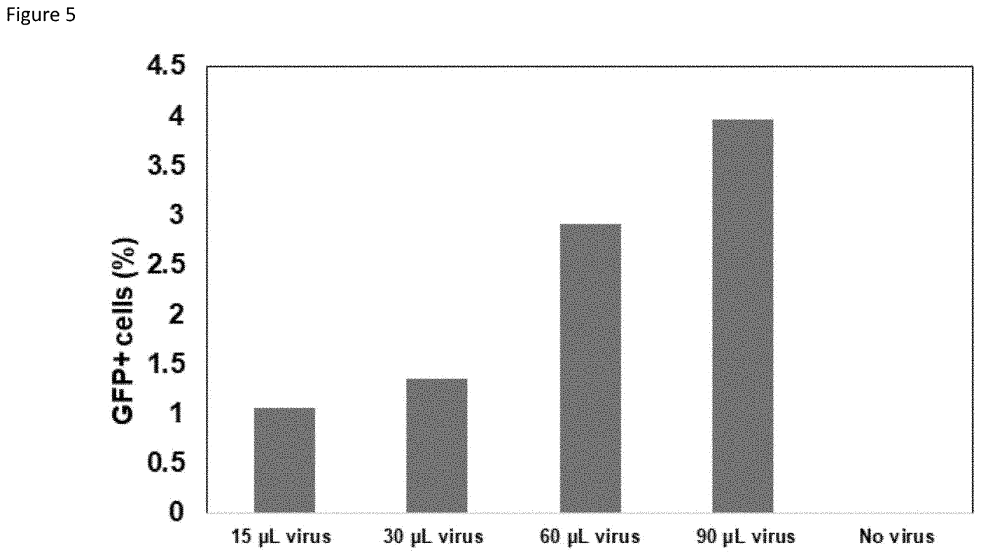

The HEK293 cell line can be used for protein expression or secretion in lab experiments. In this working example, we used a HEK293 cell line with ACE2 expression but no TMPRSS2 expression for the pseudovirus neutralization assay (Named HEKACE2). We infected a HEKACE2 with a strain of lentiviral-based pseudovirus encoding SARS-CoV-2CoV2 spike protein on the viral surface with a GFP reporter gene in the viral expression vector; SARS-CoV-2 pseudovirus infected cells would thus express GFP. We detached the HEKACE2 cells with 0.05% trypsin and stopped the trypsin reaction with DMEM media with 10% FBS. We then counted the cell density and resuspended cells to a density of 3×10⁵ cell/mL and added 100 μL of the cell suspension to each well in the 96 well plate. We retrieved an aliquot of frozen pseudovirus and added various amount of the virus (15 μL, 30 μL, 60 μL and 90 μL) to the 96 wells. The 96-well plates were incubated at 37° C. for 48-72 hours before neutralization was quantified by acquiring GFP signal using flow cytometry. As indicated in FIG. 5, the percentage of the pseudovirus infected HEKACE2 cells, reflected by the percentage GFP positive cells, correlated to the amount of virus added (FIG. 5).

### Example 3: Enabling Protein Secretion in Single Cells, with a Library of Encoded Protein Variants

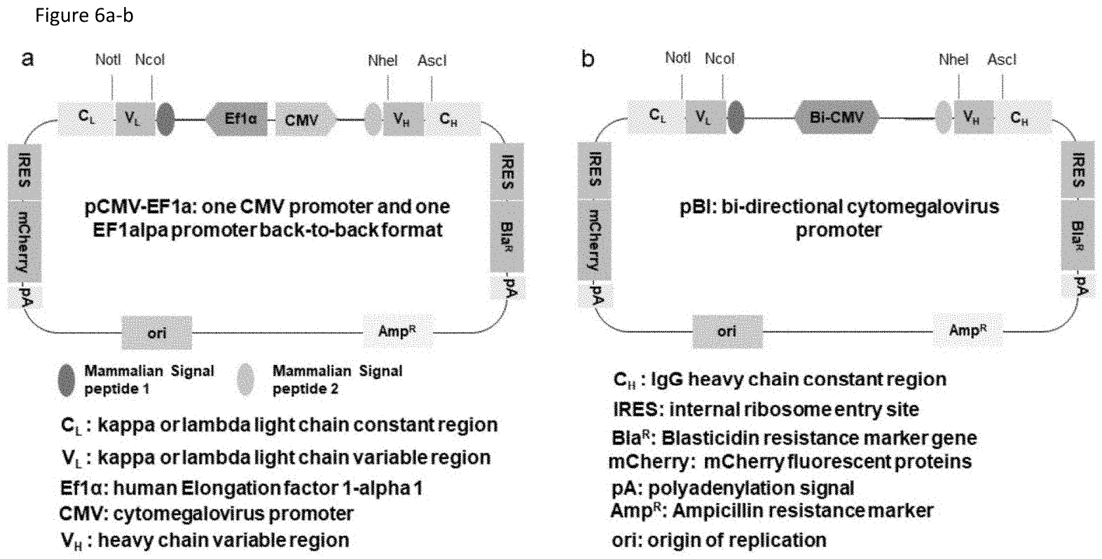

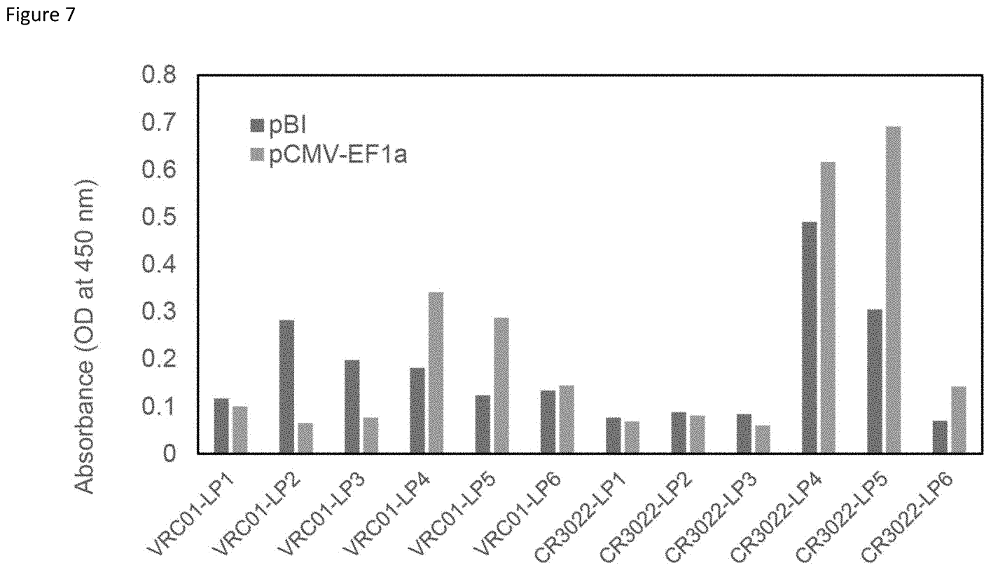

In this working example, we show methods to enable protein secretion in single cells for subsequent protein secretion and assay-based selection. In this example, the secreted protein is an antibody IgG. We can obtain libraries of natively paired antibody heavy and light chain variable regions (VH:VL) from patients SARS-CoV-2 infection as described in the protocol in McDaniel et al⁽³⁾. We cloned paired VH:VL sequences into a plasmid vector with one CMV promoter and one EF1alpa promoter (pCMV-EF1a, FIG. 6) or a vector with a bi-directional promoter (FIG. 6, pBI), similar to a format previously described_{(10,11,12,13,14)}. The cloning of a VH:VL library into the pCMV-EF1a and pBI utilize NotI and NheI cutting sites to clone the amplicon into the backbone vectors without the promoter and we then cloned in the dual promoters (CMV and EF1a) or the bi-directional vector (Bi-CMV) using NheI and NcoI site on the leader peptide region of the heavy chain and light chain respectively. Prior studies have shown that changing a protein's leader peptide can modulate the level of protein expression^{10,11,12,13,14)}. We designed different leader peptides to achieve varied levels of protein secretion. We designed three leader peptides that included an NheI cut site for the heavy chain (Lel1, Alb2 and L2B) and two leader peptides that included a NcoI cut site for the light chain (Lel2 and Alb1) (Table 1). We expressed two antibodies (anti-HIV antibody VRC01 and anti-SARS-CoV antibody, CR3022) with six different heavy chain and light chain leader peptide combinations (Table 2) in two different vectors (pBI or pCMV-EF1a). We transfected these 24 IgG constructs into HEKACE2 cells (without TMPRSS2 expression) using lipofectamine. Three days post transfection, we detected the IgG expression via ELISA as described in Example 1. As shown in FIG. 6, for both VRC01 and CR3022 antibodies, we observed that all leader peptide combinations enable IgG expression, and different leader peptide combinations could be selected for modulation of the concentrations of secreted antibody desired. With LP4 and LP5 we observed the highest two IgG expressions in the pCMV-EF1a vector (FIG. 7). We generated a stable IgG expressing pool by adding Blasticidin to a final concentration of 5 μg/mL.

TABLE 1
Leader peptide amino acid sequences and DNA sequences

| Light Chain | Light chain leader peptide | |
|---|---|---|
| leader peptide | amino acid sequence | DNA sequence |
| Lel1 | MSKRGASNEMALLLLLL | ATGAGCAAGAGGGGCGCCAGCAACGAGATGGCCC |
| | GLLQQAMA (SEQ ID NO: | TGCTGCTGCTGCTGCTGGGCCTGCTGCAGCAGgccatg |
| | 17) | gcg (SEQ ID NO: 18) |
| Alb2 | MKWVTFISILFLLSSAM | ATGAAGTGGGTGACCTTCATCAGCCTGCTGTTCC |
| | A (SEQ ID NO: 19) | TGTTCAGCAGCgccatggcg (SEQ ID NO: 20) |
| L2B | MKYLIPTAAAGLLLLAA | ATGAAGTATTTGTTGCCGACGGCGGCGGCGG |
| | QPAMA (SEQ ID NO: 33) | GGTTGTTGTTGTTGGCGGCGCAGCCGgccatggcg |
| | | (SEQ ID NO. 34) |
| Heavy chain | Heavy Chain leader peptide | |
| leader peptide | amino acid sequence | DNA sequence |
| Lel2 | MSKRGASNEMALLLLLL | ATGAGCAAGAGGGGCGCCAGCAACGAGATGGCCC |
| | GLLASVLA (SEQ ID NO: | TGCTGCTGCTGCTGCTGGGCCTGCTGgctacgttttagca |
| | 21) | (SEQ ID NO: 22) |
| Alb1 | MKWVTFISLLFLFASVLA | ATGAAGTGGGTGACCTTCATCTCCCTGCTTGC |
| | (SEQ ID NO: 23) | CTGTTCGCTAGCGTTTTAGCA (SEQ ID NO: 24) |

TABLE 2
Heavy chain and light chain leader peptide combinations

| Leader | Heavy chain | Light chain |
|---|---|---|
| peptide | leader | leader |
| pair name | peptide | peptide |
| LP1 | Alb1 | L2B |
| LP2 | Alb1 | Le11 |
| LP3 | Alb1 | Alb2 |
| LP4 | Le12 | Le11 |
| LP5 | Le12 | Alb2 |
| LP6 | Le12 | L2B |

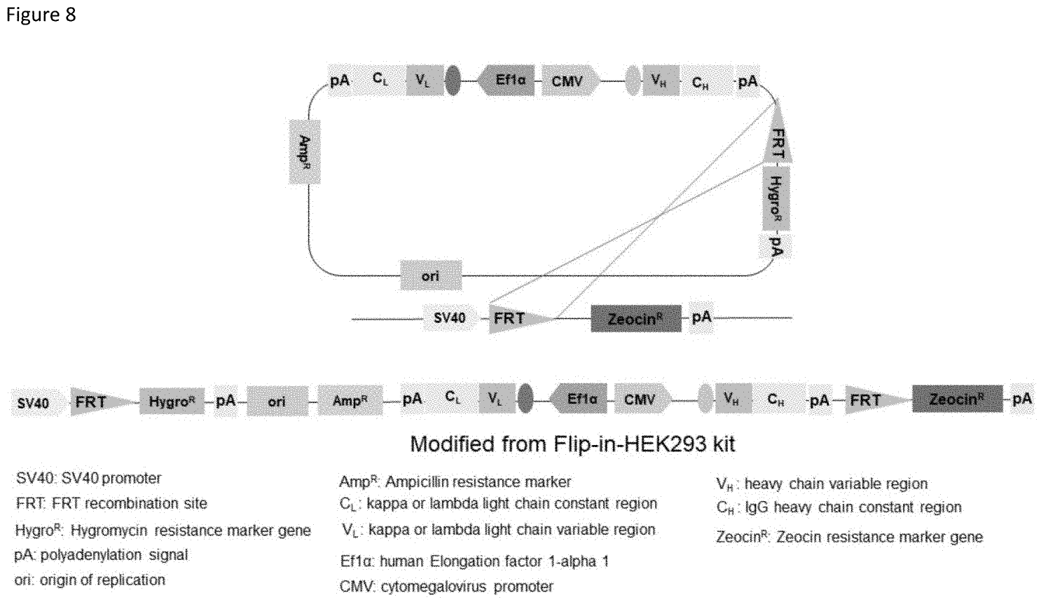

Alternatively, we could express the natively paired VH:VL into HEKACE2 derived from the Flip-In HEK293 kit via Flp recombinase-mediated integration at the FRT site (FIG. 8). To do so, we would first clone the IgG expression gene cassette into the pcDNA5/FRT vector. We would then co-transfect the engineered pcDNA5/FRT vector with Flp recombinase vector pOG44 into the HEK-Flp-In 293 with ACE2 expression. We would generate a library of IgG expressing cells by hygromycin selection. The Flp-mediated cloning has an advantage in that only a single protein variant is encoded by each cell, which is helpful for the selectivity of our assay, although not strictly necessary for assay implementation.

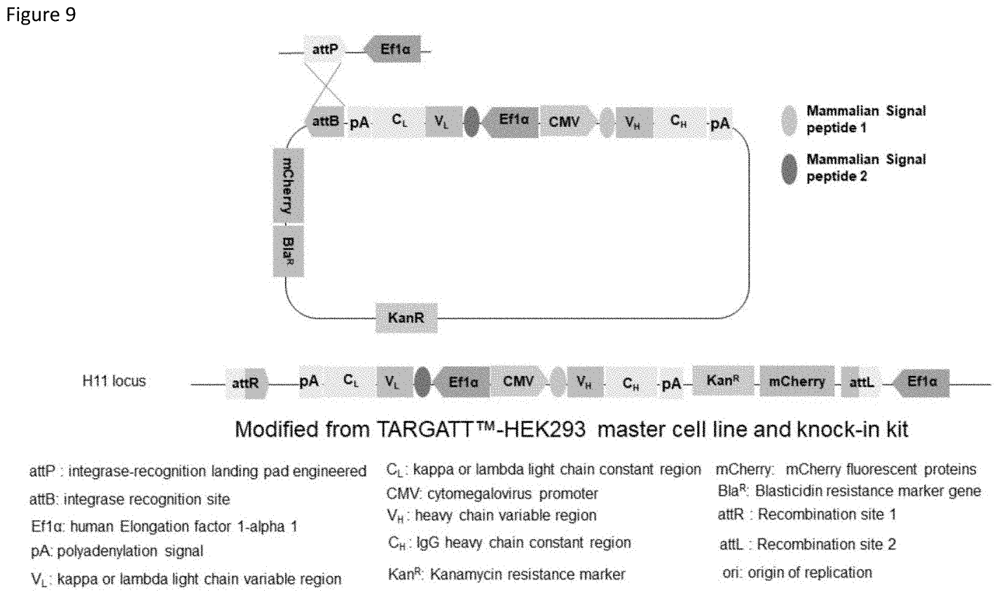

In an alternative working example, we used an integrase-based gene integration system to express IgG from HEK293ACE cells derived from the TARGATT™-HEK293 master cell lines. We cloned the IgG expression gene cassette into a donor vector containing the integrases recognition site, attB, blasticidin resistance marker and mCherry (FIG. 9). We then co-transfected the donor plasmid and integrase expression plasmid into an engineered HEK cell line stably expressing ACE2. The attP landing pad was at the hH11 gene locus

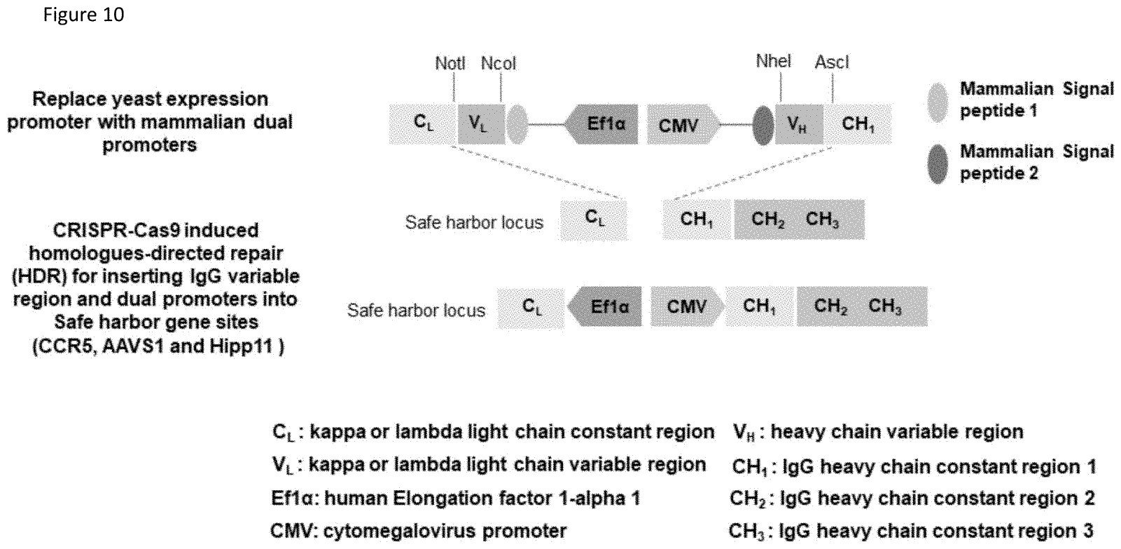

In another working example, we used the CRISPR homologous directed repair platform system to express the IgG in HEK-ACE2 cells. We co-transfected donor IgG expressing cassette (VH:VL sequences with dual promoter or bi-directional promoter) with homologous arms (FIG. 10). The gRNA/Cas9 expressing vector provided integration of the natively paired VH:VL sequence into a safe harbor gene locus. Targeted safe harbor loci include CCR5, AAVS1, and Hipp11 (FIG. 10).

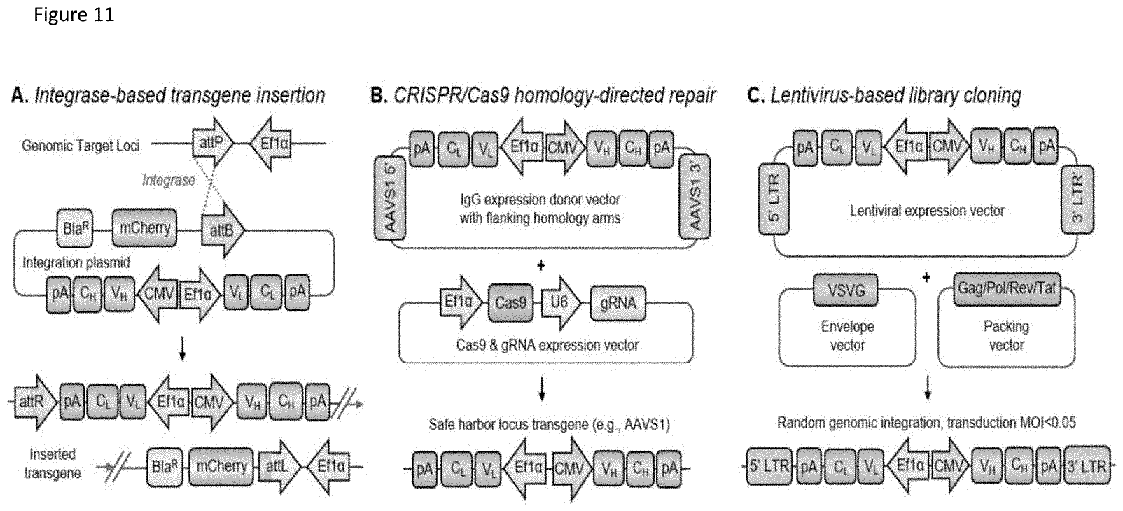

The cloning and transformation methods can be suitably matched to the cell lines and cell-based functional activity model of interest. Several different cloning and transformation methods can be suitably used for generating libraries of secreted proteins into mammalian or other cells (FIG. 11). Other types of cloning can be used to insert nucleic acids for protein secretion into host cells, which can include, without limitation, lentiviral gene transfer, infectious molecular clones, adenoviral vectors, adeno-associated viral vectors, chemical DNA transfection, chemical RNA transfection, mRNA encapsulated by nanoparticles, DNA encapsulated by nanoparticles, or other methods known to the art to induce cell expression of desired proteins and plasmid vectors.

### Example 4: The Generation of Antibody Protein Libraries for Cloning and Soluble Antibody Functional Analysis in VH:VL Bidirectional Format

In this prophetic example, randomly paired VH:VL libraries or mutational VH:VL libraries can be synthesized via a gene synthesis service, where the VH and VL genes of the antibody are linked by a DNA linker. Alternatively, VH:VL gene libraries can be amplified directly from human, mouse, or non-human primate samples, as reported previously⁽¹⁵⁾. In some embodiments, we can introduce diversity into the libraries using error-prone PCR, site-saturation mutagenesis, and/or DNA shuffling. We can clone the VH library by using a combination of NotI and NcoI and clone the VL library by using a combination of NheI and AscI in a dual promoter (back-to-back format), or we can maintain the bi-directional promoter format as illustrated in FIG. 6, by using first NotI and AscI to clone the full construct, and then using NcoI and NheI to clone in the bidirectional promoter, similar to previous reports_{(16,17)}. We can transfect this plasmid into the HEKACE2 cells for IgG expression. Alternatively, we can clone the gene cassette (dual promoters or bi-directional promoter with a heavy chain and a light chain) into the FLP/FRT based gene integration donor plasmids (FIG. 8), integrase-based donor plasmids (FIG. 9), or CRISPR/Cas9 donor plasmid for stable IgG integration (FIG. 10) into a safe harbor gene locus. Several possible cloning strategies can be used for bidirectional antibody expression (FIG. 11).

### Example 5: Methods to Enable Synthetically Generated Antibody Expression in Standard One-Direction Format

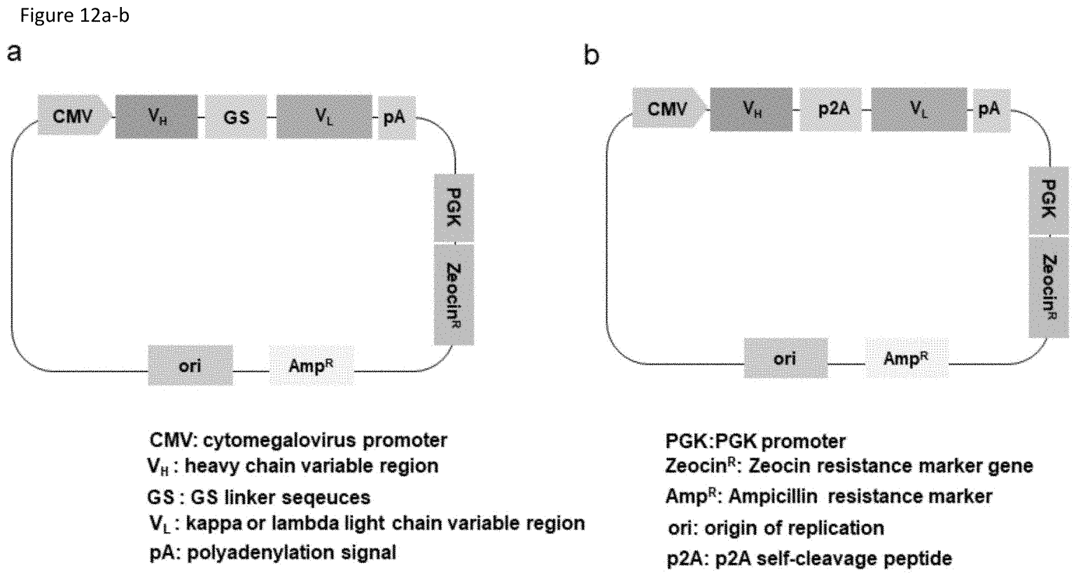

We can synthesize randomly paired VH:VL libraries or mutational VH:VL library via gene synthesis service. We can then clone the VH and VL separately into a mammalian expression vector and express the IgG in one open reading frame as illustrated in FIG. 12a.

IgG can be expressed in a single-chain variable fragment format with GS linker in between the heavy chain and light chain variable region.

Alternatively, full IgG can be expressed in a bi-cistronic format with a p2A cleavage peptide between the IgG heavy chain and the light chain (FIG. 12B) (see Yellow Fever working examples, below). We transfected the plasmid and expressed the IgG.

We can integrate the one-directional format of IgG into a safe harbor locus expression site. Options for integration include FLP/FRT based gene integration (FIG. 8), integrase-based donor plasmid (FIG. 9), transposon-based integration, or with the use of CRISPR/Cas9 donor plasmids for stable IgG integration (FIG. 10). Several cloning and transformation methods are suitable, depending on the cell line and functional model to be used for the secreted proteins (e.g., FIG. 11).

### Example 6: Application of the Secreted Protein Assay in Well Plates as a Compartment for Selection of Antibodies with Viral Neutralization Properties

In this working example, we show a functional assay using secreted protein along with a readout of secreted protein activity in the same cell lines. In this example, the secreted protein is an antibody, and the activity to be assayed is neutralization of SARS-CoV-2 pseudovirus, and the selection marker is GFP.

2×10⁴ of HEKACE2 cells were seeded in a 96 well plate to reach a confluency of 70% at the time of transfection. Following the protocol included with the Lipofectamine™ 3000 Reagent Kit (Invitrogen), 100 ng of mAb in mammalian expression vector pBI per well was diluted in 5 μL of Opti-MEM™ Reduced Serum Media (Thermo Fisher Scientific), and 0.2 μL of P3000™ Reagent was added. Separately, 0.2 μL of Lipofectamine™ 3000 Reagent was also diluted in 5 μL Opti-MEM™. The diluted DNA was added to the diluted Lipofectamine™ 3000 Reagent and incubated at room temperature for 12 minutes. This DNA-lipid complex was then added to the HEKACE2 cells and incubated at 37° C. for 3 days. As the pBI vector contains mCherry on the light chain of the mAb, the cells could be visualized using a fluorescence microscope or flow cytometry following transfection to confirm gene expression. Three days following transfection, the neutralization activity of the antibodies was be measured as described below.

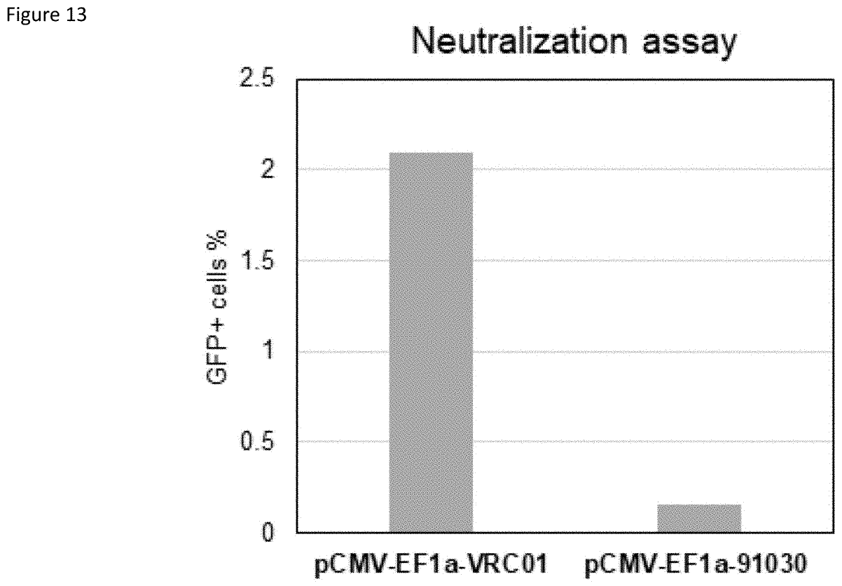

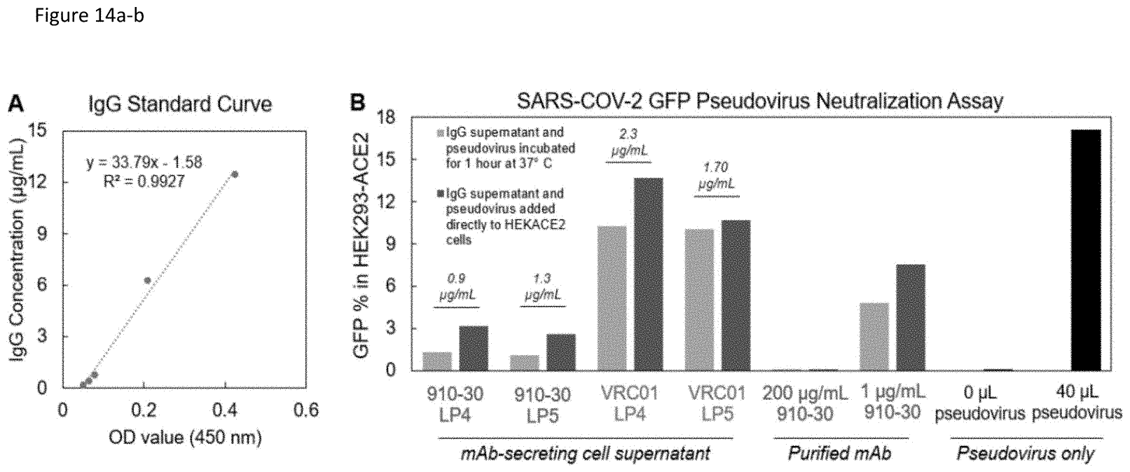

SARS-CoV-2 Wuhan Hu-1 GFP reporter virus particles (Integral Molecular) were thawed and placed on ice, 60 μL of the reporter virus particles were added directly to the cell media. The 96-well plates were incubated at 37° C. for 48-72 hours before neutralization was quantified by acquiring GFP signal using flow cytometry. ELISA analysis of IgG expression indicated that VCR01 has a higher IgG expression level than that of the antibody 910-30⁽¹⁷⁾, an anti-SARS-CoV-2 antibody. Neutralization assay showed that HEKACE2 expressing VCR01 exhibited a higher GFP population than the HEKACE2 cell expressing 910-30 (FIG. 13). Similar results were obtained when repeating the experiments (FIG. 14).

### Example 7: Selection of Neutralizing Antibodies from a Library of Antibodies Encoded by Cells Capable of Both Antibody Secretion and Pseudovirus Infection

In this prophetic example, we generate a mixture of the stable cell lines expressing secreted VRC01 and 910-30, as described in Example 6. We perform limiting dilution isolation to generate single cells in a 96-well plate, with an average of 0.25 cells per well. Cells are permitted to expand for 40 days after the limiting dilution cloning, and then we add 30 μL of pseudovirus for direct neutralization assay. We retrieve the cells and sort the GFP+ populations, which were enriched for cells that were not protected from infection by the antibodies they secrete (i.e., were expressing non-neutralizing antibodies). We also sort the GFP− population to enrich for cells protected from infection (i.e., expressing neutralizing antibodies.) We retrieve the paired antibody DNA gene sequences from GFP− and GFP+ populations by extracting the RNA and performing RT-PCR for the antibody genes⁽²²⁾. We perform high-throughput sequencing to obtain the VH:VL information in the GFP− and GFP+ populations. We compare the frequency of antibody variants in each population and determine the neutralization capacity of each antibody in the population⁽²¹⁾ pool. We find that VRC01 sequences were comparatively enriched in the GFP+ virus-infected group, whereas 910-30 sequences were comparatively enriched in the GFP− group, and these quantitative signals of selection demonstrate the ability of our secreted protein assays to test for the virus neutralization capacity of encoded antibodies secreted by cells.

To discover natively paired VH:VL antibodies directly from B cells, we clone the native VH:VL library from SARS-CoV-2 human patient samples into the IgG expressing vector and express in HEKACE2 cells as described in Example 3, Example 4 and Example 5. We perform limiting dilution cloning isolate the single cell in a 96 well with one cell per well. Forty days after the limiting dilution cloning, we add the 30 μL of pseudovirus for direct neutralization assay. We sort the GFP+ population, which was enriched for non-neutralizing antibodies. We sort the GFP-population to enrich the population for neutralizing antibodies. We retrieve the paired antibody DNA gene sequences from GFP- and GFP+ populations by extracting the RNA and performing RT-PCR for the antibody genes⁽²²⁾. We perform high-throughput sequencing analysis to obtain the VH:VL information. We compare the frequency of antibody variants in each population to determine the identify of neutralizing antibodies in the population⁽²¹⁾.

### Example 8: Application of Secreted Protein Functional Assays in Printed Microchambers as a Compartment

In this prophetic example, we first generate the paired VH:VL expressing HEKACE2 cells by one of the methods described in Example 3, Example 4, and Example 5. A population of cells is added into 125-pl wells molded in polydimethylsiloxane (PDMS) slides⁽²⁾. Each slide contains 1.7×10⁵ wells; we process four slides simultaneously to include 68,000 IgG expressing HEKACE2/TMPRSS2 cells at an approximately 1:10 cell-to-well ratio occupancy, enabling a greater than 95% probability of single-cell per well according to Poisson statistics. We incubate the slide at 37° C. 5% CO₂ incubator for overnight, allowing IgG secretion. SARS-CoV-2 pseudovirus is deposited over the microwells to diffuse inside and the PDMS slides are sealed with a dialysis membrane. We incubate the slides for 16 hours allowing the virus entry to the cells. The slides are washed, and the live cells are recovered from the slides in the presence of high concentrations (1 mg/mL) of soluble 910-30 neutralizing IgG to prevent subsequent viral infection once cells are pooled together. The cells are seeded into a 24 well plate to recovery and expand at 37° C. 5% CO₂ incubator for two days. We centrifuge the cell and resuspend in FACS buffer. We recover GFP- and GFP+ populations and extract the RNA and performing RT-PCR for the antibody genes⁽²²⁾. We perform high-throughput sequencing analysis to obtain the VH:VL information. We compare the frequency of antibody variants in each population to determine the identify of neutralizing antibodies in the population^{(3,21)} as described in Example 6.

Alternatively, we screen the anti-SARS-CoV-2 neutralizing antibody via Lightning Optofluidic System. We load the IgG expressing HEKACE2/TMPRSS2 cells onto the OptoSelect™ chip with NanoPen™ chamber to isolate them in a one-cell-per-chamber basis and incubate overnight for cells to secret antibody. The SARS-CoV-2 pseudovirus is added to the chambers and incubate for three days. The live cells are recovered from the Nanopens™ in the presence of high concentrations (1 mg/mL) of soluble 910-30 neutralizing IgG to prevent subsequent viral infection once cells are recovered together. We isolate GFP- and GFP+ populations using fluorescence activated cell sorting (FACS) and extract the RNA and perform RT-PCR for the antibody genes⁽²²⁾. We perform high-throughput sequencing analysis to obtain the VH:VL information. We compare the frequency of antibody variants in each population to determine the identify of neutralizing antibodies in the population^{(3,21)} as described in Example 6.

### Example 9: Application of the Assay in Emulsion Droplet Systems

In this working example, we used a microfluidic device to encapsulate the IgG expressing HEKACE2 cells in cellular secretion media to form droplets with one cell per droplet^{(6,25,26)}. An example of cell isolation and antibody secretion using CHO cells transiently transfected for antibody secretion is shown in FIG. 15.

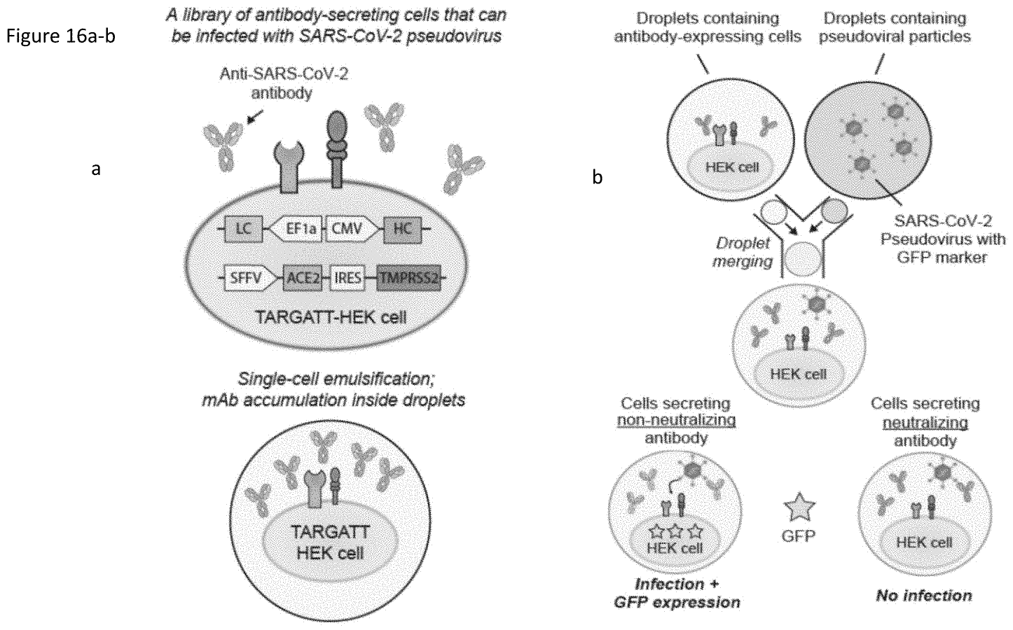

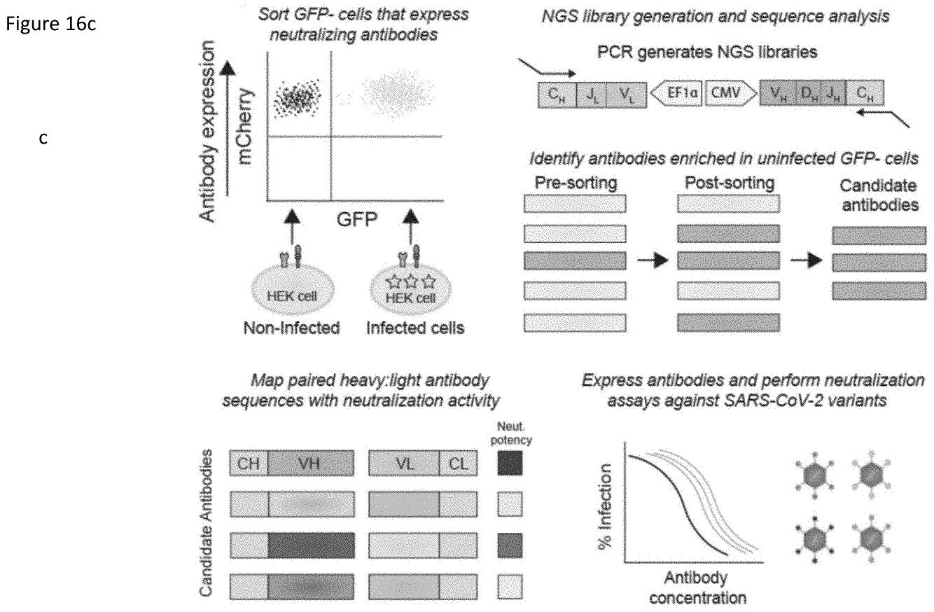

We implemented this system for SARS-CoV-2 secreted protein neutralization assays using HEKACE2 cells. The workflow for neutralization assays using cells secreting proteins inside emulsion droplets is shown in FIG. 16. We incubated the droplet for between 4 and 96 hours, although a broader time scale can also be used, allowing the secretion of the secreted protein (in this example, IgG) within the droplet. Subsequently, a second droplet containing SARS-CoV-2 pseudovirus was merged into the droplet with IgG expressing HEKACE2 cells. We used electrocoalescence to merge droplets⁽²⁴⁾, although alternative droplet merging methods are also known to persons skilled in the art and include micropillar resistance arrays. The merged droplets were further incubated for another 4-96 hours to allow pseudovirus infection or neutralization to occur, although a broader time scale can also be used. The droplets were broken, and the cells were recovered.

Droplets can be broken using chemical reagents, including 1H,1H,2H,2H-Perfluoro-1-octanol, or other methods known to individuals skilled in the art. Optionally, a potently neutralizing compound (for example, a high concentration of neutralizing antibody) can be added to the system to prevent any new pseudovirus infections after the droplets are merged together. As one salient example, the droplets are broken, and cells are recovered from droplets in the presence of high concentrations (1 mg/mL) of soluble 910-30 neutralizing IgG to prevent subsequent viral infection once cells were recovered together. Optionally, the recovered cells can be cultured for additional hours, days, weeks, or months prior to screening. Next we isolated GFP- and GFP+ cell populations using fluorescence activated cell sorting (FACS) and extracted the RNA and performed RT-PCR for the antibody genes⁽²²⁾. We will perform high-throughput sequencing analysis to obtain the VH:VL information in the GFP- and GFP+ cell groups. We will compare the frequency of antibody variants in each population to determine the identify of neutralizing antibodies in the population^{(3,21)} as described in Example 6, where GFP− cells are enriched for encoding antibodies that provide protection against SARS-COV-2 pseudovirus infection, whereas GFP+ cells are enriched for encoding antibodies that are not protective against SARS-CoV-2 or that do not express at sufficient quantities to provide protection inside droplets under the assay conditions used.

### Example 10: Neutralizing Antibody Discovery from Native Antibody Libraries^{(9,16,17,19)}

In this prophetic example, we obtain the natively paired VH:VL library using a native paired VH:VL sequencing platform. The VH:VL amplicon can be delivered as IgG or IgG fragments via random gene integration using plasmid transfection and resistance gene marker selection, as well as via site-specific integration, as described in Example 3. We screen potent SARS-CoV-2 neutralizing antibodies using multiple methods for single cell isolation, including single cell isolation into well plates (Example 7), printed chambers (Example 8), or microfluid droplets (Example 9). We then sort the GPF− and GFP+ HEKACE2 cells and perform RT-PCR to obtain paired VH:VL amplicons from each cell population. We perform PCR to add a primer barcode for next-generation sequence analysis of antibody populations, as described previously^{(16,17,19)}. We choose 10 of the most prevalent VH:VL clones enriched in the GFP negative population and for gene synthesis. We then performed transient transfection of plasmids to express IgG in a suspension of Expi293 cells. 7 day after transfection, we centrifuge the culture and transfer the supernatant into 50 mL centrifuge tube. We add 0.5 mL of protein G resin and allow the reaction to carry on a bench rotator for 2 hours. We then pour the reaction mixture into the polypropylene columns to retain the protein G resin. We then elute IgG with 0.1 M glycine-HCl, pH 2.7 and neutralize the pH with 1M Tris-HCl, pH 9.0. The purified IgGs are then concentrated and subjected to neutralization assay analysis of individual IgG. We quantified the IgG protein concentration by BCA protein assay. We then analyze the purity of the IgG by mixing 2 μg of purified IgG with SDS-page sample buffer and run through the TGX Stain-Free Precast Gel. We perform serial dilution of the antibodies from 10 μg/mL to a final concentration of 0.001 μg/mL. We combine serially diluted antibodies with 30 μL SARS-CoV-2 pseudovirus and incubate the reaction for an hour at 37° C. We then add the virus-antibody mixture into a 96 well with 2×10⁵ ACE expressing HEK293 cells. We incubate at 37° C. 5% CO₂ for three days. We analyze the antibodies' neutralization activity via flow cytometry analysis of the GFP signal to demonstrate the recovery of neutralizing antibodies that are enriched in the GFP− cell population

### Example 11: Antibody Library Variant Expression and Directed Evolution Selection for Potent Neutralizing Antibodies

In this prophetic example, we mutate an anti-SARS-CoV-2 antibody by one of the methods from DNA shuffling, error prone-PCR, single-site directed mutagenesis to generate antibody variant libraries. To increase the mutational landscape, we can further perform combinatorial and/or sequential mutation. We clone the synthetic antibody mutant libraries into HEKACE2 cells described in Example 3. We screen the SARS-CoV 2 neutralizing antibodies by one of the approaches delineated in Example 7, Example 8 and Example 9. After the sorting of GFP− and GFP+ cells and subsequent recovery of the VH:VL sequences information via RT-PCR from the GFP-negative IgG-expressing HEKACE2 cells (enriched for secretion of neutralizing antibodies), we re-deliver the screened VH:VL gene into the IgG expressing vectors detailed in Example 3 (named enriched IgG libraries) for subsequent rounds of screening. We can also perform sequential mutation using DNA shuffling, error prone-PCR, single site directed mutagenesis to enhance the diversity between screening rounds; other DNA sequencing and library diversity generation strategies can also be used and are known to those skilled in the art. We express both enriched libraries and enriched plus mutated IgG libraries on an IgG expressing platform, as presented in Example 3. We re-screen and obtain the neutralizing antibodies VH:VL sequences using methods described in Example 7, Example 8 or Example 9. We repeat the re-delivery and screen of enriched libraries for subsequent rounds to further enrich for neutralization potency, until a molecule with the desired neutralization potency is obtained. This process of re-screening, mutation, and re-delivery enables directed evolution selection for potently neutralizing antibodies.

### Example 12: Antibody Variant Neutralization of Many Viral Strains Sequentially

In this prophetic example, we use SARS-CoV-2 Wuhan Hu-1 strain as our pseudovirus for neutralization analysis to isolate the neutralizing antibodies from method described in Examples 10 and 11. We can recover the neutralizing IgG libraries expressing HEKACE2 cells through recovering the GFP− cells. We use another virus mutation variant such as S-D614G variant to perform sequential neutralization screening (Defined as second round) via cell isolation platforms as described in Example 7, Example 8 or Example 9. After the second round of screening, the populations are enriched for antibodies that exhibit neutralizing capabilities against both Wuhan Hu-1 and D614G.

Alternatively, after the FACS post sorting of GFP negative cells we extract the RNA from these cells and perform RT-PCR to obtain the VH:VL sequences. We then re-deliver the VH:VL pair into the HEKACE2 cells described at Example 3 to generate the secreted protein library after a single library sort. We then perform the second round of screening using the pseudovirus variant contain D614 mutation. These methods allow for the selection of neutralizing antibodies targeting multiple viral strains of interest.

### Example 13: Antibody Variant Neutralization with Many Viral Strains Concurrently

In this prophetic example, perform the pseudovirus neutralization using multiple virus strains at the same time. We first mix an equal amount of the virus from broad coronavirus strains, including SARS-CoV-2, SARS-Cov-2-D614G, SARS-CoV-1, MERS-CoV, with each pseudovirus contain YFP, GFP, DsRed and CFP, respectively. Alternatively, we can use different viral strains all derived from different SARS-CoV-2 variants (e.g., B.1.1.7, B.1.351, P.1, B.1.427, and B.1.429). In some embodiments, all viruses encode for the same reporter (e.g., GFP). In some embodiments, each virus encodes for a different DNA or RNA barcode that the target cells will express after infection. In some embodiments, authentic virus is used. In other embodiments, pseudovirus is used. We perform neutralization assays of antibody libraries with mixture of viral strains based on approaches described in Example 7 (multiple well plates based), Example 8 (microchamber based) or Example 9 (microfluidic droplet based). For multiple-well plate assay in Example 7, we choose the well-containing cells showed no YFP, GFP, DsRed and CFP as candidate cells that express antibodies with broad neutralization. Other fluorescent markers can be used and are known to those skilled in the art. In both microchamber (Example 8) and microfluidic droplet-based methods (Example 9), after we retrieve cells from either microchambers (Example 8) or droplets (Example 9), we rest and expand cells for another 48 hours (the cells can be rested for any amount of time between 0 hours and multiple months depending on the experimental preference). We sort the cells with no YFP, GFP, DsRed and CFP expression, and also the cells that show fluorophore expression (i.e., were infected). We obtain the VH:VL pairing information of each population through RT-PCR gene recovery and high-throughput sequencing. We compare the sequences of both screening populations, and we then express the candidate antibodies enriched in the populations no YFP, GFP, DsRed and CFP from the HEK293Expi cells and purify the antibody for quantification. We evaluate individual antibody's neutralization capability against SARS-CoV-2, SARS-Cov-2-D614G, SARS-CoV-1, and MERS-CoV according to the method described in Example 10.

### Example 14: Antibody Variant Neutralization with Many Different Viruses Concurrently

In this prophetic example, we perform the pseudovirus neutralization using multiple different virus types at the same time. We first mix an equal amount of the virus from different strains, including SARS-CoV-2, SARS-Cov-2-D614G, YFV, and DENV-1, with each pseudovirus contain YFP, GFP, DsRed and CFP, respectively. In some embodiments, all viruses encode for the same reporter (e.g., GFP). In some embodiments, each virus encodes for a different DNA or RNA barcode that the target cells will express after infection. In some embodiments, authentic virus is used. In other embodiments, pseudovirus is used. A cell line is generated that can be infected by any of the viruses used. In some embodiments, a cell that can be infected with SARS-CoV-2, SARS-Cov-2-D614G, YFV, and DENV-1 is generated by starting with Raji-DC-SIGN cells, which are used for in vitro infections with YFV and DENV-1 recombinant viral particles (RVPs) and modify Raji-DC-SIGN to express the ACE2 protein that enables infection also with SARS-CoV-2. We next clone a library of antibodies to express and secrete antibody from the modified Raji-DC-SIGN-ACE2 cells and perform neutralization assays of antibody libraries with mixture of viruses based on approaches described in Example 7 (multiple well plates based), Example 8 (microchamber based) or Example 9 (microfluidic droplet based). For multiple-well plate assay in Example 7, we choose the well-containing cells showed no YFP, GFP, DsRed and CFP as candidate cells that express antibodies with broad neutralization. In both microchamber (Example 8) and microfluidic droplet-based methods (Example 9), after we retrieve cells from either microchambers (Example 8) or droplets (Example 9), we rest and expand cells for another 48 hours (although cells can be rested from anywhere in between 0 hours and multiple months, depending on the preferences of the experiment). We sort the cells with no YFP, GFP, DsRed and CFP expression, and also the cells that show fluorophore expression (i.e., were infected). We obtain the VH:VL pairing information of each population through RT-PCR gene recovery and high-throughput sequencing. We compare the sequences of both screening populations, and we then express the candidate antibodies enriched in the populations no YFP, GFP, DsRed and CFP from the HEK293Expi cells to determine the sequences of neutralizing antibodies using a high-throughput assay. In some embodiments, each virus encodes a cell-specific barcode that encodes for the virus type, allowing for a high-throughput DNA-based readout of the infecting viruses in the library of bulk or single cells, in addition to high-throughput analysis of the antibody gene sequences in the infected or non-infected antibody populations. In some embodiments, single cell sequencing is used to link the barcode of the infecting virus to the DNA sequence of the antibody directly.

### Example 15: Rapid High-Throughput Discovery of Secreted Proteins Activating 4-1BB

In this prophetic example, a cell line is used for 4-1BB expression along with a reporter that causes expression of a fluorescent marker or other reporter (for example, GFP or other cellular selection markers known in the art) when 4-1BB is activated. A fusion protein of 4-1BB extracellular domain is generated with an internal activation signal that causes GFP expression when 4-1BB is activated. A protein library is encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). In some embodiments, the compartments are comprised of emulsion droplets. The cells that secrete protein that activate 4-1BB will activate fluorescent marker expression (for example, GFP). After cell recovery, the marker+ and marker− cells are isolated via flow cytometry, and their identities characterized by DNA sequencing to determine the protein variants within the library that can functionally activate 4-1BB. As an alternative approach, a luciferase detection system could be used in place of fluorescent cell sorting to detect secreted proteins with functional activities of interest. After the identification of appropriate secreted proteins with functional activities of interest, the discovered proteins would have a potential as immunotherapies to activate 4-1BB for the treatment of cancer or other diseases.

### Example 16: Rapid High-Throughput Discovery of Secreted Proteins Blocking Programmed Death Receptor 1 (PD-1) Activation

In this prophetic example, a cell line is generated for PD-1 expression along with a selectable marker that causes expression of a fluorescent reporter or other reporter (for example, GFP or other cellular selection markers known in the art) when PD-1 is activated. A fusion protein of PD-1 extracellular domain is generated with an internal activation signal that causes GFP expression when PD-1 is activated. A protein library is encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). In some embodiments, the compartments are comprised of emulsion droplets. Then, PD-L1 is added to the compartments to induce the ligation and activation of PD-1. The cells that secrete protein that blocks PD-L1 binding and/or prevents PD-1 activation will prevent the fluorescent marker from being expressed. After cell recovery, the GFP− cells are isolated via flow cytometry, and their identities characterized by DNA sequencing to determine the protein variants within the library that can block PD-1 activation via PD-L1. As an alternative approach, a luciferase detection system could be used in place of fluorescent cell sorting to detect secreted proteins with functional activities of interest. After the identification of appropriate secreted proteins with functional activities of interest, the discovered proteins would have a potential ability to be immunotherapeutic checkpoint inhibitors for cancer treatment.

### Example 17: Rapid High-Throughput Discovery of Secreted Proteins BLOCKING GPCR Activation

In this prophetic example, a cell line is generated for G protein coupled receptor (GPCR) expression along with a reporter that causes expression of a fluorescent marker (or other reporter (for example, GFP or other cellular selection markers known in the art) when the GPCR is activated. A GPCR is expressed in a cell line that activates an internal activation signal when the GPCR is activated. Example cell lines are commercially available, such as through the Eurofins DiscoverX company that vends GPCR cell lines or can be similarly built. A secreted protein library is also encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). In some embodiments, the compartments are comprised of emulsion droplets. Then, a GPCR agonist is added to the compartments to induce the ligation and activation of the GPCR. The cells that secrete protein that blocks GPCR agonist binding and/or prevents GPCR activation will prevent the fluorescent marker from being expressed. After cell recovery, activated- and non-activated cells are isolated via flow cytometry, and their identities characterized by DNA sequencing to determine the protein variants within the library that can block GPCR activation. As an alternative approach, a luciferase detection system could be used in place of fluorescent cell sorting to detect secreted proteins with functional activities of interest. After the identification of appropriate secreted proteins with functional activities of interest, the discovered proteins would be promising candidates as drugs to block GPCR activation.

### Example 18: Rapid High-Throughput Discovery of Secreted Proteins Inducing GPCR Activation

In this prophetic example, a cell line is generated for G protein coupled receptor (GPCR) expression along with a reporter that causes expression of a fluorescent reporter or other reporter (for example, GFP or other cellular reporters known in the art) when the GPCR is activated. A GPCR is expressed in a cell line that generates an internal activation signal that when the GPCR is activated. Example cell lines are commercially available, such as through the Eurofins DiscoverX company that vends GPCR cell lines or can be similarly built. A secreted protein library is encoded in the cell line (one protein variant per cell) that causes each cell to secrete the protein variant. The cells are isolated as single cells inside compartments and allowed to incubate for 4 hours to accumulate secreted protein (although the time can range from seconds to months, depending on the conditions and goals of the experiment). The cells that secrete protein that activate GPCR will cause the fluorescent marker to be expressed in those same cells. After cell recovery, activated- and non-activated cells are isolated via flow cytometry, and their identities characterized by DNA sequencing to determine the protein variants within the library that activate GPCRs. As an alternative approach, a luciferase detection system could be used in place of fluorescent cell sorting to detect secreted proteins with functional activities of interest. After the identification of appropriate secreted proteins with functional activities of interest, the discovered proteins would be promising candidates as drugs to activate GPCRs.

### Example 19: Secreted Protein Analysis for Neutralization of Yellow Fever Virus

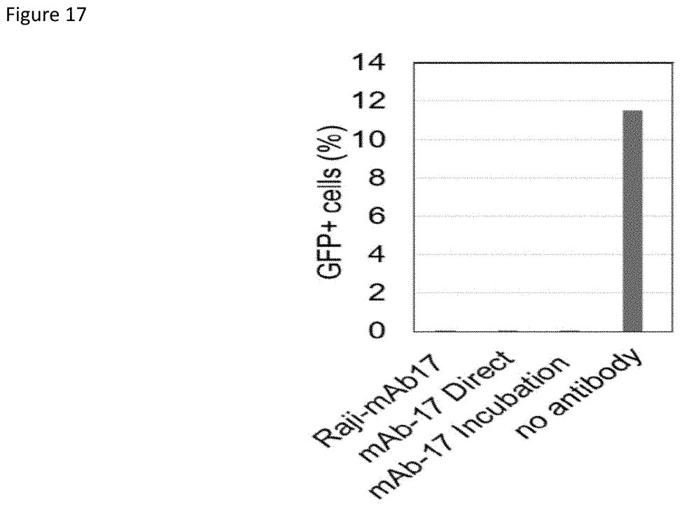

In this working example, Raji-DCSIGNR cells were used to test the ability of secreted proteins to neutralize yellow fever virus (YFV). In this example we expressed antibody in the bi-cistronic format using a p2a motif as described in Example 5, using lentiviral transduction to insert genes into the cells for secretion. We utilized lentiviral transduction of Raji-DCSIGNR cells to evaluate their capacity for high-throughput single-cell neutralization assays. We transduced Raji-DCSIGNR cells⁽¹⁶⁾ with a yellow fever virus neutralizing antibody, mAb-17, for antibody secretion, or with an empty plasmid that would not induce antibody expression. We tested the cell lines after 4 days of antibody secretion in 96-well plates before adding YFV recombinant viral particles (RVP) to verify that the secreted antibody would provide protection from YFV RVP (FIG. 17). The cells expressing mAb 17 were protected from YFV RVP infection, whereas cells that were not expressing mAb 17 were unprotected from infection. These data confirmed that we can link antibody secreted protein functional neutralization properties to a GFP-based reporter (that is expressed after the RVP infection event) as a cell line platform for direct screening of anti-YFV antibody neutralization in a rapid, high-throughput manner.

### Example 20: Antibody Expression with Different Leader Peptide and Promoter Combinations

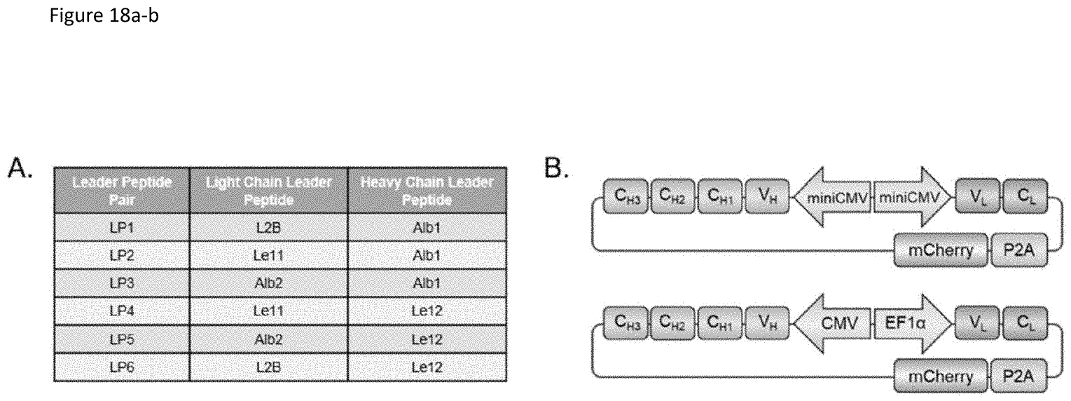

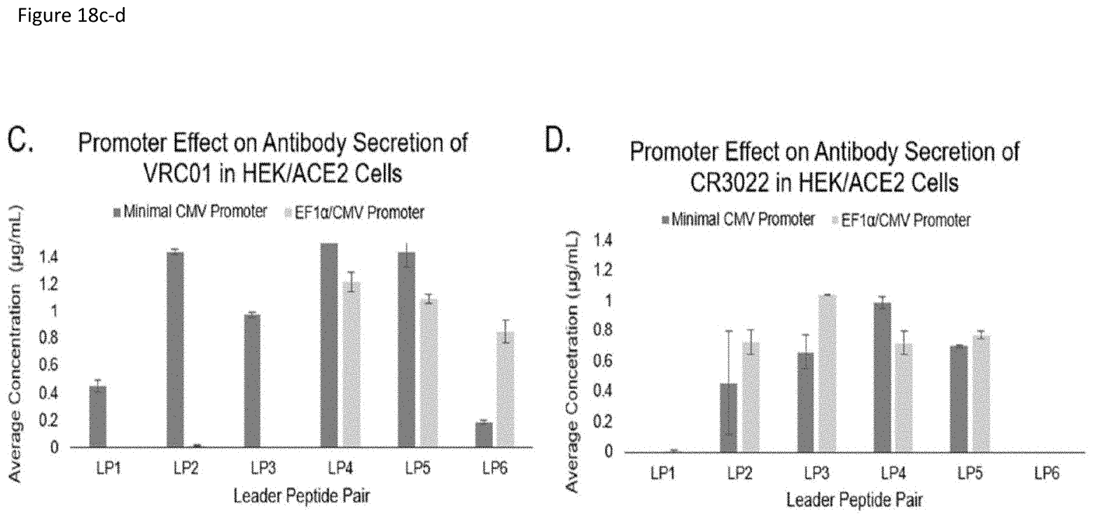

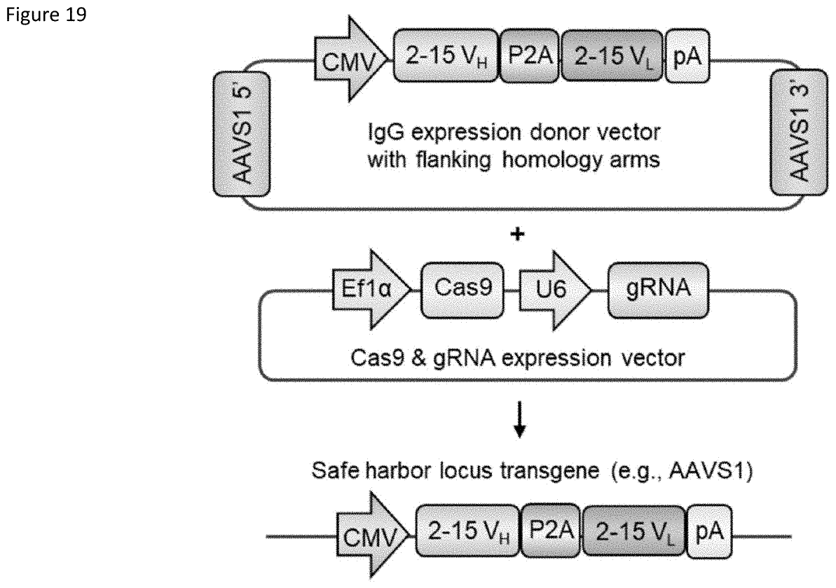

In this working example we tested the ability of HEK293 cells expressing ACE2 to be transiently transfected with plasmids containing 910-30 expressed with different leader peptide combinations (LP1, LP4, LP5 and LP6) for antibody secretion. Lipofectamine 3000 was used as a transfection reagent following the reverse transfection protocols in 96 well plates and incubated for two days at 37° C. Two days post-transfection, 40 μL of SARS-CoV-2 pseudovirus with GFP reporter gene was added to the cells and incubated at 37° C. for another three days. Three days after adding the pseudovirus, the supernatant containing secreted IgG is removed from the cells for use in ELISA antibody quantification. The ELISA readout is illustrated in FIG. 18. Antibodies VRC01 and CR3022 expressed by numerous peptide combinations showed successful antibody expression, indicating the successful secretion of antibodies using different leader peptides and promoter sequences.

### Example 21: Use of CRISPR-Cas9 to Clone Antibodies into Soluble Protein Cell Secretion Platforms

In this working example, we cloned an anti-SARS-Cov2 monoclonal antibody, 2-15, into a donor vector AAVS1 Safe Harbor Targeting Knock-in HR Donor 2 vector, GE622A-1, from System Biosciences. We named the donor plasmid with 2-15 monoclonal antibody, pGE622A2-15. We then co-transfected the 2-15 donor plasmid (pGE622A2-15) and the All-in-one Cas9 Smart Nuclease AAVS1 Targeting Plasmid (System Bioscience #CAS601A-1) into the Expi293 cells. The expression of the Cas9 nuclease and the gRNA after the transfection generated a double strain break at the Expi293 cell AAVS1 genome site. 2-15 gene sequence from the donor plasmid was integrated into the AAVS1 gene locus because of homologous recombination event (See illustration of the 2-15 gene integration below). We began the puromycin selection (at a concentration of 5 μg/mL) 1-week post-transfection to reduce the random integration Expi293 cells. The stable cell pool with 2-15 gene integration was named Expi2-15.

We seeded the Expi2-15 and Expi293 cells into a 96-well plate with a density of 3.2×10⁴ cells per well. We then transfected the cell with ACE2/TMRPSS2 expressing plasmid right after seeding these cells (both Expi2-15 and Expi293). For the positive control of the neutralization assay, we added purified 91030 antibody (5 μg/mL of final concentration) into ACE2/TMRPSS2 expressing Expi293 cells. We aliquoted 20 μL of the culture media from each well for further IgG quantification analysis. We added 80 μL of the SARS CoV-2 reporter virus particle with spike protein D614G mutation and luciferases reporter gene to the ACE2/TMPRSS2 expressing Expi2-15 cells (ACE2/TMPRSS2+ Expi2-15), ACE2/TMPRSS2 expressing wild-type Expi293 cells (ACE/TMPRSS2+ Expi293) and ACE/TMPRSS2 expressing wild-type Expi293 cells with 5 μg/mL of 91030 (ACE/TMPRSS2+ Expi293+91030).

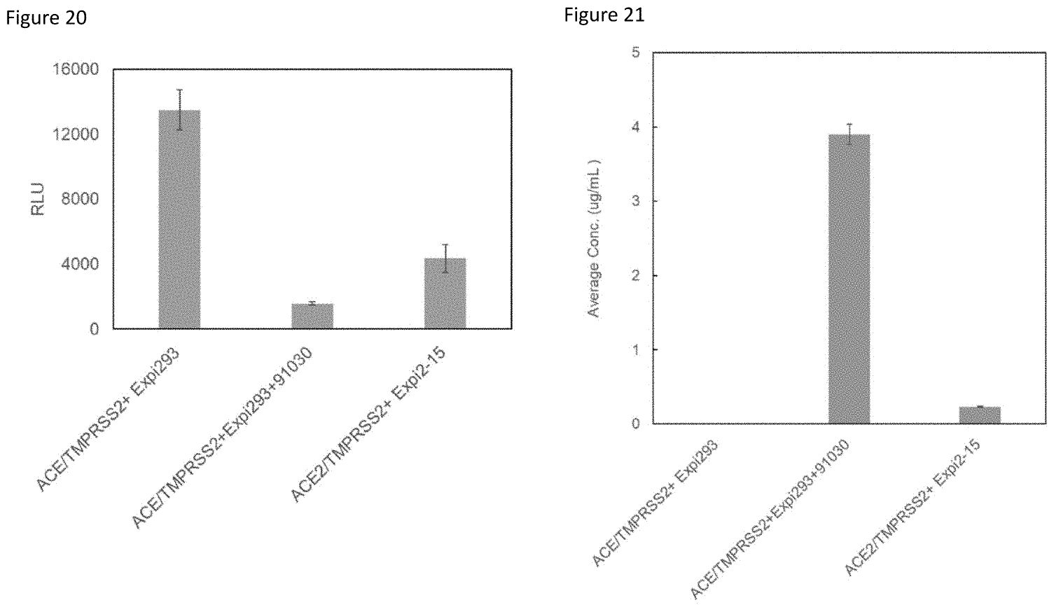

Three days post adding the reporter virus, we removed the culture media and added 30 μL of PBS and 30 μL of diluted Renilla-Glo Assay Substrate (diluted Renilla-Glo Assay Substrate into the Assay Buffer at 1:100). We then detected luminescence in a luminometer after 10 minutes of incubation at room temperature. We calculated the average relative light unit (RLU) of the luminometer reading of each group. As shown in the figure below, both ACE2/TMPRSS2+ Expi2-15 and ACE/TMPRSS2+ Expi293+91030 (Positive Control) groups showed a significant reduction in relative light units as compared with that of the ACE/TMPRSS2+ Expi293 group, indicating that 2-15 secreted from Expi2-15 cells is able to neutralize the SARS-CoV2 pseudovirus (FIG. 20).

We validated the antibody expression level of the ACE2/TMPRSS2+ Expi2-15 group via ELISA. The average antibody expression level is 0.23 μg/mL (n=6) antibody expression from the ACE/TMPRSS2+ Expi2-15, suggesting that Expi2-15 cells are able to secret functionally active 2-15 (FIG. 21). The group, ACE/TMPRSS2+Expi293+91030, containing 5 ug/mL of the purified 91030 monoclonal antibody (calculated based on nanodrop of the purified 91030) was measured at a concentration of 3.9 μg/mL via ELISA as internal control for our ELISA assay.

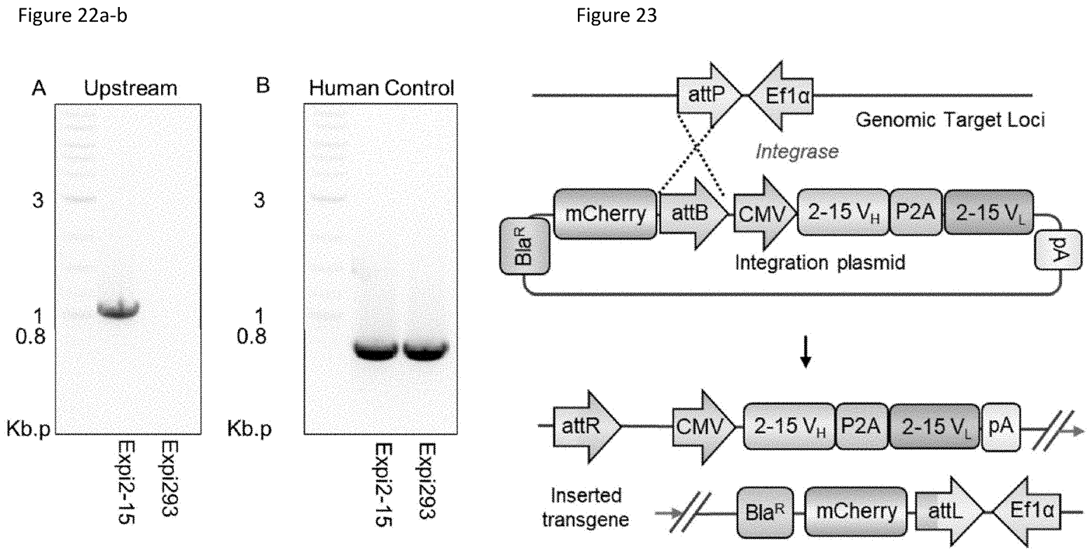

We further performed genomic PCR to validate the integration of the 2-15 mab gene sequencing into the Expi2-15 cell line. We first isolated the genomic DNA from wild-type Expi293 and Expi2-15 cell lines. Then we performed PCR amplification to amplify the upstream gene integration region using GoTaq2 hot-start polymerase (Promega #M7405) and primers (Upstream primer set, Forward 5′ TCCTGAGTCCGGACCACTTT 3′ (SEQ ID NO: 25) and Reverse 5′ CACCGCATGTTAGAAGACTTCC 3′ (SEQ ID NO: 26)) validated and provided by the System Bioscience. A 1000 b.p. amplicon from the Expi2-15 cells indicated a successful gene integration compared with no PCR amplification from the wild-type Expi293 cells (see FIG. 22a).

A separate PCR reaction using a human control primer set (Forward 5′-ACCTCCAGTTAGGAAAGGGGACT-3′ (SEQ ID NO: 27) Reverse 5′-AAGTTTTTCTTGAAAACCCATGGAA-3′ (SEQ ID NO: 28)) for internal PCR control (FIG. 22b).

### Example 22: Use of TARGATT Specific Integration to Clone Antibodies into Soluble Protein Cell Secretion Platforms

In this working example, we followed the instruction manual of the TARGATT™ HEK master cell line knock-in kit to clone an anti-SARS-Cov2 monoclonal antibody, 2-15, into the TARGATT 24 CMV-MCS-attB (named pTARGATT2-15) to produce the donor plasmid. We then co-transfected the 2-15 donor plasmid (pTARGATT2-15) and the integrase plasmid into TARGATT HEK master cells. The integrase catalyzes a gene recombination event allowing the integration of 2-15 monoclonal antibody, mCherry and blasticidin selectable marker into genome (See FIG. 23).

Three days after transfection, we sub-cultured the transfected cell with a split ratio of 1:20. Twenty-four hours after the sub-culture, we added blasticidin in a concentration of 10 μg/mL and maintained blasticidin selection pressure for two weeks. We then performed cell sorting to isolate the mCherry positive cells to enrich the 2-15 integrated cells (named TARGATT2-15). After the recovery of the TARGATT2-15 cells, we seeded the TARGATT2-15 and wild-type TARGATT cells into a 96-well plate with a density of 3.2×10⁴ cells per well. We then transfected the cell with ACE2/TMRPSS2 expressing plasmid right after seeding the cells (as described in Example 1 and in FIG. 2). Two days after the transfection, we added purified 91030 antibody (5 μg/mL of final concentration) into unmodified TARGATT cells as positive control prior to the pseudovirus neutralization assay. We aliquoted 20 μL of the culture media from each well for further IgG quantification analysis.

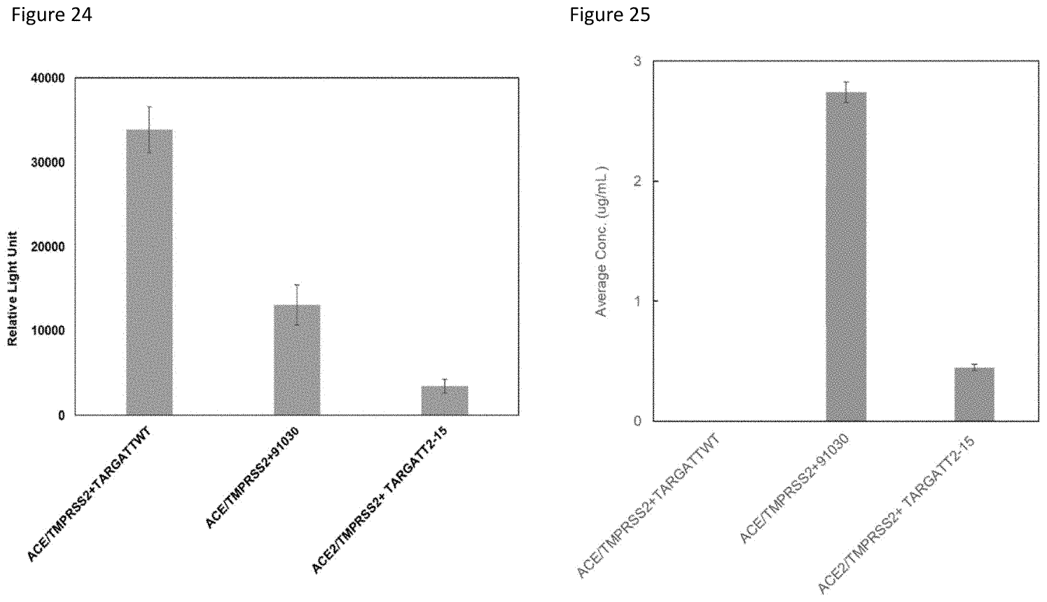

We added 80 μL of the SARS CoV-2 reporter virus particle with spike protein D614G mutation and luciferases reporter gene to the ACE2/TMPRSS2 expressing TARGATT2-15 cells (ACE2/TMPRSS2+TARGATT2-15), ACE2/TMPRSS2 expressing wild-type TARGATT cells (ACE/TMPRSS2+TARGATTWT) and ACE/TMPRSS2 expressing wild-type TARGATT cells with 5 μg/mL of purified 91030 (ACE/TMPRSS2+91030). Three days post adding the reporter virus, we removed the culture media and added 30 μL of PBS and 30 μL of diluted Renilla-Glo Assay Substrate (diluted Renilla-Glo Assay Substrate into the Assay Buffer at 1:100). We then detected luminescence in a luminometer after 10 minutes of incubation at room temperature. We calculated the average relative light unit (RLU) of the luminometer reading of each group. As shown in the figure below, both ACE2/TMPRSS2+2-15 and ACE/TMPRSS2+91030 groups showed a significant reduced in relative light unit as compared with that of the ACE/TMPRSS2+WT group, indicating that 2-15 secreted from TARGATTHEK2-15 cells is able to neutralize the SARS-CoV2 pseudovirus (FIG. 24).

We validated the antibody expression level of the ACE2/TMPRSS2+ TARGATT2-15 group via ELISA. The average antibody expression level is 0.44 μg/mL (n=6) antibody expression from the TARGATT2-15, demonstrating that TARGATT2-15 cells are able to secret functionally active 2-15 (see FIG. 25). The group, ACE/TMPRSS2+91030, containing 5 μg/mL of the purified 91030 monoclonal antibody (calculated based on nanodrop of the purified 91030) was measured at a concentration of 2.74 μg/mL via ELISA as internal control for our ELISA assay.

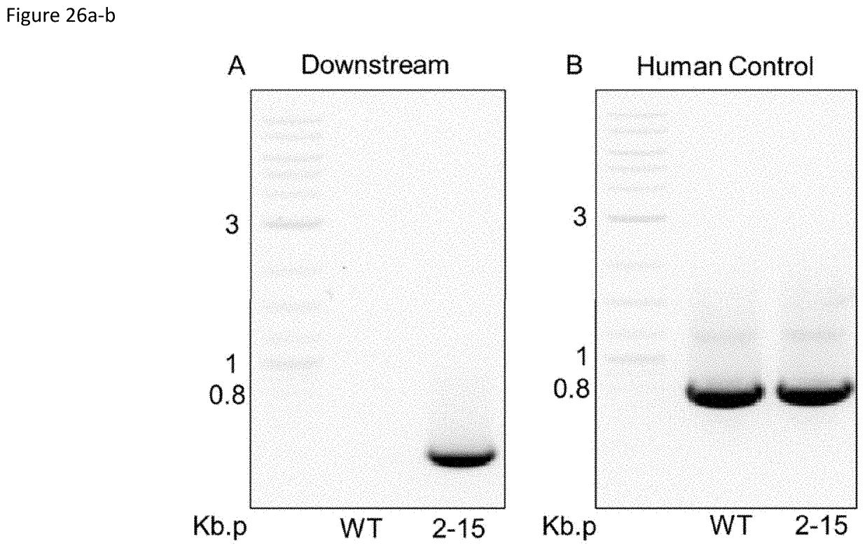

We further performed genomic PCR to validate the integration of the 2-15 mab gene sequencing into the TARGATT2-15 cell line. We first isolated genomic DNA from wild-type TARGATT and TARGATT2-15 cell lines. Then we performed PCR amplification to amplify the downstream gene integration region using GoTaq2 hot-start polymerase (Promega #M7405) and primer sequences (Downstream primer set, Forward 5′ CCTTGTAGATGAACTCGCCGT 3′(SEQ ID NO: 29) and Reverse 5′ GGTGTCGTGATTATTCGAAGGG 3′(SEQ ID NO: 30)) validated and provided by the Applied StemCell, Inc. A 500 b.p. amplicon from the TARGATT2-15 group indicated a successful gene integration compared with no PCR amplification from the wild-type TARGATT cells (FIG. 26).

### Example 23: Use of Rapid Droplet-Based Assays to Identify Neutralizing Antibodies Using Next-Generation Sequencing

In this prophetic example, we clone antibodies into Raji-DCSIGNR cells and generate a synthetic library mixture to test the ability of droplet-based screening to identify neutralizing antibodies targeting yellow fever virus (YFV). We utilize lentiviral transduction of Raji-DCSIGNR cells to evaluate their capacity to be used in high-throughput single-cell neutralization assays. We transduce Raji-DCSIGNR cells with a yellow fever virus neutralizing antibody, mAb-17, for antibody secretion, or with other antibodies (910-30, VRC01, and 2-15) that do not neutralize YFV. We encapsulate the cells in microfluidic droplets and incubate them for 24 hours (although the incubation time could range from minutes to several weeks depending on the goals of the experiment) to facilitate antibody secretion and accumulation within the droplet. Next, we merge the droplets using the electrocoalescence technique (other techniques for droplet merging can also be used and are known to those skilled in the art) and incubate overnight at 37 degrees Celsius to allow the pseudovirus to infect any cells that are not protected by secreted antibodies (the amount of incubation time and the temperature of incubation can vary according to the goals of the experiment). Droplets are broken, and the cells are recovered. After a brief incubation time (which can range from 0 minutes to several weeks depending on the goals of the experiment), we sort GFP+ and GFP− cells on a flow cytometer to separate the neutralizing and non-neutralizing cells. Cells are collected and genomic DNA is extracted for PCR-based amplification.

DNA is sent for next-generation sequencing to quantify the prevalence of each antibody clone in the dataset. The neutralizing antibodies are enriched in the set of GFP− cells, and depleted in the GFP+ cells, and neutralizing antibodies could be identified based on these enrichment features. These data will confirm that we can link antibody secreted protein functional neutralization properties to a reporter (that is expressed after the recombinant viral particle, RVP, infection event) as a cell line platform for direct screening of anti-YFV antibody neutralization in a rapid, high-throughput manner, and furthermore, that the sequences of neutralizing antibodies can be detected using next-generation sequencing analysis.

### Example 24: Secreted Protein Analysis for Neutralization of HIV-1

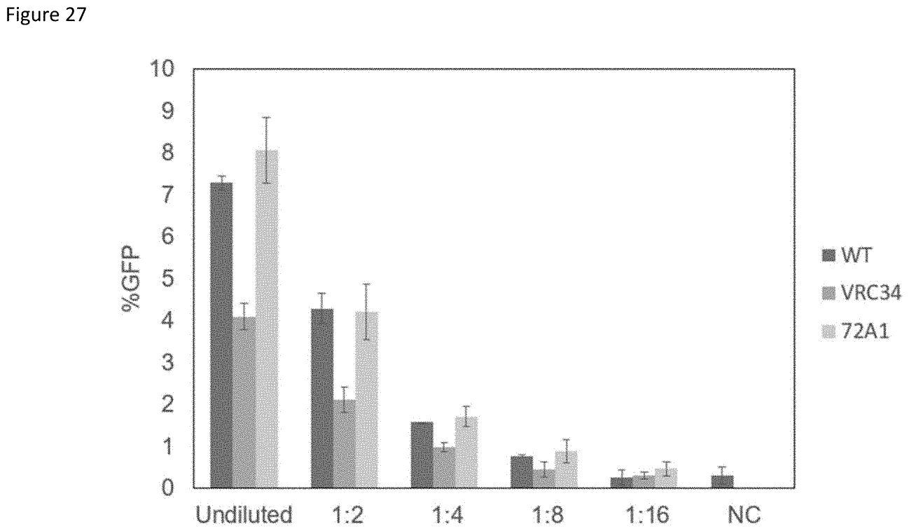

In this working example, TZM-GFP cells were used to test the ability of secreted proteins to neutralize human immunodeficiency virus 1 (HIV-1). We utilized lentiviral transduction TZM-GFP cells to evaluate their capacity for high-throughput single-cell neutralization assays. We transduced TZM-GFP cells⁽²³⁾ with an HIV-1 neutralizing antibody, VRC34, for antibody secretion, or with a control antibody that does not neutralize HIV-1 (72A1) We tested the cell lines after 2 days of antibody secretion in 96-well plates before adding HIV-1 pseudovirus particles (strain W6M.EnV.C2) to verify that the secreted antibody would provide protection from HIV-1 pseudoviruses (FIG. 27). The cells expressing VRC34 were protected from HIV-1 pseudovirus infection, whereas cells that were not expressing VRC34 were unprotected from infection. These data confirmed that we can link antibody secreted protein functional neutralization properties to a GFP-based reporter (that is expressed after the pseudovirus infection event) as a cell line platform for direct screening of anti-HIV-1 antibody neutralization in a rapid, high-throughput manner.

### Example 25: Droplet Merging Techniques to Enable Soluble Secretion Assays Inside Droplets with a Secreted Protein Cell Library

In this working example, we applied droplet merging techniques to demonstrate the encapsulation and droplet merger, and the recovery of DNA from cell libraries, to enable secretion cell assays. We first generated a synthetic cell library, where each cell secretes a separate antibody clone and also expresses ACE2, that could be used to screen for secreted protein function. We mixed four different cell groups expressing antibody clones into a single library (Table 3).

Table 3. Cells expressing known antibody clones were mixed and used as artificial cell libraries. HEK293-T clones expressing ACE2 and different monoclonal antibody clones were mixed as shown at 1×10⁶ cells/mL in High glucose DMEM supplemented with 5% fetal bovine serum and 1% penicillin-streptomycin.

TABLE 3

| Clone | Cell number | % of library |
|---|---|---|
| HEK/ACE2 VRC01 | 3,960,000 | 99% |
| HEK/ACE2 CR3022 | 13,333 | 0.33% |
| HEK/ACE2 910-30 | 13,333 | 0.33% |
| HEK/ACE2 1-20 | 13,333 | 0.33% |
| Total | 4,000,000 | 100% |

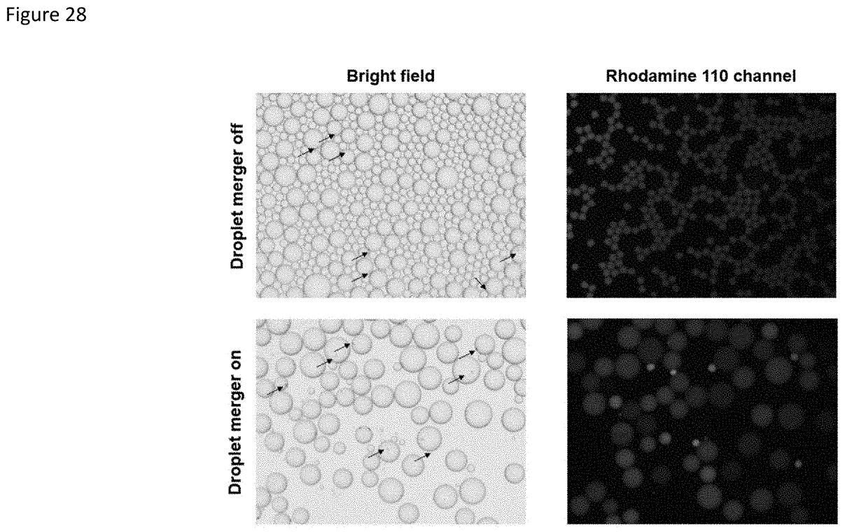

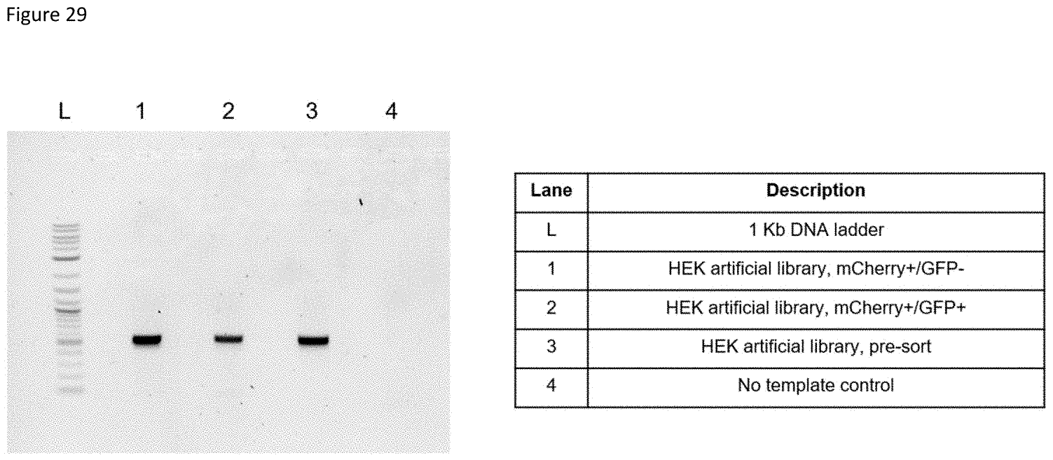

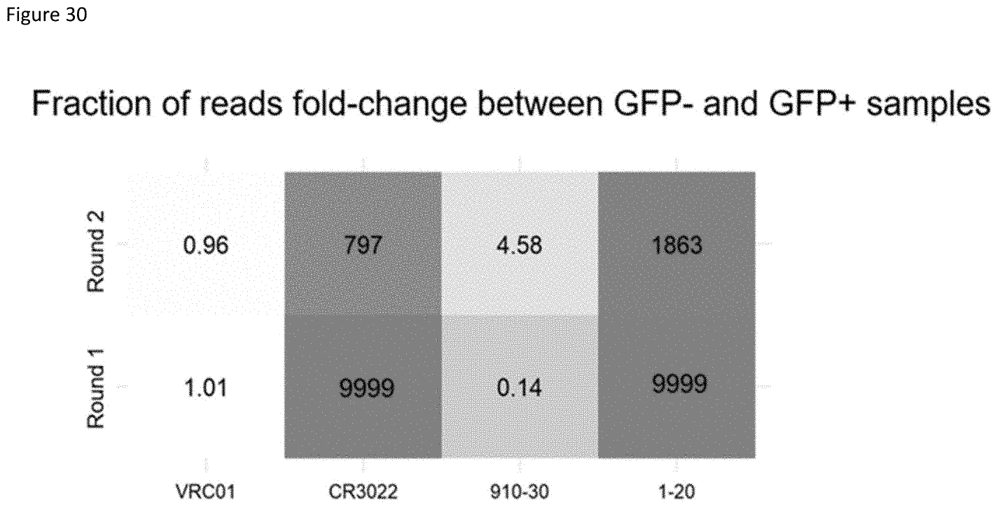

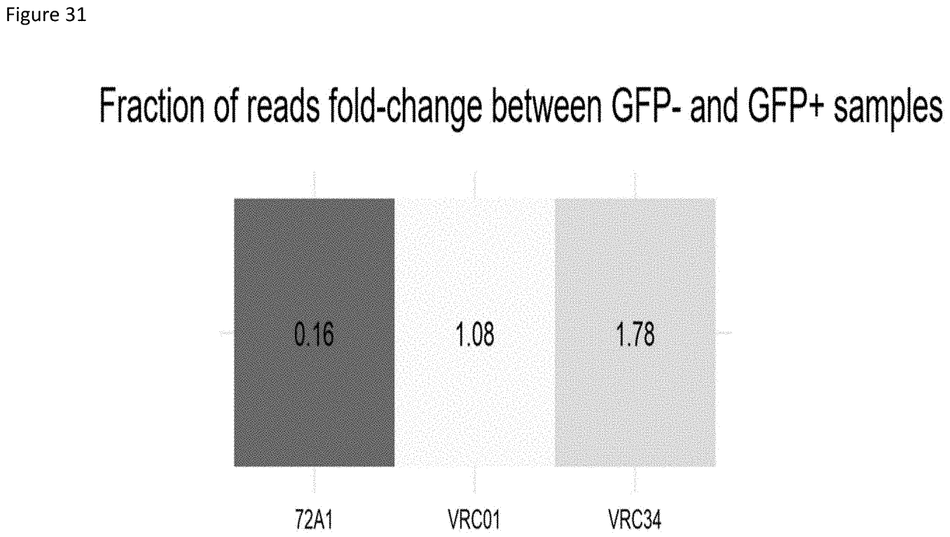

Cells were captured into single cell emulsions using a droplet generator (F02-HPB-8x, uFluidix, Canada), which generates ˜80 μm diameter droplets. Next, droplets were loaded into a droplet merging device that applies an electric field to induce the merging of droplets. This device also generates droplets containing rhodamine 110 (diameter: ˜40 μm. #83695, Sigma-Aldrich, USA) for merging with the cell droplets. (FIG. 28).

### Example 26: Recovery of DNA to Identify Secreted Proteins in Cell Populations Sorted with Different Selection Markers after Soluble Protein Secretion Assays

In this working example, we applied droplet merging techniques to demonstrate the encapsulation and droplet merger, and the recovery of DNA from cell libraries, to enable secretion cell assays. We generated and sorted a synthetic cell library (Table 3), with four different antibody clones, only some of which can potently neutralize SARS-CoV-2.

After droplet merger with SARS-CoV-2 pseudovirus that induces GFP expression in infected cells, cells were recovered from emulsions and sorted for expression of the GFP marker that indicates functional performance differences among the secreted antibodies in the library. In this case, the functional screen identified neutralizing antibodies, comparatively enriched in the GFP− cell population, and non-neutralizing antibodies were contained in the GFP+ cell population.

Genomic DNA was isolated from HEK cells using Quick-DNA Miniprep Kit (Zymo Research, USA). Next, heavy chain variable regions were amplified using Platinum Taq DNA Polymerase (ThermoFisher Scientific, USA) using primers anchoring the 3′ region of the cytomegalovirus promoter and the 5′ region of the heavy constant chain. The primer sequences used were: Forward: 5′-GGTGGGAGGTCTATATAAGCA-3′ (SEQ ID NO: 31), Reverse: 5′-CCAGAGGTGCTCTTGGAG-3′ (SEQ ID NO: 32). Polymerase chain reaction was carried out during 40 cycles using 51° C. as annealing temperature. PCR products were resolved in a 1% agarose gel, using a 1 Kb DNA ladder (#N05505, New England BioLabs, USA) to control for size. The resulting DNA gels are shown in FIG. 29. These data demonstrate our ability to recover the DNA sequences from cells utilized in high-throughput droplet-based cell secretion protein functional assays.

### Example 27 Application of the Single-Cell Assay Using a Synthetic Library of Antibodies with Known Neutralization Properties Against SARS-CoV-2 Inside Emulsion Droplet Systems

This working Example relates to the successful screening of a synthetic cell library secreting antibody molecules for the neutralization of SARS-CoV-2 pseudovirus. First, we mixed HEK-ACE2 expressing different monoclonal antibodies to generate a synthetic library consisting of 4 antibody-producing cells (the previously reported antibodies VRC01, CR3022, 910-30 and mAb1-20); VRC01 does not neutralize SARS-CoV-2 and serves as a negative control. We used a microfluidic device to encapsulate the synthetic library with DMEM media to form droplets, with one cell per droplet. We incubated the droplet for 24 hours, allowing the secretion of IgG within the droplet for antibody accumulation. Subsequently, a second droplet containing D614G SARS-CoV-2 pseudovirus was merged into the droplet with IgG expressing HEK-ACE2 cells. We used electrocoalescence to merge droplets, although alternative methods to merge droplets have been reported including the use of micropillar resistance arrays. The merged droplets were further incubated for another 24 hours to allow pseudovirus infection or neutralization to occur. The droplets were then broken, and the cells are recovered. Cells were allowed to recover for 48 hours. We isolated GFP− and GFP+ populations using fluorescence activated cell sorting (FACS) and extracted the gDNA from cell aliquots and performing PCR to recover the antibody gene libraries for NGS analysis. GFP− cells were also recovered and used as input for a subsequent round of screening for further enrichment for neutralizing clones.

We performed high-throughput sequencing analysis on each sorted library of GFP− and GFP+ cells to obtain heavy chain sequence information. We compared the frequency of heavy chain antibody variants in each population to determine the effect of the droplet neutralization assay on neutralizing and non-neutralizing antibodies in the population (FIG. 30, Table 4).

TABLE 4
Raw sequence data and fold-change enrichment calculations for SARS-
CoV-2 D614G neutralization assays. These data demonstrate the successful implementation of
droplet neutralization assays for antibodies that neutralize SARS-CoV-2, with NGS being used to
analyze the assay performance for many thousands of cells secreting polypeptide molecules in
parallel.

| | | | | Fraction | | | Fold |
|---|---|---|---|---|---|---|---|
| SARS-CoV2 S011 Round 1 | | | | of total | | | change |
| # reads | | | | reads | | | GFP−/ |
| Round 1 | Presort | GFP− | GFP+ | Presort | GFP− | GFP+ | GFP+ |
| VRC01 | 453,933 | 796,018 | 152,018 | 0.99474 | 0.9965 | 0.99153 | 1.005 |
| CR3022 | 204 | 106 | — | 0.00045 | 0.00013 | 0 | DIV/0! |
| ″910-30″ | 1,746 | 946 | 1,298 | 0.00383 | 0.00118 | 0.00847 | 0.140 |
| ″1-20″ | 452 | 1,742 | — | 0.00099 | 0.00218 | 0 | #DIV/0! |
| Total | 456,335 | 798,812 | 153,316 | I | 1 | 1 | |
| SARS-CoV2 Sort | | | | Fraction | | | Fold |
| Round 2 | | | | of total | | | change |
| # reads | | | | reads | | | GFP−/ |
| Round 2 | Presort | GFP− | GFP+ | Presort | GFP− | GFP+ | GFP+ |
| VRC01 | 1,843,790 | 1,345,524 | 985,847 | 0.97891 | 0.95775 | 0.99399 | 0.964 |
| CR3022 | 3,759 | 2,259 | 2 | 0.002 | 0.00161 | 2E−06 | 797.399 |
| ″910-30″ | 20,351 | 38,624 | 5,955 | 0.0108 | 0.02749 | 0.006 | 4.579 |
| ″1-20″ | 15,604 | 18,474 | 7 | 0.00828 | 0.01315 | 7.1E−06 | 1863.169 |
| Total | 1,883,504 | 1,404,881 | 991,811 | 1 | 1 | 1 | |

### Example 28 Application of the Single-Cell Assay Using a Synthetic Library of Antibodies with Known Neutralization Properties Against HIV Pseudoviruses Inside Emulsion Droplet Systems

This working Example relates to the successful screening of a synthetic cell library secreting antibody molecules for the neutralization of HIV pseudovirus. First, we mixed TZM-GFP cells expressing different monoclonal antibodies to generate a synthetic library consisting of 3 antibody-producing cells (the previously reported antibodies 72A1, VRC01, and VRC34); 72A1 does not neutralize HIV-1 and serves as a negative control. We used a microfluidic device to encapsulate the synthetic library with media to form droplets, with one cell per droplet. We incubated the droplet for 24 hours, allowing the secretion of IgG within the droplet for antibody accumulation. Subsequently, a second droplet containing HIV-1 BG505.W6M.Env.C2 pseudovirus was merged into the droplet with IgG expressing TZM-GFP cells. We used electrocoalescence to merge droplets, although alternative methods to merge droplets have been reported including the use of micropillar resistance arrays. The merged droplets were further incubated for another 24 hours to allow pseudovirus infection or neutralization to occur. The droplets were then broken, and the cells are recovered. Cells were allowed to recover for 48 hours. We isolated GFP- and GFP+ populations using fluorescence activated cell sorting (FACS) and extracted the gDNA from cell aliquots and performing PCR to recover the antibody gene libraries for NGS analysis. GFP− cells were also recovered and used as input for a subsequent round of screening for further enrichment for neutralizing clones.

We performed high-throughput sequencing analysis on each sorted library of GFP- and GFP+ cells to obtain heavy chain sequence information. We compared the frequency of heavy chain antibody variants in each population to determine the effect of the droplet neutralization assay on neutralizing and non-neutralizing antibodies in the population (FIG. 31, Table 5).

TABLE 5
Raw sequence data and fold-change enrichment calculations for HIV-1
W6M.Env.C2 pseudovirus neutralization assays. These data demonstrate the successful
implementation of droplet neutralization assays for antibodies that neutralize HIV-1,
with NGS being used to analyze the assay performance for many thousands
of cells secreting polypeptide molecules in parallel.
HIV-1 Sort Ro and 1

| # | | | | Fraction | | | Fold |
|---|---|---|---|---|---|---|---|
| reads | | | | of total | | | change |
| Round | | | | reads | | | GFP−/ |
| 1 | Presort | GFP− | GFP+ | Presort | GFP− | GFP+ | GFP+ |
| 72A1 | 408,194 | 17,476 | 58,341 | 0.07969 | 0.02121 | 0.1336 | 0.159 |
| VRC01 | 4,203,248 | 721,806 | 353,177 | 0.82062 | 0.87617 | 0.80875 | 1.083 |
| VRC34 | 510,606 | 84,538 | 25,175 | 0.09969 | 0.10262 | 0.05765 | 1.780 |
| Total | 5,122,048 | 823,820 | 436,693 | | | | |

### REFERENCES CITED

All references provided in this disclosure, including those listed below, are incorporated herein by reference in their entireties.
1. Mortensen, Richard M., and Robert E. Kingston. “Selection of Transfected Mammalian Cells.” Current Protocols in Molecular Biology, vol. 86, no. 1, April 2009, 10.1002/0471142727.mb0905s86.
2. DeKosky, B. J., Kojima, T., Rodin, A., Charab, W., Ippolito, G. C., Ellington, A. D. &

Georgiou, G. In-depth determination and analysis of the human paired heavy- and light-chain antibody repertoire. Nat. Med. 21, 86-91 (2015).
3. Wang, B., DeKosky, B. J., Timm, M. R., Lee, J., Normandin, E., Misasi, J., Kong, R., McDaniel, J. R., Delidakis, G., Leigh, K. E., Niezold, T., Choi, C. W., Viox, E. G., Fahad, A., Cagigi, A., Ploquin, A., Leung, K., Yang, E. S., Kong, W.-P., Voss, W. N., Schmidt, A. G., Moody, M. A., Ambrozak, D. R., Henry, A. R., Laboune, F., Ledgerwood, J. E., Graham, B. S., Connors, M., Douek, D. C., Sullivan, N. J., Ellington, A. D., Mascola, J. R. & Georgiou, G. Functional interrogation and mining of natively paired human VH: VL antibody repertoires. Nature Biotechnology 36, 152-155 (2018).
4. DeKosky, B. J., Ippolito, G. C., Deschner, R. P., Lavinder, J. J., Wine, Y., Rawlings, B. M., Varadarajan, N., Giesecke, C., Dorner, T., Andrews, S. F., Wilson, P. C., Hunicke-Smith, S. P., Willson, C. G., Ellington, A. D. & Georgiou, G. High-throughput sequencing of the paired human immunoglobulin heavy and light chain repertoire. Nat Biotech 31, 166-169 (2013).
5. As shown within the Berkeley Lights website at www.berkeleylights.com.
6. Lagerman, C. E., Lopez Acevedo, S. N., Fahad, A. S., Hailemariam, A. T., Madan, B. & DeKosky, B. J. Ultrasonically-guided flow focusing generates precise emulsion droplets for high-throughput single cell analyses. Journal of Bioscience and Bioengineering 128, 226-233 (2019).
7 Georgina To'a Salazar, et al., Antibody Therapies Targeting Complex Membrane Proteins, Engineering, Volume 7, Issue 11, 021, Pages 1541-1551.
8. Hoffmann, M., Kleine-Weber, H., Schroeder, S., Kruger, N., Herrler, T., Erichsen, S.,

Schiergens, T. S., Herrler, G., Wu, N.-H., Nitsche, A., Willer, M. A., Drosten, C. & Pohlmann, S. SARS-CoV-2 Cell Entry Depends on ACE2 and TMPRSS2 and Is Blocked by a Clinically Proven Protease Inhibitor. Cell 181, 271-280.e8 (2020).
9. Nat Protoc. 2016 March; 11(3):429-42. doi: 10.1038/nprot.2016.024. Epub 2016 Feb. 4.
10. Kulmala A, Huovinen T, Lamminmäki U. Improvement of Fab expression by screening combinatorial synonymous signal sequence libraries. Microb Cell Fact. 2019 Sep. 16; 18(1):157. doi: 10.1186/s12934-019-1210-1. PMID: 31526395; PMCID: PMC6745802.
11. Zalucki Y M, Beacham I R, Jennings M P. Biased codon usage in signal peptides: a role in protein export. Trends Microbiol. 2009 April; 17(4):146-50. doi: 10.1016/j.tim.2009.01.005. Epub 2009 Mar. 21. PMID: 19307122.
12. Zhang J, Collins A, Chen M, Knyazev 1, Mentz R. High-density perfusion culture of insect cells with a biosep ultrasonic filter. Biotechnol Bioeng. 1998 Aug. 5; 59(3):351-9. doi: 10.1002/(sici)1097-0290(19980805)59:3<351::aid-bit11>3.0.co;2-h. PMID: 10099346.
13. Arnold C E, Parekh R N, Yang W, Wittrup K D. Leader peptide efficiency correlates with signal recognition particle dependence in Saccharomyces cerevisiae. Biotechnol Bioeng. 1998 Aug. 5; 59(3):286-93. PMID: 10099339.
14. Khorshidi S, Zomorodipour A, Behmanesh M, Vatandoost J, Bos M H. Functional expression of the human coagulation factor IX using heterologous signal peptide and propeptide sequences in mammalian cell line. Biotechnol Lett. 2015 September; 37(9):1773-81. doi: 10.1007/s10529-015-1868-3. Epub 2015 Jun. 24. Erratum in: Biotechnol Lett. 2015 October; 37(10):2097. PMID: 26105559.
15. Mukhamedova M, Wrapp D, Shen C H, Gilman M S A, Ruckwardt T J, Schramm C A, Ault L, Chang L, Derrien-Colemyn A, Lucas SAM, Ransier A, Darko S, Phung E, Wang L, Zhang Y, Rush S A, Madan B, Stewart-Jones GBL, Costner P J, Holman L A, Hickman S P, Berkowitz N M, Doria-Rose N A, Morabito K M, DeKosky B J, Gaudinski M R, Chen G L, Crank M C, Misasi J, Sullivan N J, Douek D C, Kwong P D, Graham B S, P, McLellan J S, Mascola J R. Vaccination with prefusion-stabilized respiratory syncytial virus fusion protein induces genetically and antigenically diverse antibody responses. Immunity. 2021 Apr. 13; 54(4):769-780.e6. doi: 11.0.1016/j.immuni.2021.03.004. Epub 2021 Apr. 5. PMID: 33823129; PMCID: PMC8099422.
16. Fahad, A. S. et al. Functional Profiling of Antibody Immune Repertoires in Convalescent Zika Virus Disease Patients. Front. Immunol. 12, (2021).
17. Banach, B. B. et al. Paired heavy and light chain signatures contribute to potent SARS-CoV-2 neutralization in public antibody responses. bioRxiv 2020.12.31.424987 (2021) doi:10.1101/2020.12.31.424987.
18. DeKosky, B. J. et al. In-depth determination and analysis of the human paired heavy- and light-chain antibody repertoire. Nat. Med. 21, 86-91 (2015).
19. Wang, B. et al. Functional interrogation and mining of natively paired human VH:VL antibody repertoires. Nature Biotechnology 36, 152-155 (2018).
20. Tian, M. et al. Induction of HIV Neutralizing Antibody Lineages in Mice with Diverse Precursor Repertoires. Cell 166, 1471-1484.e18 (2016).
21. Jin, W., Madan, B., Mussman, B. K., Hailemariam, A. T., Fahad, A. S., Wolfe, J. R., Kwon, Y. D., Zhang, B., Shapiro, L., Kwong, P. D. & DeKosky, B. J. The Covalent SNAP Tag for Protein Display Quantification and Low-pH Protein Engineering. Journal of Biotechnology (2020). doi:10.1016/j.jbiotec.2020.06.011
22. Georgiou, G., Ippolito, G. C., Beausang, J., Busse, C. E., Wardemann, H. & Quake, S. R.

The promise and challenge of high-throughput sequencing of the antibody repertoire. Nature Biotechnology 32, 158-168 (2014).
23. Gludish D W, Boliar S, Caldwell S, Tembo D L, Chimbayo E T, Jumbo K C, Mwandumba H C, Russell D G. TZM-gfp cells: a tractable fluorescent tool for analysis of rare and early HIV-1 infection. Sci Rep. 2020 Nov. 16; 10(1):19900. doi: 10.1038/A1598-020-76422-6. PMID: 33199722; PMCID: PMC7670428,
24. Abate A R, Hung T, Mary P, Agresti J J, Weitz D A. High-throughput injection with microfluidics using picoinjectors. Proc Natl Acad Sci USA. 2010 Nov. 9; 107(45):19163-6. doi: 10.1073/pnas.1006888107. Epub 2010 Oct. 20. PMID: 20962271; PMCID: PMC2984161.
25. Seah, Y. F. S., Hu, H. & Merten, C. A. Microfluidic single-cell technology in immunology and antibody screening. Molecular Aspects of Medicine 59, 47-61 (2018).
26. Debs, B. E., Utharala, R., Balyasnikova, I. V., Griffiths, A. D. & Merten, C. A. Functional single-cell hybridoma screening using droplet-based microfluidics. Proceedings of the National Academy of Sciences 109, 11570-11575 (2012).

It will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. Any cited references are incorporated by reference herein in their entireties. In the event that there is an inconsistency between a definition of a term in the specification as compared to a definition of the term in a cited reference, the term should be interpreted based on the definition in the specification.

We claim:

1. A screening method comprising:
(a) detecting the presence and/or level of expression of a reporter molecule in a single, isolated, genetically engineered cell,
wherein the cell presents a cell surface protein; and
wherein the cell is engineered to:
(i) secrete a heterologous test polypeptide; and
(ii) express a reporter molecule if the test polypeptide activates the cell surface protein, or express a reporter molecule if the test polypeptide does not activate the cell surface protein.

2. The method of claim 1, wherein the cell comprises a mammalian cell, an insect cell, an avian cell, a yeast cell, a fungal cell, a plant cell, or a bacterial cell.

3. The method of claim 1, wherein the cell surface protein comprises an endogenous receptor.

4. The method of claim 1, wherein the cell is engineered to express the cell surface protein.

5. The method of claim 4, wherein the cell surface protein comprises a heterologous receptor.

6. The method of claim 1, wherein secretion of the test polypeptide is constitutive.

7. The method of claim 1, wherein secretion of the test polypeptide is inducible.

8. The method of claim 1, wherein the single, isolated, genetically engineered cell is in a well of a multi-well plate, a chamber of a microchip, a microfluid droplet, such as an emulsion droplet, a Nanopen™.

9. The method of claim 1, wherein the reporter molecule comprises a fluorescent marker, an enzyme, a tagged protein, or a nucleic acid sequence.

10. The method of claim 1, wherein the method further comprises sequencing the nucleic acids encoding the heterologous test polypeptide.

11. The method of claim 1, wherein heterologous test peptide comprises a variant of the cell surface receptor's ligand.

12. The method of claim 1, wherein the test polypeptide comprises an antibody or antibody-derived format, a nanobody, VHH, or antigen binding fragment thereof.

13. A composition, kit, or system, comprising the genetically engineered cell of claim 1.

14. A screening method comprising:
(a) contacting a single, isolated, genetically engineered cell with a test reagent,
wherein the cell presents a cell surface protein, and
wherein the cell is engineered to:
(i) secrete a heterologous test polypeptide; and
(ii) express a reporter molecule if one of the test polypeptide or the test reagent activates the cell surface protein;
(b) detecting the presence and/or level of expression of the reporter molecule.

15. The method of claim 14, wherein the test polypeptide comprises a variant of a cell surface protein ligand, and wherein the test reagent comprises an agonist or an antagonist of receptor activation by the wild-type ligand.

16. The method of claim 14, wherein the test reagent comprises a cell surface protein ligand, and wherein the test polypeptide is derived from a library of potential agonists or antagonists of receptor activation by the ligand.

17. A screening method comprising:
(a) contacting a single, isolated, genetically engineered cell with a test reagent comprising a reporter molecule,
wherein the cell presents a cell surface protein;
wherein the test reagent is capable of binding the cell surface protein presented by the cell, forming a reagent-protein complex, and wherein the test reagent gains entry into the cell when the reagent-receptor complex is formed;
wherein the cell is engineered to:
(i) secrete a heterologous test polypeptide;
(b) detecting the presence and/or level of expression of the reporter molecule in the cells.

18. The method of claim 17, wherein the test reagent comprises one or more of a virus, virus-like particle, pseudoviruses, and recombinant viral particle, and wherein the cell surface protein comprises a component of viral entry into the cell.

19. The method of claim 18, wherein the virus is selected from Coronavirus A, B, C, or D, Flavivirus, Herpesvirus, Lentivirus, Influenza A, B, or C.

20. The method of claim 18, wherein the virus comprises a SARS-CoV-2 virus, and wherein the cell surface protein comprises a human angiotensin-converting enzyme 2 (hACE2).
